(19) United States
(12) Patent Application Publication
(10) Pub. No.: US 2024/0100082 A1
(43) Pub. Date: Mar. 28, 2024

(54) METHODS AND COMPOSITION FOR TREATING METABOLIC SYNDROME

(71) Applicant: January, Inc., Menlo Park, CA (US)

(72) Inventors: Riya THAKKAR, Sunnyvale, CA (US); Carlo ARMIJO, San Jose, CA (US); Maziyar SABERI, San Diego, CA (US)

(21) Appl. No.: 18/201,712

(22) Filed: May 24, 2023

Related U.S. Application Data
(63) Continuation of application No. PCT/US2021/061022, filed on Nov. 29, 2021.
(60) Provisional application No. 63/119,494, filed on Nov. 30, 2020.

(51) Int. Cl. A61K 31/718; A61K 31/736; A61K 35/742; A61K 35/747; A61K 36/77; A61K 36/82; A61K 36/9066; A61P 1/00

(52) U.S. Cl. CPC A61K 31/718; A61K 31/736; A61K 35/742; A61K 35/747; A61K 36/77; A61K 36/82; A61K 36/9066; A61P 1/00

(57) ABSTRACT

The present disclosure provides methods and compositions that promote gut health. In some cases, the compositions comprise dietary fibers.

### CROSS REFERENCE

This application is a continuation of International Application No. PCT/US2021/061022, filed Nov. 29, 2021, which claims the benefit of U.S. Provisional Application No. 63/119,494, filed Nov. 30, 2020, each of which is incorporated by reference herein in its entirety.

### BACKGROUND OF THE INVENTION

One of the most common non-communicable diseases worldwide, Type 2 diabetes is a complex disease resulting from both impaired insulin secretion from the pancreas and a failure in the peripheral tissues to respond to normal insulin levels. This inability to adequately manage blood glucose levels is associated with the progressive damage, and ultimate failure, of vital organs, including the eyes, kidneys, nerves, and cardiovascular system. Currently, 463 million individuals worldwide have been diagnosed with Type 2 diabetes, and this population is expected to reach 700 million by 2045. The disease already accounts for as much as 10% of the overall healthcare budget in many countries, and in the United States alone, the total estimated 2017 cost of diagnosed diabetes is $327 billion. This includes $237 billion in direct medical costs and $90 billion in reduced productivity. There is a need for treatments which can prevent or treat Type 2 diabetes.

### SUMMARY OF THE INVENTION

In some embodiments, the present disclosure provides a dietary fiber composition comprising at least one polysaccharide selected from the group consisting of potato starch, locust bean gum, oat bran, Galacto-oligosaccharides, apple fiber, orange fiber, barley bran, oat fiber, pea fiber, chia fiber, kudzu, tara gum, konjac gum, beta glucan, guar gum, partially hydrolyzed guar gum, gum Arabic, and soluble corn fiber; and at least one component selected from the group consisting of Lactobacillus paracasei, Lactobacillus rhamnosus, Bacillus coagulans, Saccharomyces boulardii, yellow kiwi fruit extract, turmeric extract, green kiwi fruit extract and lychee fruit extract.

In some embodiments, the present disclosure provides a dietary fiber composition comprising at least two polysaccharides selected from the group consisting of potato starch, locust bean gum, oat bran, Galacto-oligosaccharides, apple fiber, orange fiber, barley bran, oat fiber, pea fiber, chia fiber, kudzu, tara gum, konjac gum, beta glucan, guar gum, partially hydrolyzed guar gum, gum Arabic, and soluble corn fiber.

In some cases, the dietary fiber composition comprises one or more of Lactobacillus paracasei, Lactobacillus rhamnosus, Bacillus coagulans, Saccharomyces boulardii, yellow kiwi fruit extract, turmeric extract, green kiwi fruit extract and lychee fruit extract. In some cases, the dietary fiber composition comprises potato starch, and locust bean gum. In some cases, the dietary fiber composition comprises an amount of potato starch from about 5% to 95%, from about 5% to about 90%, from about 5% to about 80%, from about 10% to about 50%, from about 10% to about 40%, from about 10% to about 30%, or from about 20% to about 25% by weight.

In some cases, the dietary fiber composition comprises an amount of potato starch of about 5%, 6%, 7%, 8%, 9%, 10%, 1%, 2%, %13%, %14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight. In some cases, the dietary fiber composition comprises an amount of potato starch of at least about 5%, 6%, 7%, 8%, 9%, 10%, 11%, 12%, 13%, 14%1, 5%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight.

In some cases, the dietary fiber composition comprises an amount of locust bean gum from about 5% to 95%, from about 5% to about 90%, from about 5% to about 80%, from about 10% to about 50%, from about 10% to about 30%, or from about 15% to about 20% by weight. In some cases, the dietary fiber composition comprises an amount of locust bean gum at least about 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight. In some cases, the dietary fiber composition comprises an amount of locust bean gum of about 5%, 6%, 7%, 8%, 9%, 10%, 11%, 12%, 13%1, 14%, 5%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight.

In some cases, the dietary fiber composition comprises from about 15% to about 35% potato starch and from about 10% to about 30% locust bean gum by weight.

In some cases, the dietary fiber composition comprises oat bran. In some cases, the dietary fiber composition comprises an amount of oat bran from about 5% to 95%, from about 5% to about 90%, from about 5% to about 80%, from about 10% to about 50%, from about 10% to about 40%, from about 20% to about 40%, or from about 25% to about 35% by weight. In some cases, the dietary fiber composition comprises an amount of oat bran of at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight. In some cases, the dietary fiber composition comprises an amount of oat bran of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight.

In some cases, the dietary fiber composition comprises one or more of Galacto-oligosaccharides, barley bran, apple fiber, orange fiber, oat fiber, pea fiber, kudzu or chia.

In some cases, the dietary fiber composition comprises an amount of Galacto-oligosaccharides, barley bran, apple fiber, orange fiber, oat fiber, pea fiber, kudzu or chia from about 5% to 95%, from about 5% to about 90%, from about 5% to about 80%, from about 10% to about 50%, from about 10% to about 40%, from about 15% to about 25%, or from about 5% to about 15% by weight. In some cases, the dietary fiber composition comprises an amount of Galacto-oligosaccharides of at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight. In some cases, the dietary fiber composition comprises an amount of Galacto-oligosaccharides of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight.

In some cases, the dietary fiber composition comprises Galacto-oligosaccharides and apple fiber. In some cases, the dietary fiber composition comprises about 23% potato starch, about 19% locust bean gum, about 27.5% oat bran, about 20% Galacto-oligosaccharides and about 10.5% apple fiber by weight. In some cases, the dietary fiber composition comprises 23% resistant potato starch, 19% locust bean gum, 27.5% oat bran, 10.5% Galacto-oligosaccharides and 20% apple fiber by weight.

In some cases, the dietary fiber composition comprises orange fiber and barley bran. In some cases, the dietary fiber composition comprises 23% potato starch, 19% locust bean gum, 27.5% oat bran, 20% orange fiber and 10.5% barley bran by weight.

In some cases, the dietary fiber composition comprises apple fiber and oat fiber. In some cases, the dietary fiber composition comprises 23% potato starch, 19% locust bean gum, 27.5% oat bran, 20% apple fiber and 10.5% oat fiber by weight.

In some cases, the dietary fiber composition comprises barley bran and pea fiber. In some cases, the dietary fiber composition comprises 23% potato starch, 19% locust bean gum, 27.5% oat bran, 20% barley bran and 10.5% pea fiber by weight

In some cases, the dietary fiber composition comprises kudzu and chia. In some cases, the dietary fiber composition comprises 23% potato starch, 19% locust bean gum, 27.5% oat bran, 20% chia and 10.5% kudzu by weight

In some cases, the dietary fiber composition comprises beta glucan. In some cases, the dietary fiber composition comprises an amount of beta glucan from about 5% to 95%, from about 5% to about 90%, from about 5% to about 80%, from about 10% to about 50%, from about 10% to about 40%, from about 20% to about 40%, or from about 25% to about 35% by weight. In some cases, the dietary fiber composition comprises an amount of beta glucan of at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight. In some cases, the dietary fiber composition comprises an amount of beta glucan of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight.

In some cases, the dietary fiber composition comprises one or more of Galacto-oligosaccharides, partially hydrolyzed guar gum, pea fiber, gum arabic, soluble corn fiber, chia fiber, or kudzu starch. In some cases, the dietary fiber composition comprises an amount of Galacto-oligosaccharides, partially hydrolyzed guar gum, pea fiber, gum arabic, soluble corn fiber, chia fiber, or kudzu starch from about 5% to 95%, from about 5% to about 90%, from about 5% to about 80%, from about 10% to about 50%, from about 10% to about 40%, or from about 5% to about 25% by weight. In some cases, the dietary fiber composition comprises an amount of Galacto-oligosaccharides, partially hydrolyzed guar gum, pea fiber, gum arabic, soluble corn fiber, chia fiber, or kudzu starch of at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight. In some cases, the dietary fiber composition comprises an amount of Galacto-oligosaccharides, partially hydrolyzed guar gum, pea fiber, gum Arabic, soluble corn fiber, chia fiber, or kudzu starch of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% by weight.

In some cases, the dietary fiber composition comprises Galacto-oligosaccharides and partially hydrolyzed guar gum. In some cases, the dietary fiber composition comprises 23% potato starch, 19% locust bean gum, 27.5% concentrated barley beta glucan, 23% Galacto-oligosaccharides and 7.5% partially hydrolyzed guar gum. In some cases, the dietary fiber composition comprises 23% potato starch, 19% locust bean gum, 27.5% concentrated barley beta glucan, 15.5% Galacto-oligosaccharides and 15% partially hydrolyzed guar gum.

In some cases, the dietary fiber composition comprises pea fiber and partially hydrolysed guar gum. In some cases, the dietary fiber composition comprises 23% potato starch, 19% locust bean gum, 27.5% concentrated barley beta glucan, 20% Galacto-oligosaccharides and 10.5% pea fiber.

In some cases, the dietary fiber composition comprises pea fiber and gum arabic. In some cases, the dietary fiber composition comprises 23% potato starch, 19% locust bean gum, 27.5% concentrated barley beta glucan, 20% gum Arabic and 10.5% pea fiber.

In some cases, the dietary fiber composition comprises soluble corn fiber and partially hydrolysed guar gum. In some cases, the dietary fiber composition comprises 23% potato starch, 19% locust bean gum, 27.5% concentrated barley beta glucan, 20% soluble corn fiber and 10.5% partially hydrolysed guar gum.

In some cases, the dietary fiber composition comprises chia and kudzu. In some cases, the dietary fiber composition comprises 23% potato starch, 19% locust bean gum, 27.5% concentrated barley beta glucan, 20% chia fiber and 10.5% kudzu starch.

In some embodiments, the present disclosure provides a method of promoting health in a subject, the method comprising administering to the subject an effective amount of a dietary fiber composition as described herein. In some cases, promoting health comprises promoting gut health, treating metabolic syndrome, glucose regulation, satiety, immune function or lipid control. In some cases, promoting health comprises increasing production of at least one short-chain fatty acid in the digestive tract of the subject. In some cases, the short-chain fatty acid is acetate, propionate, or butyrate. In some cases, promoting health comprises increasing abundance of one or more keystone microbial species in the digestive tract of the subject.

In some cases, the keystone microbial species is Faecalibacterium prausnitzii, Akkermansia muciniphila, Collinsella aerofaciens, Eubacterium hallii, Bacteroides thetaiotaomicron, Roseburia hominis, or Eubacterium rectale. In some cases, promoting health comprises decreasing abundance of one or more pathogenic microbial species in the digestive tract of the subject. In some cases, the pathogenic microbial species is Clostridioides difficile, Shigella sonnei, Escherichia coli, Campylobacter, Shigella flexneri, Shigella dysenteriae, Shigella boydii, Campylobacter gracilis, Citrobacter freundii, or Citrobacter braakii. In some cases, promoting health comprises treating antibiotic caused dysbiosis. In some cases, promoting health comprises increasing a Bacteroidetes to Firmicutes ratio in the digestive tract of the subject.

In some cases, promoting health comprises increasing secretion of GLP-1, PYY, or IL-10 in the digestive tract of the subject. In some cases, promoting health comprises increasing expression of Gcg, Pcsk1, Pyy, Tlr4, Muc1, or Muc2 in the digestive tract of the subject. In some cases, the subject has type 2 diabetes.

In some embodiments, the present disclosure provides a composition for maintaining health in a healthy subject, the composition comprising of potato starch, locust bean gum, concentrated β-glucan, Galacto-oligosaccharides and partially hydrolyzed guar gum. In some embodiments, the present disclosure provides a composition for maintaining glucose regulation in a healthy subject, the composition comprising of resistant potato starch, locust bean gum, concentrated β-glucan, partially hydrolyzed guar gum, and pea fiber. In some embodiments, the present disclosure provides a composition for promoting immune function in a healthy subject, the composition comprising of potato starch, locust bean gum, concentrated β-glucan, soluble corn fiber, and partially hydrolyzed guar gum. In some embodiments, the present disclosure provides a composition for promoting lipid regulation in a healthy subject, the composition comprising of potato starch, locust bean gum, concentrated β-glucan, chia and kudzu. In some embodiments, the present disclosure provides a composition comprising at least two of yellow kiwi fruit extract, turmeric extract, green kiwi fruit extract, lychee fruit extract, Lactobacillus paracasei, Lactobacillus rhamnosus, Bacillus coagulans and Saccharomyces boulardii.

In some cases, the composition comprises about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% yellow kiwi fruit extract by weight. In some cases, the composition comprises about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% turmeric extract by weight. In some cases, the composition comprises about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% green kiwi fruit extract by weight. In some cases, the composition comprises about 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% lychee fruit extract by weight.

In some cases, the composition comprises about 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35% 36%, 37%, 38%, 39%, 40%, 45% or 50% Lactobacillus paracasei by weight. In some cases, the composition comprises about 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% Lactobacillus rhamnosus by weight. In some cases, the composition comprises about 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% Bacillus coagulans by weight. In some cases, the composition comprises about 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% Saccharomyces boulardii by weight.

In some cases, the composition comprises about 40% yellow kiwi fruit extract, 17% turmeric extract, 40% green kiwi fruit extract, and 3% lychee fruit extract by weight.

In some cases, the composition promotes health in a healthy individual. In some cases, the dietary fiber composition promotes health in a subject with type 2 diabetes.

In some cases, the composition comprises Lactobacillus paracasei, Lactobacillus rhamnosus, Bacillus coagulans and Saccharomyces boulardii.

In some cases, the dietary fiber composition is provided as a tablet, capsule, powder blend, gummy, nutritional bars, liquid formulations.

In some embodiments, the present disclosure provides a method for promoting health in a subject, the method comprising administering a composition as described herein. In some cases, promoting health comprises improving or maintaining health. In some cases, promoting health comprises improving or maintaining gut health, glucose regulation, satiety, immune function or lipid control. In some cases, promoting health comprises improving regularity or consistency of bowel movements. In some cases, the subject is a healthy subject. In some cases, the subject has pre-diabetes. In some cases, the subject has type 2 diabetes. In some cases, the subject has inflammatory bowel disease or inflammatory bowel syndrome.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates cholesterol content relative to a blank sample. Error bars are coefficient of variations (CVs) between replicates of the screen (N=3). Blank: yolk emulsion without fiber. SYN001: Positive control, Fructooligosaccharide (Sigma Aldrich) with yolk emulsion. Statistical significance against baseline was measured using 2-way ANOVA Tukey's multiple comparison test, ****=P-value<0.0001, ***=P-value<0.001, **=P-value<0.01, *=P-value<0.05.

FIG. 2A illustrates average gas production for 3 Healthy Donors. Error bars represent CV (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents gas production in mM during the process of fermentation. Out of the 24 hr and 48 hr time points, the higher gas production value was used for plotting this graph. Statistical significance against SYN001 was measured using 2-way ANOVA Tukey's multiple comparison test, ****=P-value<0.0001, ***=P-value<0.001.

FIG. 2B illustrates average gas production for 3 Type 2 Diabetes Donors. Error bars represent CV in gas production (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents gas production in mM during the process of fermentation. Out of the 24 hr and 48 hr time points, the higher gas production value was used for plotting this graph. Statistical significance against SYN001 was measured using 2-way ANOVA Tukey's multiple comparison test, ****=P-value<0.0001, ***=P-value<0.001.

FIG. 3A illustrates pH reduction for 3 Healthy Donors. Error bars represent CV in pH reduction (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the reduction in pH from baseline (baseline pH=7) during the process of fermentation. Out of the 24 hr and 48 hr time point, the higher pH reduction value was used for plotting this graph. Statistical significance against SYN001 was measured using 2-way ANOVA Tukey's multiple comparison test, ****=P-value<0.0001, ***=P-value<0.001.

FIG. 3B illustrates pH reduction for 3 Type 2 Diabetes Donors. Error bars represent CV in pH reduction (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the reduction in pH from baseline (baseline pH=7) during the process of fermentation. Out of the 24 hr and 48 hr time point, the higher pH reduction value was used for plotting this graph. Statistical significance against SYN001 was measured using 2-way ANOVA Tukey's multiple comparison test, ****=P-value<0.0001, ***=P-value<0.001.

FIG. 4A illustrates acetate production post in vitro fermentation for 3 healthy donors using GC-FID. Error bars represent CV in acetate production (N=3). Blank—No carbon substrate, SYN001-Fast fermenting positive control (Fructooligosaccharide-Sigma Aldrich), JAN001-JAN031—Prebiotic fiber candidates as listed in Table 1. Y-axis represents the amount of acetate produced in mM during the process of stool and fiber fermentation. Out of 24 h and 48 h, the higher acetate value was used for plotting this graph. Statistical significance against SYN001 was measured using 2-way ANOVA Tukey's multiple comparison test, ****=P-value<0.0001, ***=P-value<0.001, **=P-value<0.01.

FIG. 4B illustrates acetate production post in vitro fermentation for 3 Type 2 Diabetes donors using GC-FID. Error bars represent CV in acetate production (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the amount of acetate produced in mM during the process of stool and fiber fermentation. Out of 24 h and 48 h, the higher acetate value was used for plotting this graph. Statistical significance against SYN001 was measured using 2-way ANOVA Tukey's multiple comparison test, ****=P-value<0.0001, ***=P-value<0.001, **=P-value<0.01.

FIG. 4C illustrates propionate production post in vitro fermentation for 3 healthy donors using GC-FID. Error bars represent CV in propionate production (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the amount of propionate produced in mM during the process of stool and fiber fermentation. Out of 24 h and 48 h, the higher propionate value was used for plotting this graph. Statistical significance against baseline was measured using 2-way ANOVA Tukey's multiple comparison test, ****=P-value<0.0001, ***=P-value<0.001, **=P-value<0.01.

FIG. 4D illustrates propionate production post in vitro fermentation for 3 Type 2 Diabetes donors using GC-FID. Error bars represent CV in propionate production (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the amount of propionate produced in mM during the process of stool and fiber fermentation. Out of 24 h and 48 h, the higher propionate value was used for plotting this graph. Statistical significance against baseline was measured using 2-way ANOVA Tukey's multiple comparison test, ****=P-value<0.0001, ***=P-value<0.001, **=P-value<0.01, *=P-value<0.1.

FIG. 4E illustrates butyrate production post in vitro fermentation for 3 healthy donors using GC-FID. Error bars represent CV in butyrate production (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the amount of butyrate produced in mM during the process of stool and fiber fermentation. Out of 24 h and 48 h, the higher butyrate value was used for plotting this graph. Statistical significance against baseline was measured using Tukey's multiple comparison test, ****=P-value<0.0001, ***=P-value<0.001, **=P-value<0.01.

FIG. 4F illustrates butyrate production post in vitro fermentation for 3 Type 2 Diabetes donors using GC-FID. Error bars represent CV in butyrate production (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the amount of butyrate produced in mM during the process of stool and fiber fermentation. Out of 24 h and 48 h, the higher butyrate value was used for plotting this graph. Statistical significance against baseline was measured using Tukey's multiple comparison test, ****=P-value<0.0001, ***=P-value<0.001, **=P-value<0.01.

FIG. 4G illustrates total short-chain fatty acids (Acetate, Propionate, and Butyrate) production post in vitro fermentation for 3 healthy donors using GC-FID. Error bars represent CV in Total short-chain fatty acids production (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the sum of acetate, propionate, and butyrate in mM during the process of stool and fiber fermentation. Acetate, propionate, and butyrate production values from FIGS. 6a, 6c, and 6e were added for Total short-chain fatty acids. Statistical significance against baseline was measured using 2-way ANOVA Tukey's multiple comparison test, ****=P-value<0.0001, ***=P-value<0.001, **=P-value<0.01.

FIG. 4H illustrates total short-chain fatty acids (Acetate, Propionate, and Butyrate) production post in vitro fermentation for 3 Type 2 diabetes donors using GC-FID. Error bars represent CV in Total short-chain fatty acids (N=3). Blank—No carbon substrate, SYN001—Fast fermenting positive control (Fructooligosaccharide-Sigma Aldrich), JAN001-JAN031—Prebiotic fiber candidates as listed in Table 1. Y-axis represents the sum of acetate, propionate, and butyrate in mM during the process of stool and fiber fermentation. Acetate, propionate, and butyrate production values from FIGS. 6b, 6d, and 6f were added for Total short-chain fatty acids. Statistical significance against baseline was measured using 2-way ANOVA Tukey's multiple comparison test, ****=P-value<0.0001, ***=P-value<0.001, **=P-value<0.01.

FIG. 5A illustrates a heatmap of potentially pathogenic gut microbial species found in each of three healthy donors. Z-score assigned by a significant difference in relative abundance from initial fecal and blank fermentation samples. Values normalized across each fiber condition per one species to evaluate the specificity of fiber condition to bacterial growth. SYN001 is again included as an inter-experimental control. Z-scores of above or below ±2.575829 has a p-value of <0.01.

FIG. 5B illustrates a heatmap of commercially approved bacterial probiotic species found in each healthy donor. Z-score assigned by a significant difference in relative abundance from initial fecal and blank fermentation samples. Values normalized across each fiber condition per one species to evaluate the specificity of fiber condition to bacterial growth. SYN001 is again included as an inter-experimental control. Z-scores of above or below ±2.575829 has a p-value of <0.01.

FIG. 5C illustrates a heatmap of gut bacterial species associated with the amelioration metabolic syndrome symptoms found in each healthy donor. Z-score assigned by a significant difference in relative abundance from initial fecal and blank fermentation samples. Values normalized across each fiber condition per one species to evaluate the specificity of fiber condition to bacterial growth. SYN001 is again included as an inter-experimental control. Z-scores of above or below ±2.575829 has a p-value of <0.01.

FIG. 5D illustrates a heatmap of potentially pathogenic gut microbial species found in each Type 2 Diabetic donor. Z-score assigned by a significant difference in relative abundance from initial fecal and blank fermentation samples. Values normalized across each fiber condition per one species to evaluate the specificity of fiber condition to bacterial growth. SYN001 is again included as an inter-experimental control. Z-scores of above or below ±2.575829 has a p-value of <0.01.

FIG. 5E illustrates a heatmap of probiotic bacterial strains found in each Type 2 Diabetic donor. Z-score assigned by a significant difference in relative abundance from initial fecal and blank fermentation samples. Values normalized across each fiber condition per one species to evaluate the specificity of fiber condition to bacterial growth. SYN001 is again included as an inter-experimental control. Z-scores of above or below ±2.575829 has a p-value of <0.01.

FIG. 5F illustrates a heatmap of gut bacterial species associated with the amelioration metabolic syndrome symptoms found in each healthy donor. Z-score assigned by a significant difference in relative abundance from initial fecal and blank fermentation samples. Values normalized across each fiber condition per one species to evaluate the specificity of fiber condition to bacterial growth. SYN001 is again included as an inter-experimental control. Z-scores of above or below ±2.575829 has a p-value of <0.01.

FIG. 6A illustrates a Bacteroidetes to Firmicutes ratio across three healthy donors. The averaged ratio of relative abundance values between both phyla as higher ratios are associated with lesser symptoms of diabetes. Error bars represent the CV between donors. Initial samples are from the initial fecal aliquot prior to fermentation. Initial samples were used instead of the blank since it is more representative of the ratio of bacteroidetes/firmicutes in each donor's colon than the blank which is skewed by the fermentation.

FIG. 6B illustrates a Bacteroidetes to Firmicutes ratio across three Type 2 Diabetic donors. The averaged ratio of relative abundance values between both phyla as higher ratios are associated with lesser symptoms of diabetes. Error bars represent the CV between donors. Initial samples are from the initial fecal aliquot prior to fermentation. Initial samples were used instead of the blank since it is more representative of the ratio of bacteroidetes/firmicutes in each donor's colon than the blank which is skewed by the fermentation. Statistical significance against baseline was measured using Tukey's multiple comparison test, ****=P-value<0.0001, **=P-value<0.01.

FIG. 7A illustrates an average relative secretion of GLP-1 across 3 Healthy Donors. Values are the ratio of each condition compared to the basal blank fecal fermentation sample. Error bars are the CV between donors. The dotted line is the level of the positive control. Conditions with 200 mM of butyrate in basal media as positive control.

FIG. 7B illustrates an average relative secretion of GLP-1 across 3 Type 2 Diabetic Donors. Values are the ratio of each condition compared to the basal blank fecal fermentation sample. Error bars are the CV between donors. The dotted line is the level of the positive control. Conditions with 200 mM of butyrate in basal media as a positive control.

FIG. 7C illustrates an average relative secretion of PYY across 3 Healthy Donors. Values are the ratio of each condition compared to the basal blank fecal fermentation sample. Error bars are the CV between donors. The dotted line is the level of the positive control. Conditions with 200 mM of butyrate in basal media as a positive control.

FIG. 7D illustrates an average relative secretion of PYY across 3 Type 2 Diabetic Donors. Values are the ratio of each condition compared to the basal blank fecal fermentation sample. Error bars are the CV between donors. The dotted line is the level of the positive control. Conditions with 200 mM of butyrate in basal media as a positive control.

FIG. 7E illustrates an average relative secretion of IL-10 across 3 Healthy Donors. Values are the ratio of each condition compared to the basal blank fecal fermentation sample. Error bars are the CV between donors. The dotted line is the level of the positive control. Conditions with 200 mM of butyrate in basal media as a positive control.

FIG. 7F illustrates an average relative secretion of IL-10 across 3 Type 2 Diabetic Donors. Values are the ratio of each condition compared to the basal blank fecal fermentation sample. Error bars are the CV between donors. The dotted line is the level of the positive control. Conditions with 200 mM of butyrate in basal media as a positive control.

FIG. 8A illustrates the relative expression of Gcg across 3 Healthy Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control. Statistical significance by Tukey's multiple comparison test. ****=P-value<0.0001, **=P-value<0.01.

FIG. 8B illustrates an average relative expression of Gcg across 3 Healthy Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 8C illustrates an average relative expression of Pcsk1 across 3 Healthy Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control. ***=P-value<0.001.

FIG. 8D illustrates an average relative expression of Pcsk1 across 3 Type 2 Diabetic Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 8E illustrates an average relative expression of Pyy across 3 Healthy Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media acted as a positive control. *=P-value<0.05.

FIG. 8F illustrates an average relative expression of Pyy across 3 Type 2 Diabetic Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 8G illustrates an average relative expression of Tlr4 across 3 Healthy Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 8H illustrates an average relative expression of Tlr4 across 3 Type 2 Diabetic Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 8I illustrates an average relative expression of Muc1 across 3 Healthy Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 8J illustrates an average relative expression of Muc1 across 3 Type 2 Diabetic Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 8K illustrates an average relative expression of Muc2 across 3 Healthy Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 8L illustrates an average relative expression of Muc2 across 3 Type 2 Diabetic Donors. Values are 2(ΔΔCt) of each condition compared to the basal blank fecal fermentation sample with the normalization reference gene: Gapdh. Error bars are the CV between donors. The dotted line is the level of the positive control. Conditions with 200 mM of butyrate in basal media as a positive control.

FIG. 9A illustrates total indole content present in January high and low efficacious prebiotic fibers for healthy Donor 1 in post-fermentation supernatants. Black bars represent high efficacy fibers and grey bars represent low efficacy fibers. Blank samples contain no carbon source. SYN001 is used as a fast fermenting positive control.

FIG. 9B illustrates total indole content present in January high and low efficacious prebiotic fibers for healthy Donor 2 in post-fermentation supernatants. Black bars represent high efficacy fibers and grey bars represent low efficacy fibers. Blank samples contain no carbon source. SYN001 is used as a fast fermenting positive control.

FIG. 9C illustrates total indole content present in January high and low efficacious prebiotic fibers for healthy Donor 3 in post-fermentation supernatants. Black bars represent high efficacy fibers and grey bars represent low efficacy fibers. Blank samples contain no carbon source. SYN001 is used as a fast fermenting positive control.

FIG. 10 illustrates an average gas production for polyphenol spiked complete blends across 3 Type 2 Diabetic Donors. The above figure shows gas production in mL post 24 h fermentation for 12 January bioactive compounds in combination with a prebiotic fiber blend. ‘Blend’ sample is 25% Resistant Potato Starch, 9% Sunfiber, 24% Galactooligosaccharides, 26% Concentrated Barley Beta Glucan, and 16% Locust Bean Gum. Blank contains no carbon source, SYN001 is fast fermenting positive control. Error bars represent the CV between three Type 2 diabetes donors.

FIG. 11 illustrates an average pH reduction for polyphenol spiked complete blends across 3 Type 2 Diabetic Donors Above figure shows pH reduction post 24 h fermentation for 12 January bioactive compounds in combination with a prebiotic fiber blend. ‘Blend’ sample is the lead prebiotic fiber blend from January's weighing system used across the experiment without any Bioactive compound. Blank contains no carbon source, SYN001 is fast fermenting positive control. Error bars represent the CV between three Type 2 diabetes donors. Blend+Bio1 and Blend+Bio4 are the two candidates.

FIG. 12 illustrates an average total short-chain fatty acids production for polyphenol spiked complete blends across 3 Type 2 Diabetic Donors. Dotted line portrays baseline of the complete blend. Error bars represent CV between Donors. Statistical significance against baseline was measured using 2-way ANOVA Tukey's multiple comparison test, ***=P-value<0.001, **=P-value<0.01, *=P-value<0.05.

FIGS. 13A and 13B illustrate gas production with healthy and T2D samples respectively.

FIGS. 14A and 14B illustrate pH with healthy and T2D samples respectively.

FIGS. 15A and 15B illustrate total SCFAs with healthy and T2D samples respectively.

FIGS. 16A and 16B illustrate GLP-1 secretion with healthy and T2D samples respectively.

FIGS. 17A and 17B illustrate PYY secretion with healthy and T2D samples respectively.

FIGS. 18A and 18B illustrate IL-10 secretion with healthy and T2D samples respectively.

FIGS. 19A and 19B illustrate Gcg expression with healthy and T2D samples respectively.

FIGS. 20A and 20B illustrate Pcsk1 expression with healthy and T2D samples respectively.

FIGS. 21A and 21B illustrate Pyy expression with healthy and T2D samples respectively.

FIGS. 22A and 22B illustrate Tlr4 expression with healthy and T2D samples respectively.

FIGS. 23A and 23B illustrate Muc expression with healthy and T2D samples respectively.

FIGS. 24A and 24B illustrate Muc2 expression with healthy and T2D samples respectively.

FIG. 25 illustrates cholesterol reduction by the different fibers.

FIGS. 26A and 26B illustrate Bacteroidetes/Firmicutes ratio with healthy and T2D samples respectively.

FIG. 27 illustrates general conditions of the disclosed experiments.

FIGS. 28A-28C illustrate the results of experiments that show that use of the product JAN1000 selectively increases production of SCFA and medium-chain fatty acids (MCFA).

FIGS. 29A-29B illustrate JAN1000's effect on chain elongation.

FIGS. 30A-30E illustrate the comparative effect of JAN1000 on regulators of metabolic health.

FIGS. 31A-31D illustrate the comparative effect JAN1000 on regulators of intestinal immune system and mucosal integrity

FIGS. 32A-32B illustrate results showing that JAN1000 not only promotes carbohydrate fermentation, but it has the ability to boost microbial metabolism of other macromolecules.

FIG. 33 shows a plot that illustrates associations of SCFA and MCFA productions with host-secreted hormones and cytokines.

FIG. 34 illustrates a plot that shows JAN1000's effects on the production of health promoting neurotransmitters and metabolites.

FIG. 35 illustrates a plot that shows JAN1000's effects on promoting growth of various types of bacteria.

FIGS. 36A-36F illustrate the capability of JAN1000 to grow existing low populations of butyrogenic species.

FIGS. 37A-37B illustrates a plot that shows JAN1000's effectiveness as a carbon source for particular bacteria when compared to inulin and Psyllium.

FIG. 38 illustrates a plot that shows JAN1000's effect on opportunistic pathogens.

FIGS. 39A-39B illustrates experimental results showing how JAN1000's effect on Bilophila wadsworthia.

FIG. 40 illustrates JAN1000's effect on detrimental microbes. JAN1000 suppresses pathogenic and detrimental microbial pathways.

FIG. 41 illustrates how diverse monosaccharides that form complex polysaccharides have great potential to recruit fermentative bacterial consortiums.

FIG. 42 illustrates comparisons of composition of JAN1000 to those of supplements Inulin and Metamucil®.

FIGS. 43A-43B illustrate that JAN1000 is rich in fermentative polysaccharides like galactomannan, β-glucan and resistant starch type 2.

FIGS. 44A-44C illustrate that JAN1000 elicits a stronger fermentation profile than inulin and Metamucil®.

FIG. 45 illustrates that a disclosed experiment compares the propensity for a donor's microbiota to produce short-chain fatty acids (SCFAs) from JAN1000, inulin, and Metamucil®.

FIGS. 46A-46C illustrate an experiment illustrating effects of JAN1000, inulin, and Metamucil® on production of butyrate and acetate in healthy patients.

FIG. 47 illustrates an experiment in which anti-cholesterol potential of JAN1000 is compared to that of inulin and Metamucil® in an in vitro model.

FIG. 48 illustrates an experiment comparing the reduction of cholesterol by JAN1000 to that of inulin and Metamucil®.

FIG. 49 illustrates an experiment to evaluate the modulatory effect of JAN1000 on intestinal microbiome.

FIGS. 50A-50B illustrate experimental results showing JAN1000's effects on populations of various microorganisms.

FIG. 51 illustrates an experiment which is a human clinical study designed to test product quality and satisfaction, as well as to get an early read on satiety, blood glucose, and improvements to gut and metabolic health.

FIG. 52 illustrates a flow chart of the experiment from FIG. 51.

FIGS. 53A-53B illustrate an experimental design for collecting samples and testing them with the supplement JAN1000.

FIGS. 54A-54C illustrate results of JAN1000 improves overall glucose homeostasis and insulin sensitivity.

FIGS. 55A-55C illustrate a comparison of Psyllium husk with JAN1000 on time-in-range.

FIGS. 56A-56E illustrate comparative effects of JAN100 on lipid homeostasis.

FIGS. 57A-57B illustrate a rice challenge experiment. In a control, glucose is monitored for a subject just after and 120 minutes after eating rice.

FIGS. 58A-58B illustrate comparative effects of JAN1000 and Psyllium husk on hyperglycemic and hypoglycemic episodes.

FIGS. 59A-59B illustrate a case study of JAN1000's effects on a healthy participant.

FIGS. 60A-60C illustrate additional implications of the case study from FIGS. 59A-59B.

FIGS. 61A-61D illustrate additional implications of the case study from FIGS. 59A-59B.

FIGS. 62A-62C illustrate an experiment to determine whether JAN1000 spikes blood glucose in healthy and participants with type 2 diabetes. During this experiment, blood samples were taken in non-fasted state, at a consistent time of day in the afternoon for 3 consecutive days.

FIGS. 63A-63C illustrate an experiment to determine if a single serving of JAN1000 modulates key hormones, peptides and cytokines involved in glucoregulation, satiety, and immune control in a healthy participant.

FIGS. 64A-64C illustrate another iteration of the rice experiment.

### DETAILED DESCRIPTION OF THE INVENTION

Individuals with type 2 diabetes and pre-diabetes may have altered intestinal microbiota profiles compared to healthy individuals. The present disclosure provides a series of synbiotic supplements may ameliorate indications associated with diabetic symptoms. Current advances in the field of fiber microbiome research suggest that not only the amount of dietary fiber but also that polysaccharide composition plays a key role in driving the metabolic effect. These specific polysaccharides derived metabolites, especially short-chain fatty acids, can then act upon the enteroendocrine L cells and enterocytes of the colon to induce beneficial host hormones.

Key hormone targets in this pipeline are GLP-1, PYY, and IL-10. GLP-1 is an incretin peptide secreted by the enteroendocrine cells of the lower gastrointestinal tract and may play a key role in a host's response in regulating blood glucose. Induction of GLP-1 can be mediated in response to increased intestinal butyrate production by the resident microbiota, linking the induction of this peptide by short-chain fatty acids. In addition to inducing physical bulking for satiety, fermented dietary fiber may also modulate PYY secretion from the enteroendocrine cells of the lower gastrointestinal tract. PYY is a satiety hormone determining and controlling hunger and fullness, which are attributes that could benefit weight loss. Outside of glucose control and managing eating habits, low-grade chronic inflammation promotes insulin resistance and pathogenesis of Type 2 diabetes among other cardiometabolic conditions. IL-10, also recognized as a master regulator of the immune system, is a genetically and clinically validated anti-inflammatory cytokine and its target modulation has been shown to improve intestinal inflammation and systemic insulin sensitivity produced in humans shown to have localized anti-inflammatory effects along the gastrointestinal tract in addition to beneficial effects on the entire host immunity. This anti-inflammatory cytokine is produced in low amounts by the enterocytes of the colon and through this pipeline, we show that microbial-derived metabolites also drive increased production.

### Dietary Fibers

In some embodiments, a composition for promoting health in a subject comprises potato starch. The potato starch may be a resistant potato starch, a non-resistant potato starch, or an unmodified potato starch. In some cases, the composition comprises an amount of potato starch from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of potato starch of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of potato starch of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of potato starch of less than about 1%, 2%, 3%, 4%, 5% 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises locust bean gum. In some cases, the composition comprises an amount of locust bean gum from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of locust bean gum of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of locust bean gum of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of locust bean gum of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises oat bran. In some cases, the composition comprises an amount of oat bran from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of oat bran of about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of oat bran of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of oat bran of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises Galacto-oligosaccharides. In some cases, the composition comprises an amount of Galacto-oligosaccharides from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of Galacto-oligosaccharides of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of Galacto-oligosaccharides of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of Galacto-oligosaccharides of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises barley bran. In some cases, the composition comprises an amount of barley bran from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of barley bran of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of barley bran of at least about 1%, 2%, 3%, 4%, 5%, 6%, 7%, 8%, 9%, 10%, 1%, 2%, %13%, %14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of barley bran of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises apple fiber. In some cases, an apple fiber may be a composition derived from apples and comprising an apple fiber and other compounds. In some cases, an apple fiber may be dried apple peal. In some cases, the composition comprises an amount of apple fiber from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of apple fiber of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of apple fiber of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of apple fiber of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises orange fiber. In some cases, the composition comprises an amount of orange fiber from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of orange fiber of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of orange fiber of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of orange fiber of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises oat fiber. In some cases, the composition comprises an amount of oat fiber from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of oat fiber of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of oat fiber of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of oat fiber of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises pea fiber. In some cases, the composition comprises an amount of pea fiber from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of pea fiber of about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of pea fiber of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of pea fiber of less than about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises kudzu. In some cases, the composition comprises an amount of kudzu from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of kudzu of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of kudzu of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of kudzu of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises chia. In some cases, the composition comprises an amount of chia from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of chia of about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of chia of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of chia of less than about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises beta glucan. The beta glucan may be a concentrated beta glucan. The beta glucan may be a barley beta glucan, an oat beta glucan, or a seaweed beta glucan. In some cases, the composition comprises an amount of beta glucan from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of beta glucan of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of beta glucan of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of beta glucan of less than about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises galacto-oligosaccharides. In some cases, the composition comprises an amount of galacto-oligosaccharides from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of galacto-oligosaccharides of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of galacto-oligosaccharides of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of galacto-oligosaccharides of less than about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises partially hydrolyzed guar gum. In some cases, the composition comprises an amount of partially hydrolyzed guar gum from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of partially hydrolyzed guar gum of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of partially hydrolyzed guar gum of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of partially hydrolyzed guar gum of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises gum arabic. In some cases, the composition comprises an amount of gum arabic from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of gum arabic of about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of gum arabic of at least about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of gum arabic of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises an inulin. An inulin may be a fructooligosaccharide, an agave inulin, a chicory root inulin, a Jerusalem artichoke inulin, or any other inulin. In some cases, the composition comprises an amount of inulin from about 5% to 95%, from about 5% to about 90%, from about 5% to about 80%, from about 10% to about 50%, from about 10% to about 40%, from about 10% to about 30%, or from about 20% to about 25% by weight.

Other dietary fibers which may be included in a composition described herein include wheat fiber, wheat dextrin, rice fiber, rice starch, gum acacia, Psyllium fiber, xanthum gum, banana starch, banana resistant starch, green banana starch, green banana resistant starch, cassava starch, tapioca starch, shitake mushroom power, corn fiber, corn bran, corn dextrin, pectin gum, low methoxy apple pectin, low methoxy citrus pectin, mixed fruit pectin, barley bran, seaweed beta glucan, tapioca fiber, Arabinoxylan oligosaccharide, wheat bran, oat bran, bulgur bran, chia seed flour, isomaltodextrin, alginate, ancient grain teff fiber, polydextrose, isomaltooligosaccharide, nutriose, sugarcane fiber, sugar beet fiber, bamboo fiber, hemp fiber, saw palmetto fiber, epicore dried yeast extract, carob fiber, sorghum bran, or any fiber from Table 1.

In some embodiments, a composition described herein may comprise one or more probiotics. In some cases, the composition comprises one or more of Lactobacillus paracasei, Lactobacillus rhamnosus, Bacillus coagulans and Saccharomyces boulardii. In some cases, the composition comprises one or more microbes from Table 2. In some embodiments, a composition for promoting health in a subject comprises a probiotic. In some cases, the composition comprises an amount of a probiotic from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of probiotic of about 1%, 2%, 3%, 4%, 5%, 6%, 7%, 8%, 9%, 10%, 11%, 12%, 13%, 14%1, 5%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of a probiotic of at least about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of a probiotic of less than about 1%, 2%, 3%, 4%, 5%, 6%, 7%, 8%, 9%, 10%, 1%, 2%, %13%, %14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises yellow kiwi fruit extract. In some cases, the composition comprises an amount of yellow kiwi fruit extract from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of yellow kiwi fruit extract of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of yellow kiwi fruit extract of at least about 1%, 2%3, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of yellow kiwi fruit extract of less than about 1%, 2%, 3%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises turmeric. In some cases, the composition comprises an amount of turmeric from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of turmeric of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of turmeric of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of turmeric of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises green kiwi fruit extract. In some cases, the composition comprises an amount of green kiwi fruit extract from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of green kiwi fruit extract of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of green kiwi fruit extract of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight. In some cases, the composition comprises an amount of green kiwi fruit extract of less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight.

In some embodiments, a composition for promoting health in a subject comprises lychee fruit extract. In some cases, the composition comprises an amount of lychee fruit extract from about 5% to 95%, from about 5% to about 90%, from about 5% to about 80%, from about 10% to about 50%, from about 10% to about 40%, from about 10% to about 30%, or from about 20% to about 25% by weight. In some cases, the composition comprises an amount of lychee fruit extract of about 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9%, 10%, 11%, 12%, 13%, 14%, 15%, 16%, 17%, 18%, %19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% lychee fruit extract by weight. In some cases, the composition comprises an amount of lychee fruit extract of at least about 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% lychee fruit extract by weight. In some cases, the composition comprises an amount of lychee fruit extract of less than about 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4% 3.5% 3.6%, 3.7% 3.8%, 3.9% 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28% 29%, 30%, 31%, 32%, 33%, 34%, 35%, 36%, 37%, 38%, 39%, 40%, 45%, or 50% lychee fruit extract by weight.

In some embodiments, a composition for promoting health in a subject comprises one or more of curcumin, kiwi fruit extract, green tea polyphenol, berberine, green coffee bean powder, and blueberry extract powder. In some embodiments, a composition for promoting health in a subject comprises one or more components selected from Table 3.

### Compositions

In some cases, the composition comprises potato starch and locust bean gum. In some cases, the composition comprises potato starch, locust bean gum, and one or more of oat bran, Galacto-oligosaccharides, apple fiber, orange fiber, barley bran, oat fiber, pea fiber, chia, kudzu, beta glucan, partially hydrolyzed guar gum, gum Arabic, and soluble corn fiber. In some cases, the composition comprises potato starch, locust bean gum, and oat bran. In some cases, the composition comprises potato starch, locust bean gum, oat bran, and one or more of Galacto-oligosaccharides, apple fiber, orange fiber, barley bran, oat fiber, pea fiber, chia, kudzu, beta glucan, partially hydrolyzed guar gum, gum Arabic, and soluble corn. In some cases, the composition comprises 23% potato starch, 1% locust bean gum, 27.5% oat bran, and one or more of Galacto-oligosaccharides, apple fiber, orange fiber, barley bran, oat fiber, pea fiber, chia, kudzu, beta glucan, partially hydrolyzed guar gum, gum Arabic, and soluble corn fiber. In some cases, the composition comprises potato starch, locust bean gum, oat bran, Galacto-oligosaccharides and apple fiber. In some cases, the composition comprises 23% potato starch, 19% locust bean gum, 27.5% oat bran, 20% Galacto-oligosaccharides and 10.5% apple fiber by weight. In some cases, the composition comprises 23% potato starch, 19% locust bean gum, 27.5% oat bran, 10.5% Galacto-oligosaccharides and 20% apple fiber by weight. In some cases, the composition comprises potato starch, locust bean gum, oat bran, orange fiber and barley bran. The composition of claim 21, comprising 23% potato starch, 19% locust bean gum, 27.5% oat bran, 20% orange fiber and 10.5% barley bran by weight. In some cases, the composition comprises potato starch, locust bean gum, oat bran, apple fiber and oat fiber. The composition of claim 23, comprising 23% potato starch, 19% locust bean gum, 27.5% oat bran, 20% apple fiber and 10.5% oat fiber by weight. In some cases, the composition comprises resistant potato starch, locust bean gum, oat bran, barley bran and pea fiber. The composition of claim 25, comprising 23% potato starch, 19% locust bean gum, 27.5% oat bran, 20% barley bran and 10.5% pea fiber by weight. In some cases, the composition comprises potato starch, locust bean gum, oat bran, kudzu and chia. The composition of claim 27, comprising 23% potato starch, 19% locust bean gum, 27.5% oat bran, 20% chia and 10.5% kudzu by weight.

In some cases, the composition comprises potato starch, locust bean gum, and beta-glucan. In some cases, the composition comprises resistant potato starch, locust bean gum, and beta-glucan, and one or more of Galacto-oligosaccharides, partially hydrolyzed guar gum, pea fiber, gum Arabic, soluble corn fiber, oat bran, apple fiber, orange fiber, barley bran, oat fiber, chia, or kudzu. In some cases, the composition comprises 23% potato starch, 19% locust bean gum, and 27.5% beta glucan. In some cases, the composition comprises 23% potato starch, 19% locust bean gum, and 27.5% beta glucan, and one or more of Galacto-oligosaccharides, partially hydrolyzed guar gum, pea fiber, gum arabic, soluble corn fiber, oat bran, apple fiber, orange fiber, barley bran, oat fiber, chia, or kudzu. In some cases, the composition comprises potato starch, locust bean gum, beta-glucan, Galacto-oligosaccharides and partially hydrolysed guar gum. In some cases, the composition comprises 23% potato starch, 19% locust bean gum, 27.5% beta glucan, 23% Galacto-oligosaccharides and 7.5% partially hydrolyzed guar gum. In some cases, the composition comprises 23% potato starch, 19% locust bean gum, 27.5% beta glucan, 15.5% Galacto-oligosaccharides and 15% partially hydrolyzed guar gum. In some cases, the composition comprises potato starch, locust bean gum, beta-glucan, Pea fiber and partially hydrolysed guar gum. In some cases, the composition comprises 23% potato starch, 19% locust bean gum, 27.5% beta glucan, 20% Galacto-oligosaccharides and 10.5% pea fiber. In some cases, the composition comprises potato starch, locust bean gum, beta-glucan, pea fiber and gum arabic. In some cases, the composition comprises 23% potato starch, 19% locust bean gum, 27.5% beta glucan, 20% gum Arabic and 10.5% pea fiber. In some cases, the composition comprises potato starch, locust bean gum, beta-glucan, soluble corn fiber and partially hydrolysed guar gum. In some cases, the composition comprises 23% potato starch, 19% locust bean gum, 27.5% beta glucan, 20% soluble corn fiber and 10.5% partially hydrolysed guar gum. In some cases, the composition comprises potato starch, locust bean gum, beta-glucan, chia and kudzu. In some cases, the composition comprises 23% potato starch, 19% locust bean gum, 27.5% beta glucan, 20% chia and 10.5% kudzu.

In some embodiments, a composition described herein may comprise one or more probiotics. In some cases, the composition comprises one or more of Lactobacillus paracasei, Lactobacillus rhamnosus, Bacillus coagulans and Saccharomyces boulardii.

In some cases, the composition comprises potato starch, locust bean gum, concentrated β-glucan, Galacto-oligosaccharides and partially hydrolyzed guar gum.

In some embodiments, a composition described herein may comprise one or more probiotics. In some cases, the composition comprises one or more of Lactobacillus paracasei, Lactobacillus rhamnosus, Bacillus coagulans and Saccharomyces boulardii. In some embodiments, a composition described herein may comprise one or more biologically active compounds. In some cases, the composition comprises one or more of yellow kiwi fruit extract, turmeric extract, green kiwi fruit extract, and lychee fruit extract by weight. In some cases, the composition comprises about 34% yellow kiwi fruit extract, 28% turmeric extract, 34% green kiwi fruit extract, and 2.8% lychee fruit extract by weight. In some cases, the composition comprises about 40% yellow kiwi fruit extract, 17% turmeric extract, 40% green kiwi fruit extract, and 3% lychee fruit extract by weight.

In some embodiments, a composition described herein is provided as a tablet, capsule, powder blend, gummy, nutritional bar, or liquid formulation. In some embodiments, the composition may comprise one or more excipients.

The compositions for use in accordance with the present invention can be formulated in a conventional manner using one or more physiologically acceptable carriers or excipients. Agents used in the formulations and their physiologically acceptable salts and solvates can be prepared for administration by various methods. In an exemplary embodiment, administration of the formulations is oral.

For oral administration, the formulations can take the form of, for example, tablets, capsules, powders, gummies, nutritional bars or liquid formulations. Tablets or capsules may be prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. In one embodiment, the liquid preparations can be formulated for administration with fruit juice, e.g., apple juice. Such liquid preparations can be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). Other suitable non-aqueous vehicles may include neuroprotective foods, e.g., fish oil, flaxseed oil, etc. The preparations can also contain buffer salts, flavoring, coloring and sweetening agents as appropriate.

Preparations for oral administration may be provided as a unit dosage form, for example, as tablets, capsules, etc. These can be presented in blister packs or in multi-dose containers. Preparations for oral administration can also be suitably formulated to give controlled release of the active compound.

### Biological Functions

In some embodiments, the compositions described herein promote health, for example gut health, glucose regulation, satiety, immune function or lipid control. Promoting health may comprise improving or maintaining one or more indicators of health, or improving a symptom. Promoting health may comprise increasing production of at least one short-chain fatty acid in the digestive tract of the subject. Examples of short-chain fatty acids include, but are not limited to, acetate, propionate, or butyrate. In some cases, promoting health comprises increasing abundance of one or more keystone microbial species in the digestive tract of the subject. Examples of keystone microbial species include, but are not limited to, Faecalibacterium prausnitzii, Akkermansia muciniphila, Collinsella aerofaciens, Eubacterium hallii, Bacteroides thetaiotaomicron, Roseburia hominis, or Eubacterium rectale. In some cases, promoting health comprises decreasing abundance of one or more pathogenic microbial species in the digestive tract of the subject. Examples of pathogenic microbial species include but are not limited to Clostridioides difficile, Shigella sonnei, Escherichia coli, Campylobacter, Shigella flexneri, Shigella dysenteriae, Shigella boydii, Campylobacter gracilis, Citrobacter freundii, or Citrobacter braakii. In some cases, promoting health comprises treating antibiotic caused dysbiosis. Promoting health may also comprise increasing a Bacteroidetes to Firmicutes ratio in the digestive tract of the subject, and/or increasing secretion of GLP-1, PYY, or IL-10 in the digestive tract of the subject. Promoting health may comprise increasing expression of one or more of Gcg, Pcsk1, Pyy, Tlr4, Muc1, and Muc2 in the digestive tract of the subject.

In some cases, a composition described herein promotes health in a healthy subject. In some cases, a composition described herein promotes health in a subject with type 2 diabetes. In some cases, a composition for promoting health in a subject with type 2 diabetes comprises potato starch, locust bean gum, concentrated β-glucan, Galacto-oligosaccharides and partially hydrolyzed guar gum. In some cases, a composition for maintaining glucose regulation in a subject with type 2 diabetes comprises potato starch, locust bean gum, concentrated β-glucan, partially hydrolyzed guar gum, and pea fiber. In some cases, a composition for maintaining glucose regulation in a subject with type 2 diabetes comprises potato starch, locust bean gum, concentrated β-glucan, partially hydrolyzed guar gum, and pea fiber. In some cases, a composition for promoting immune function in a subject with type 2 diabetes comprises potato starch, locust bean gum, concentrated β-glucan, soluble corn fiber, and partially hydrolyzed guar gum. In some cases, a composition for promoting lipid regulation in a subject with type 2 diabetes comprises potato starch, locust bean gum, concentrated β-glucan, chia and kudzu.

In some cases, the composition comprises about 40% yellow kiwi fruit extract, 17% turmeric extract, 40% green kiwi fruit extract, and 3% lychee fruit extract by weight and promotes health in a healthy individual. In some cases, a composition comprises about 40% yellow kiwi fruit extract, 17% turmeric extract, 40% green kiwi fruit extract, and 3% lychee fruit extract by weight, and promotes health in a subject with type 2 diabetes.

In some embodiments, the present disclosure provides methods for promoting health in a subject, the method comprising administering a composition described herein. Promoting health may comprise improving or maintaining health, for example improving or maintaining gut health, glucose regulation, satiety, immune function or lipid control. In some cases, promoting health comprises increasing production of at least one short-chain fatty acid in the digestive tract of the subject. Examples of short-chain fatty acid include acetate, propionate, and butyrate. In some cases, promoting health comprises increasing abundance of one or more keystone microbial species in the digestive tract of the subject. Keystone microbial species may include Faecalibacterium prausnitzii, Akkermansia muciniphila, Collinsella aerofaciens, Eubacterium hallii, Bacteroides thetaiotaomicron, Roseburia hominis, and Eubacterium rectale. In some cases, promoting health comprises decreasing abundance of one or more pathogenic microbial species in the digestive tract of the subject. Pathogenic microbial species include Clostridiosis difficile, Shigella sonnei, Escherichia coli, campylobacter, Shigella flexneri, Shigella dysenteriae, Shigella boydii, Campylobacter gracilis, Citrobacter freundii, and Citrobacter braakii. Promoting health may also comprise treating antibiotic caused dysbiosis. In some cases, promoting health comprises increasing a Bacteroidetes to Firmicutes ratio in the digestive tract of the subject. Promoting health may comprise increasing secretion of GLP-1, PYY, or IL-10 in the digestive tract of the subject, or increasing expression of Gcg, Pcsk1, Pyy, Tlr4, Muc1, or Muc2 in the digestive tract of the subject. The subject may be a healthy subject or a subject with type 2 diabetes.

### EXAMPLES

### Example 1: Cholesterol Adsorption Assessment

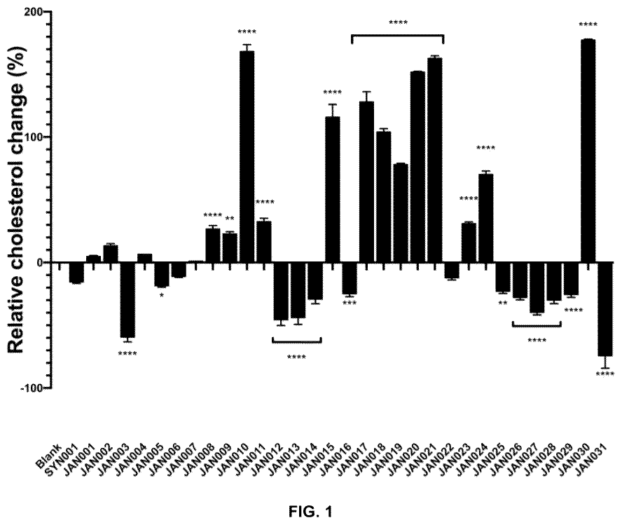

The fibers were assessed for effect on cholesterol adsorption using a Total Cholesterol Assay Colorimetric Kit (Cell Biolabs). 60 mg of fiber was mixed to homogeneity with 5 ml of water. As a source of both cholesterol and cholesteryl esters, egg yolks were separated from the whites and diluted with water in a 1:10 weight/volume ratio. This was mixed well to full emulsion. Enough HCl was added to set the emulsion to a pH of 3.5. 5 ml of the emulsion was added to each fiber mixture and vortexed thoroughly to mix. A blank reaction tube included 5 ml of yolk emulsion to 5 ml of water. Following an incubation of 2 hours at 37° C., samples were centrifuged at 2000×g for 10 minutes at room temperature. The supernatant was poured off into a new set of conical vials. Fractions were normalized by adding enough 1× Total Cholesterol Assay working diluent to make the solution up to 14 ml. Cholesterol standards provided in the kit and unknown samples were then plated out on Costar Clear Polystyrene 96-Well Plates (Corning). The assay master mix, consisting of Cholesterol Esterase, Cholesterol Oxidase, HRP, and Colorimetric Probe (Cell Biolabs), was added to the wells. The plate was covered in foil and set in a 37° C. incubator for 45 minutes with constant mixing at 150 RPM. The plate was then measured for absorbance at 560 nm using a SpectraMax iD3 Plate Reader (Molecular Devices). The results are shown in FIG. 1.

The first step is mimicking the chemical and physical conditions of the upper human gastrointestinal tract prior to reaching the colon, where only the Microbiota Accessible Carbohydrates (MACs) would be exposed to the colonic microbial community for fermentation. Across all experiments, distilled water was used as blank and Fructooligosaccharide (Sigma Aldrich) was used as a positive control.

### Example 2: Upper GI Digestion Mimicking

The Upper GI digestion mimicking was performed as below:
1. 12.5 grams of samples (food/fiber/prebiotic) weighed in a beaker.
2. 150 mL distilled water added to the dry powder sample.
3. The mix was placed on a heating stirring plate and stirred while gradually increasing the temperature to the boiling point.
4. The mixture was boiled for 20 minutes.
5. The mixture was cooled to 37° C. and approximately 4 mL of 1 M HCl (Hydrochloric acid) solution added to bring down the pH to 2.5.
6. 5 mL of 100 mg/mL pepsin (Sigma Aldrich #P-7000) dissolved in 50 mM HCl (Hydrochloric Acid) was added and mixed for 30 min at 37° C. with a constant stir bar.
7. 25 mL of 0.1 M Sodium Maleate buffer (pH=6, containing 1 mM CaCl₂)) was added and approximately 10 mL of 1 M NaHCO₃ (Sodium Bicarbonate) to bring pH down up to 6.9.
   0.1 M Sodium Maleate buffer (pH=6.0): Dissolve 11.6 g of maleic acid (Sigma #M0375) in 800 mL of distilled water and adjust the pH to 6.0 with 4 M NaOH. Add 0.735 g of CaCl₂·2H₂O (Calcium chloride, dihydrate) and 0.2 g of sodium azide and dissolve. Adjust the volume to 1 L. (This solution is stable for 12 months at 4° C.). (Solution is light-sensitive so cover with aluminum foil.)
   1 M NaHCO, (sodium bicarbonate): Dissolve 84.01 g of NaHCO₃ (Sigma #S5761) in 800 mL of distilled water and adjust the volume to 1 L.
8. 25 mL of 125 mg/mL pancreatin (Sigma #P-7545) dissolved in sodium maleate buffer was added and 1 mL of amyloglucosidase (3260 U/ml, Megazyme #E-AM6DF) and the reaction was incubated at 37° C. for 6 h under constant stirring.
9. Samples were freeze-dried and ground at slow speed for uniform mixing.

After upper gastrointestinal tract digestion mimicking, samples were tested for neutral monosaccharide composition analysis to identify the polysaccharide structures and linkages present in each prebiotic fiber.

Six sets of fermentation experiments were conducted, three with healthy stool samples (identified on the basis of lack of any predisposed metabolic syndrome or similar health conditions), and another three samples from clinically diagnosed Type 2 Diabetic individuals. The samples were diluted in the ratio 1:100 with gut mineral media under anaerobic condition in the Coy Vinyl anaerobic glove box containing the anaerobic gas mix (10% H2, 5% CO2, and balanced N2). For all the experiments, the gut mineral media contained trace elements: 8.0 mM NaCl, 6.3 mM KCl, 3.3 mM urea, 3.3 mM NH4Cl, 0.7 mM Na2SO4, 40 mM sodium phosphate buffer (pH 7.0), 1 mg resazurin, 0.25 g/L cysteine HCl, 333 μM CaCl₂), 492 μM MgCl2, and 1×P1 metals.

A time zero sample was collected from the dilute stool samples. The fiber samples were then treated with these diluted stool samples in Balch tubes, sealed with rubber caps and aluminum seals, before moving the tubes from the anaerobic chamber into a shaker incubator set to 37° C. with constant shaking. Samples were collected at 24 hr and 48 hr time points from the time of inoculation. The stool donors had been on habitual diets and had not taken any antibiotics for the past 6 months prior to study initiation.

During the 24 hr and 48 hr time point, gas produced was quantified via a graduated syringe. This method was performed by passing a needle through a rubber stopper and measuring the increase in the pressure of the overhead space in the tube. Gas production can be a surrogate marker of microbial fermentation and is confirmatory that the fermentation has occurred successfully without gas leaks or the introduction of oxygen into the Balch tubes.

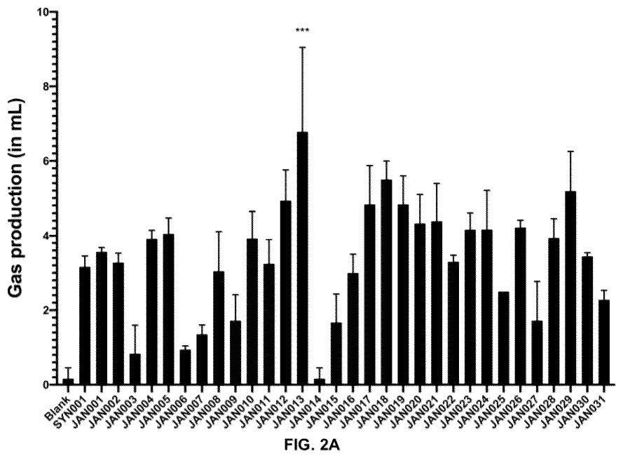

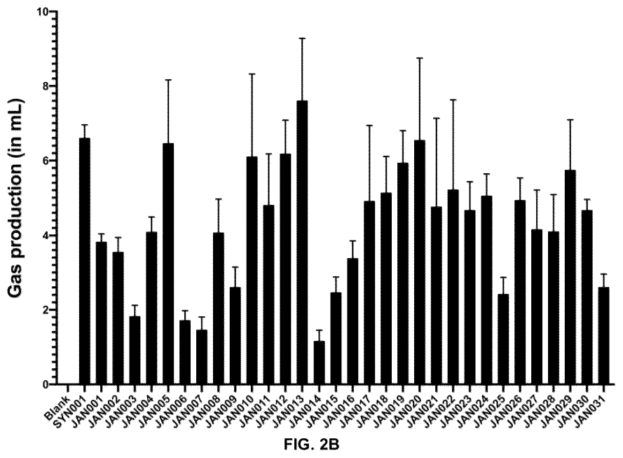

FIG. 2A shows the increase in the total gas production after the in vitro fermentation assay. Gas production is a surrogate marker that fermentation successfully occurred in the tubes without atmospheric air leaking in. The main gases produced during the process of in vitro fermentation are ammonia, hydrogen sulfide, methane, and carbon dioxide. In FIG. 2A and FIG. 2B, which shows average gas production for healthy and Type 2 Diabetes stool fermentation respectively, the Blank (No Carbon substrate) shows the least gas production. Minimal fermentation might have occurred due to some remaining prebiotic fractions that could be present in the fecal slurry. Amongst the 31 fibers, JAN014 shows the least gas production for both healthy and Type 2 stool fermentation experiments. Reduced gas production is observed for high cellulose-containing fibers: JAN003 (41.48%), JAN006 (76.4%), JAN007 (36.11%), and JAN014 (90.23%) for both Healthy and Type 2 Diabetes donors as shown in FIG. 2A and FIG. 2B respectively. Contrarily, JAN013 and JAN029 have higher gas production, which means higher fermentation.

The total gas production for Type 2 Diabetes Donors is higher than that of healthy donors as shown in FIG. 2A and FIG. 2B. This could indicate a high abundance of gas-producing bacterial strains present in the gut microbiota of Type 2 Diabetes donors. JAN010, JAN023, JAN024, JAN026, and JAN030 are fiber candidates with high gas producing scores across most donors. This is attributed to the ideal range of gas production by these prebiotic candidates which is an indication of fermentation success rate as well as minimal detrimental effects under in vivo conditions.

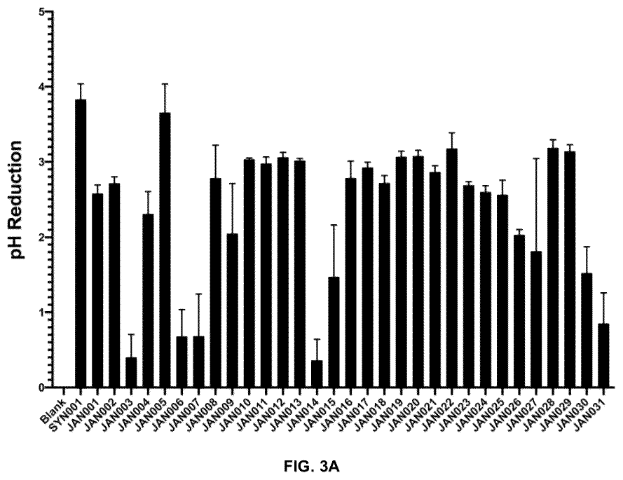

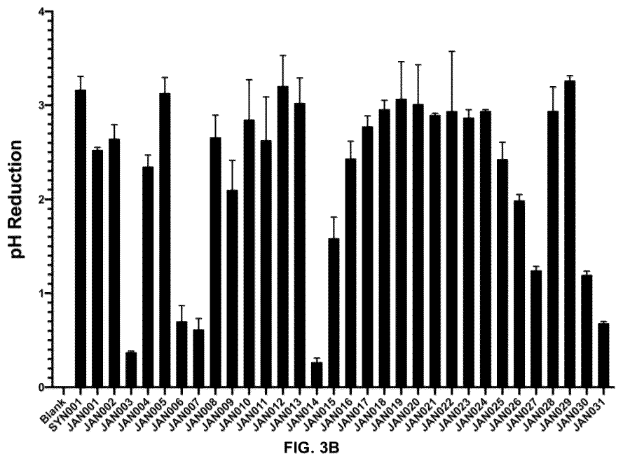

pH was measured for each sample replicate at the 24 hr and 48 hr time point. For measuring the pH, the fermentation supernatant was transferred to a separate 15 mL falcon tube and measured using a pH meter. FIG. 3A and FIG. 3B show the reduction in pH measured using a pH meter at post-fermentation time points across healthy and Type 2 diabetes donors respectively. Reduction in pH was calculated by subtracting the pH of samples from that of the pH of gut mineral media (pH of gut mineral media=7). The lower the pH, the higher the reduction from that of the baseline, which means the higher the levels of acid production, and a greater extent of fermentation. Various acidic metabolites are produced during fermentation resulting in the reduction of pH. For graphical representation in FIG. 3A and FIG. 3B, all the samples were normalized to blank samples (no carbon source). SYN001 (Sigma-Aldrich Fructooligosaccharide) and JAN005 (Food Grade Inulin) showed the highest reduction in pH from that of baseline, as low as 3.2 from 7.0 for both. The other high acid producers include JAN010, JAN011, JAN012, JAN013, JAN022, JAN028, and JAN029 for both healthy and Type 2 diabetes donors. One striking difference between healthy and Type 2 diabetes pH reduction is the overall lower acid production for Type 2 Diabetes fermentation samples compared to that of healthy donors. The fermentation supernatant replicates with a high reduction in pH were then subjected to the absolute quantification using GC-FID. A strong correlation between increased gas production and reduction in pH was observed for fibers JAN003, JAN006, JAN007, JAN013, and JAN014, suggesting that higher gas production is related to a higher reduction in the pH and vice versa for both population groups. SYN001 which is used as a positive control for fermentation in all our studies has shown higher pH reduction than all January prebiotic fibers for the healthy group (FIG. 3A). However, some fiber candidates JAN012 and JAN029 show higher average pH reduction for Type 2 Diabetes Donors as shown in FIG. 3B. This difference in the rate and extent of fermentation between the two groups may indicate differential microbial metabolic capacities between them.

The fermentation supernatants were semi-quantified using Thin Layer Chromatography as a preliminary confirmatory test for short-chain fatty acid production. This was run on a TLC Silica gel 60 F254 plates with a 5 component mobile phase consisting of acetone, chloroform, ethanol, water, and formic acid. Plates were dried and dipped into an indicator solution of methyl red and bromophenol blue in 70% methanol. Images were taken immediately following air dry at peak indicator development. TLC plates were semi-quantified using densitometric analysis (ImageJ). Fermentation replicates were picked based on consistent and higher quantities of short-chain fatty acids visible on the TLC.

Acetic, propionic, butyric, isovaleric, and isobutyric acids were quantified by Gas Chromatography—Flame Ionization Detector (GC-FID). Fermentation supernatants were homogenized using MP Bio FastPrep for 1 minute at 4.0 m/s. 5 M HCl was added to acidify fecal suspensions to a final pH of 2.0. Acidified fecal suspensions were incubated and centrifuged at 10,000 rpm to separate the supernatant. Fermentation supernatants were spiked with 2-ethylbutyric acid for a final concentration of 1 mM which is used as an internal standard for each sample. Extracted short-chain fatty acids were stored in 2 mL GC vials, with glass inserts. short-chain fatty acids were detected using gas chromatography (Thermo Trace 1310) coupled with a Flame Ionization Detector (Thermo Scientific). A Thermo TG-WAXMS column (30 m, 0.32 mm, 0.25 μm) was used.

The following settings were used for detection:

Flame Ionization Detector:
Temperature: 240° C.
Hydrogen: 35.0 ml/min
Air: 350.0 ml/min
Makeup gas (Nitrogen): 40.0 ml/min

### Inlet

Carrier pressure: 225 kPa
Column flow: 6.00 ml/min
Purge flow: 5.00 ml/min
Split flow: 12.0 ml/min
Temperature: 200° C.
Splitless time: 0.75 min

### Oven

Temperature Gradient: 100-180° C.
Gradient time: 10.0 min
   The short-chain fatty acid concentrations were measured in mM.

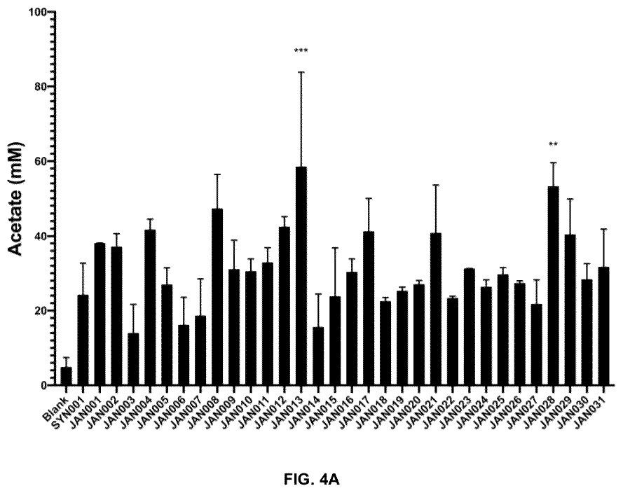

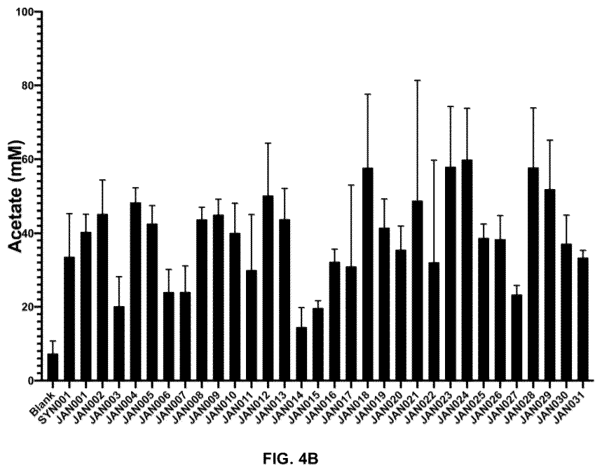

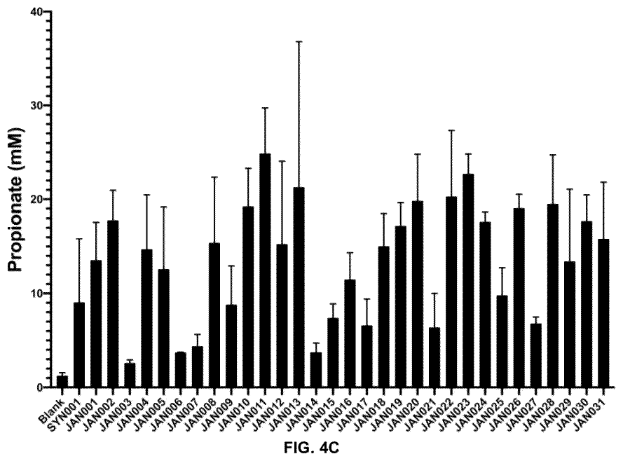

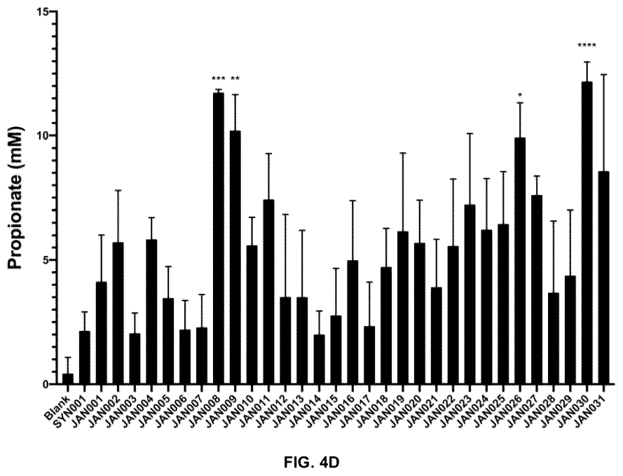

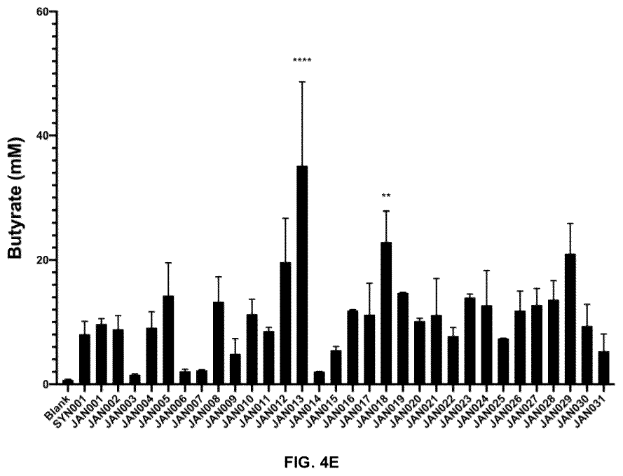

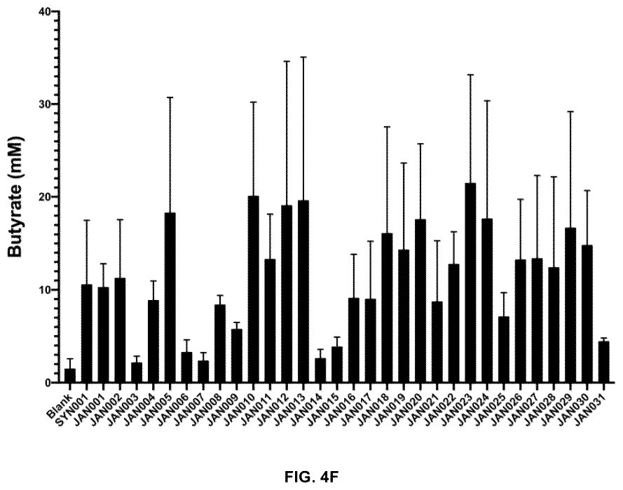

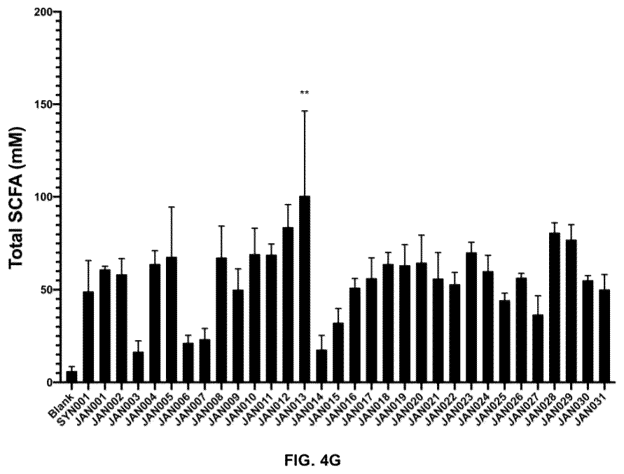

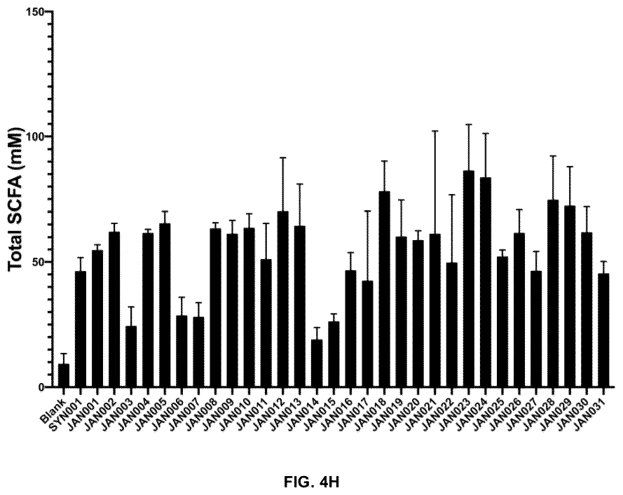

FIGS. 4A, 4C, 4E, and 4G show acetate, propionate, butyrate, and total short-chain fatty acids production in mM using the GC-FID absolute quantification method for healthy donors. FIGS. 4B, 4D, 4F, and 4H show acetate, propionate, butyrate, and total short-chain fatty acids production in mM using the GC-FID absolute quantification method for Type 2 diabetes donors. short-chain fatty acids are organic acids with 1 to 6 carbon molecules and are primary products of non-digestible carbohydrate fermentation in the human intestinal lumen. short-chain fatty acids could contribute to 5% to 15% of the total caloric requirement of humans. Short-chain fatty acids from the high fermenting fibers were roughly produced in the ratio 3:1:1, acetate:propionate:butyrate.

FIGS. 4A and 4B show the total acetate production after fermentation in mM for healthy and Type 2 diabetes donors respectively. Acetate may have the potential to directly bind to G-Protein Coupled Receptor (GPR), GPR43 (FFAR3), and GPR41 (FFAR2), these receptors may be involved in various metabolic activities relating to insulin sensitivity. Acetate is also an intermediate product for butyrate production via enzymatic activity of butyryl CoA. JAN013 and JAN028 have significantly higher acetate producing capacity compared to that of SYN001 as shown in FIG. 4A. JAN018, JAN023, and JAN024 have higher acetate producing capacity in Type 2 Diabetes donors compared to that of healthy donors.

Propionate is another major short-chain fatty acids produced during the process of fermentation in the human colon, thus it is considered in FIGS. 4C and 4D (healthy and Type 2 diabetes donors respectively). High acetate producing fibers are also seen to be high propionate producing across both groups. JAN003, JAN006, JAN007, and JAN014 are both low acetate and propionate producing fibers for both groups, whereas JAN013 and JAN028 remain on the high end for acetate and propionate for healthy donors only. Fibers like JAN011, JAN022, and JAN023 are specifically high propionate producing fibers for healthy donors. Interestingly, JAN011 and JAN022 both belong to the same category of fibers, guar gum, one being native form and the other being partially hydrolyzed form. It can be said from this finding that guar gum polysaccharides have a high propionate producing capacity. JAN008, JAN009, and JAN030 are significantly higher propionate production compared to that of SYN001 for Type 2 diabetes donors. Similar to acetate, propionate production potential is also different for fibers between healthy and Type 2 diabetes donors. Propionate may reduce antibiotic caused dysbiosis in the human gut under in vitro conditions. Propionate production may correlate with increased PYY and GLP-1 secretion, and may also increase IL-10 secretion.

Almost 90% of butyrate is metabolized in the colonic epithelial cells as the preferred source of energy. Butyrate may have anti-pathogenic activity by reducing Salmonella in the cecum. It may also inhibit the inflammatory response through NFkB inhibition in Crohn's disease. Butyrate production may be directly related to GLP-1 secretion which plays a critical role in managing blood glucose levels. This correlation is seen in the high butyrate production for JAN013 as shown in FIGS. 4E and 4F. JAN013 and JAN029 are the common butyrogenic fibers for both the healthy and Type 2 diabetes groups. JAN013 and JAN018 are significantly high butyrate-producing fibers compared to SYN001 for the healthy group. More fibers are seen to be butyrogenic in the Type 2 diabetes group, even though not significantly higher than SYN001 due to large error bars for inter-donor variance. Total short-chain fatty acid was calculated by adding all three individual short-chain fatty acids (acetate, propionate, and butyrate) as shown in FIGS. 4G and 4H for healthy and Type 2 diabetes groups respectively. For total short-chain fatty acids, there are multiple prebiotic candidates that show higher Total short-chain fatty acids than that of SYN001. Some of the striking ones are JAN012, JAN013, JAN028, JAN029 for the healthy group, even though JAN013 is the only one significantly higher than SYN001. For the Type 2 diabetes group, there are prebiotics with higher efficacy than that of SYN001. All three short-chain fatty acids acetate, propionate, and butyrate may have a synergistic effect on the host response markers.

Shallow Shotgun metagenomics was performed for each 48 hr fermentation sample. Samples were extracted with MO Bio PowerSoil Pro (Qiagen) automated for high throughput on QiaCube (Qiagen), with bead beating in 0.1 mm glass bead plates. Samples were quantified with Quant-iT Picogreen dsDNA Assay (Invitrogen). Libraries were prepared with a procedure adapted from the Nextera Library Prep kit (Illumina). Libraries were sequenced on an Illumina NovaSeq using single-end 1×100 reads (Illumina). DNA sequences were filtered for low quality (Q-Score<30) and length (<50), and adapter sequences were trimmed using cutadapt. Fastq files were converted to a single fasta using shi7. Sequences were trimmed to a maximum length of 100 bp prior to alignment. DNA sequences were aligned to a curated database containing all representative genomes in RefSeq for bacteria with additional manually curated strains (Venti). Alignments were made at 97% identity against all reference genomes. Every input sequence was compared to every reference sequence in CoreBiome's Venti database using full gapped alignment with BURST. Ties were broken by minimizing the overall number of unique Operational Taxonomic Units (OTUs). For the taxonomy assignment, each input sequence was assigned the lowest common ancestor that was consistent across at least 80% of all reference sequences tied for the best hit. Samples with fewer than 10,000 sequences were also discarded. OTUs accounting for less than one-millionth of all species-level markers and those with less than 0.01% of their unique genome regions covered (and <1% of the whole genome) were discarded. The number of counts for each OTU was normalized to the average genome length. Count data were then converted to relative abundance for each sample. The normalized and filtered tables were used for all downstream analyses. Phylum level data was used to calculate the Bacteroidetes/Firmicutes ratio. Data were further analyzed to elucidate significance in the change of relative species level populations. Z-scores were assigned and plotted internally via gplots: Heatmap.2 in R.

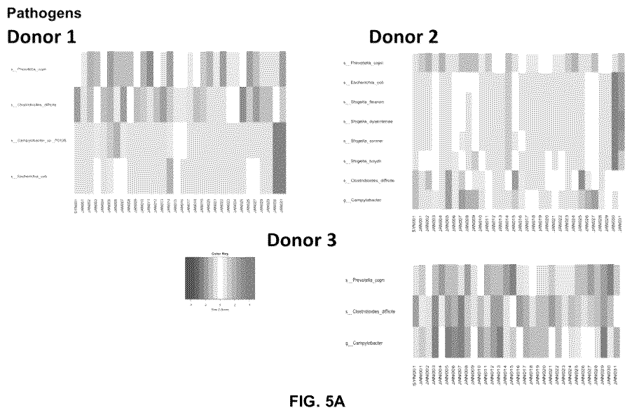

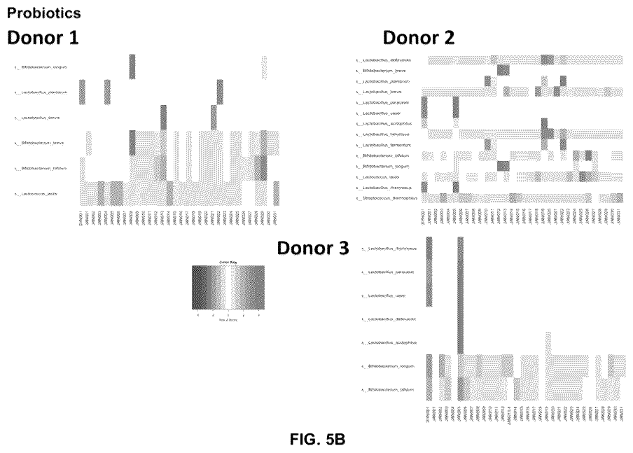

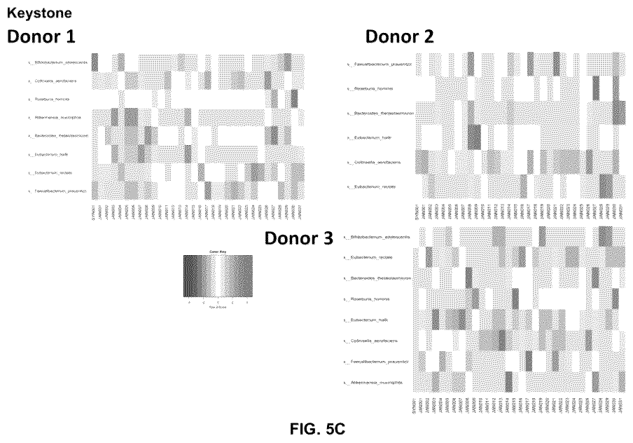

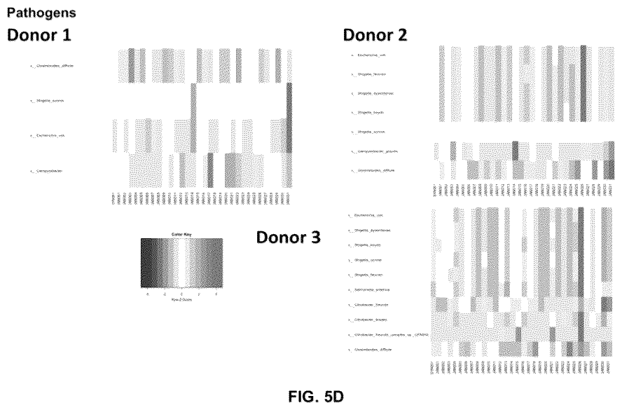

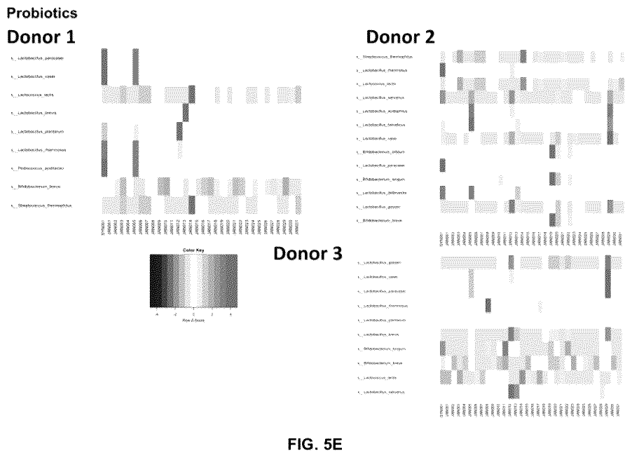

FIG. 5 presents the specific interactions of fibers with bacterial species with a high potential to affect the host from which they were derived. These heatmaps present the strength that these fibers interact specifically with different clades of bacteria. Every donor has a specific set of bacteria within the keystone, probiotic, and pathogenic groups that are due to the variance in the microbiome between people. However, there are trends showing that certain fibers can target niches or clades of bacteria, for a certain translatable function. For example, in FIG. 5A Prevotella copri and other Prevotella bacteria are specifically upregulated greatly by JAN011. With a z-score of at least of +2 across all donors (p<0.045, two-tailed). This shows that JAN011 might be a fiber to avoid due to the capacity for introducing a specific carbohydrate source for this possible pathogen. Another example of a detrimental effect would be JAN030 and JAN031 upregulating Shigella and E. coli with z-scores of +4 (p<0.0001). This trend is confirmed with the Type 2 Diabetic samples seen in FIG. 5D. In diabetic donors 1 and 2, both E. coli and Shigella were greatly upregulated by JAN030 and JAN031. When looking at a personalized approach, these fibers may be flagged as fibers to avoid in donors that have these populations of bacteria in their gut. Comparing a more positive effect, Clostridium difficile, Campylobacter, and Shigella like species are downregulated by JAN013. This fiber has a z-score of at least −2 (p<0.045, two-tailed) across all donors. This is again confirmed in the diabetic cohort, where Campylobacter and C. difficile are also downregulated by JAN013.

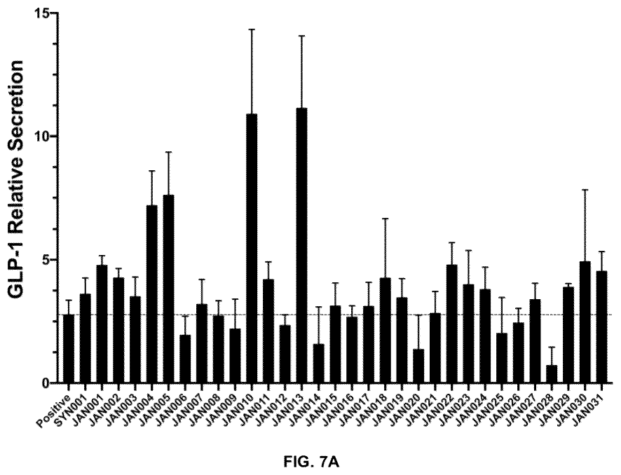

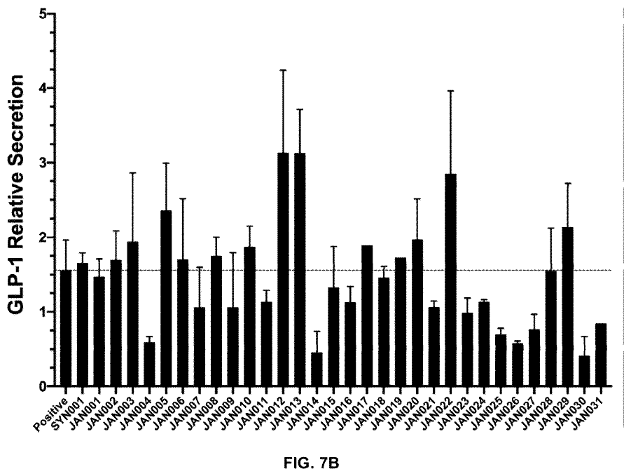

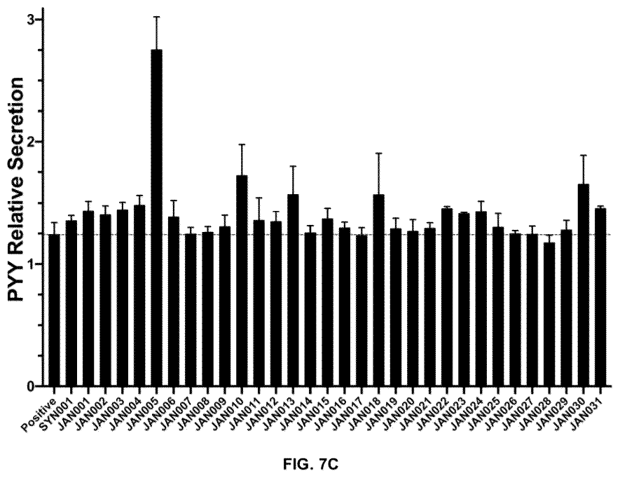

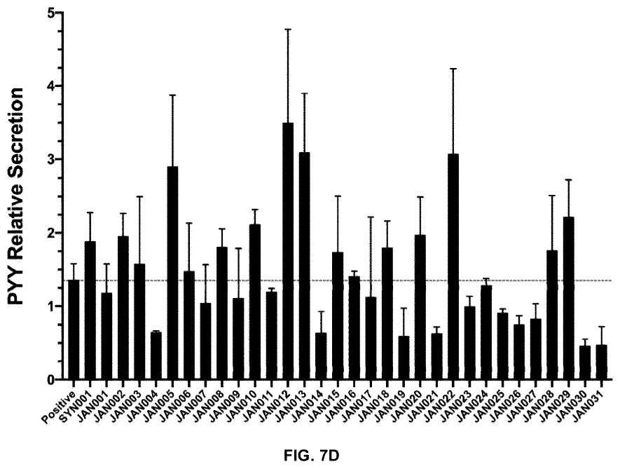

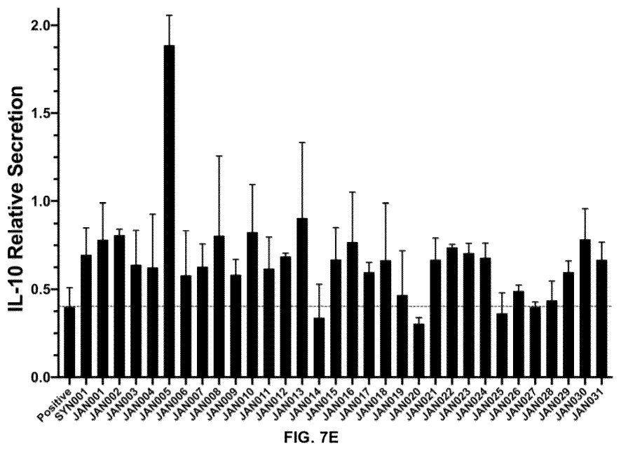

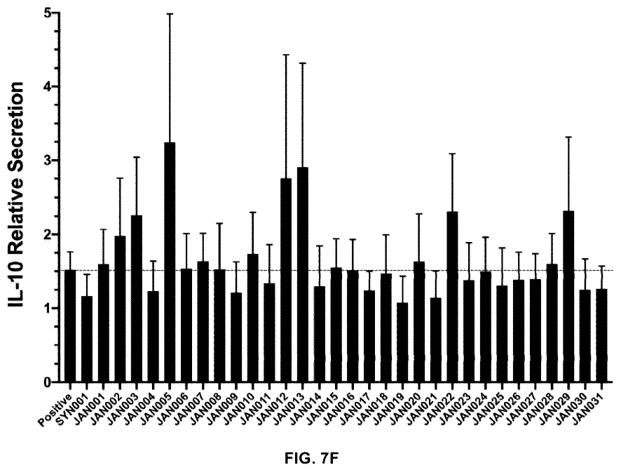

FIG. 5B shows the capacity of the different fibers to improve probiotic populations that reside in the donor's fecum. JAN005 presents a highly significant effect on Lactobacilli with a z-score of +4 (p<0.001). Donor 1 does not show this effect because this donor lacks the same Lactobacillus species that reacted to JAN005 in Donor 2 and 3. This suggests that even in the genus of Lactobacillus there is still variation amongst species for reactivity with certain fibers. Furthermore, comparing SYN001, a chemically pure derivative of JAN005, there is a lesser effect on these populations. This highlights the importance that fibers with similar structures may not have the same effect on the bacterial population which could contribute to lower downstream effects. This is further evident in comparing the efficacy of JAN005 to SYN001 in Butyrate production (FIG. 4C), GLP-1, PYY, and IL-10 secretion (FIG. 7). Comparing the diabetic donor fermentation samples shows the same specificity for fiber from different probiotic species. As seen in FIG. 5E JAN005 had high specificity for targeting a number of Lactobacilli. Outside of JAN005, JAN012 and JAN013, both tuber based starch-rich fibers, promote Bifidobacterium populations across the healthy and Type 2 Diabetic cohort. Bifidobacterium tends to be acetogenic and generally promotes gut health with clinical results. Another interesting find is that while JAN014 lags behind most fibers in terms of short-chain fatty acid production and Host Hormone Secretion, there is a specificity to promote Lactococcus lactis and Streptococcus thermophilus, which may be important probiotic species.

Keystone bacteria are the microbiota that are associated with the relief of metabolic syndrome symptoms through a review of clinical trial data. Therefore, these are the designated species believed to drive mechanisms for anti-diabetic and gastrointestinal tract health benefits. Considering FIG. 5C, keystone species are highly specific for fibers. When looking at Eubacterium hallii, across all donors JAN008 and JAN009 (z>+2, p<0.1) upregulate while JAN019 and JAN020 (z<−2, p<0.1) downregulate. Similarly, there is a trend with Roseburia hominis, which was greatly upregulated with JAN009, JAN0027, and JAN030. In Donor 3 the relative abundance values increased across all fibers meaning that while JAN027 was not the most significant mover of R. hominis it still did increase the population. This is again confirmed by the Donor 1 in type 2 diabetic cohort in FIG. 5F. Faecalibacterium prausnitzii, one of the validated key players in improving diabetic symptoms, consistently improves with JAN017 (z>+4 Donor 3, z>+3 Donor 1) and JAN021 (z>+3.5 Donor 2 and 3, z>+2 Donor 1). However, there are a number of other fibers that do also improve the population of F. prausnitzii. Both these high effectors and medium effectors are considered, as again the composite scores across the microbial species for each fiber are additive.

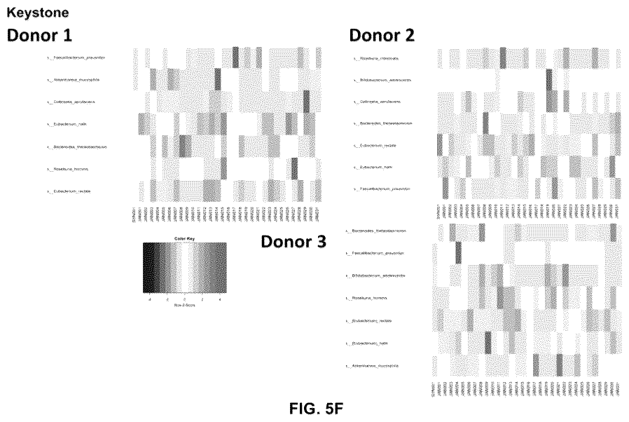

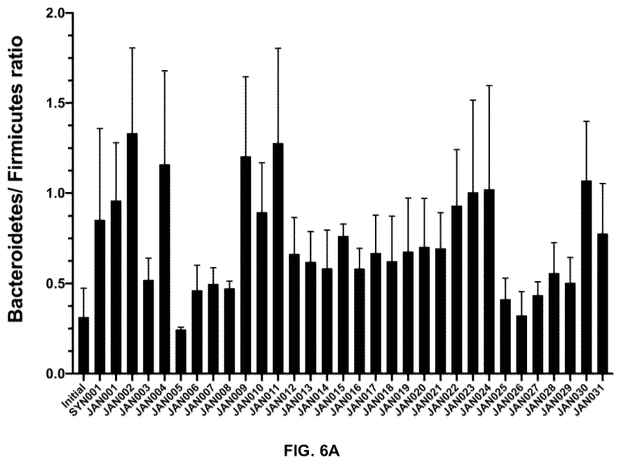

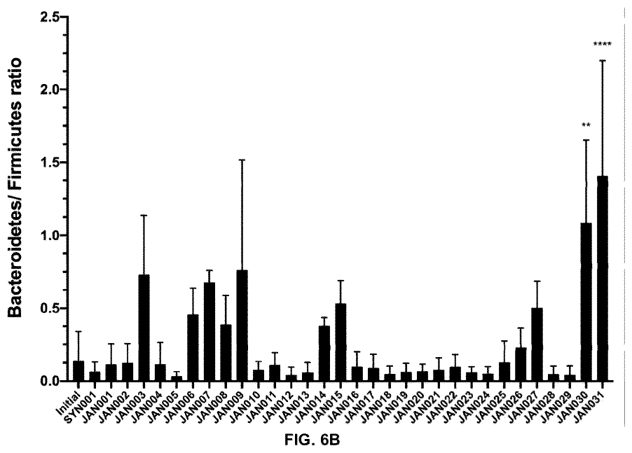

The Bacteroidetes/Firmicutes ratio may be a biomarker for improvement of BMI, weight loss, and is seen to be significantly lower in diabetic fecal samples. As such, this is a consideration to review. As seen in FIG. 6A, all JAN fibers except for JAN005 and JAN026 show improvements in this ratio. As this is averaged data across all donors, these values are not significant. However, when looking at the difference between the Initial samples versus the fiber samples, there is significant improvement across the whole cohort (2-way ANOVA, p<0.0001). Therefore, fiber was seen to drive a positive effect of inducing an increase in the Bacteroidetes population. This is within the expectation as Bacteroidetes metabolize and grow on these complex carbohydrates. Additionally, while the Firmicutes population decreases relative to the Bacteroidetes, key Firmicutes players like Roseburia and Faecalibacterium that convert acetate into the desired metabolite of butyrate, are conserved among the rest of the cohort. JAN002, JAN009, and JAN011 showed the greatest improved effect on the population across donors nearly 5-fold greater than the initial samples. In the Type 2 Diabetic Donors, seen in FIG. 6B, two fibers: JAN030 (p<0.01) and JAN031 (p<0.0001) stood out as statistically significant when averaging all three donors (Tukey's multiple comparison test). While not statistically significant in the average of donors, JAN003, JAN007, and JAN009 were consistently higher than SYN and the initial samples, meaning the anti-diabetic effect of increased Bacteroidetes was prominent in these fermentation conditions. When comparing the healthy individuals to the Type 2 Diabetic individuals, there is a clear difference in the initial sample and the breadth of effect with the fiber fermentation conditions. In FIG. 6A, most fiber fermentation conditions improve the ratio, while only a select few improve the ratio in the type 2 diabetics in FIG. 6B. Interestingly, fibers that react well with type 2 diabetics, do not improve the Bacteroidetes/Firmicutes ratio in healthy individuals. This is evident in JAN003, JAN007, and JAN027. While the final diabetic average ratios are around the same as the healthy donors (˜0.5 B/F), the delta from the initial sample is quite amplified in the diabetic donors being 6 fold greater vs only a 0.6 fold improvement in the healthy donors. Therefore, the potential to improve the Bacteroidetes/Firmicutes ratio by generic fibers is greater in the healthy donors than the type 2 diabetics, but the potential for specific personalized fibers to greatly improve this ratio may be greater for the diabetics.

Resected human colonic primary cells expressing a mixture of enterocytes, enteroendocrine, and goblet cells, was grown in specialized Colonic Epithelial Cell Medium and passaged to reach a cell density of 300,000 cells/ml. Cells were then plated on 6-well plates and grown to confluence. Cells were treated with fermentation supernatants for a 1-hour incubation at 37° C. Butyrate was used as a positive control at a 200 mM concentration. The fermentation blank, a sample with only fermentation media and donor fecal mixture, was used as the negative control. An inulin fermentation supernatant also acted as a second reference positive control. Supernatants were extracted for downstream secreted hormone analysis. Cells were washed using Dulbecco's Phosphate Buffer Solution and lysed followed by RNA extraction using the RNeasy Mini Kit (Qiagen).

Three colonic epithelial hormones and cytokines, GLP-1 (total), PYY, and IL-10, were quantified via electrochemiluminescence with the U-PLEX assay platform (Mesoscale Discovery, MSD). U-plex plates were made a day before running the samples. Biotinylated capture antibodies were conjugated to 3 assigned linkers (GLP-1 Total: 1, PYY: 3, and IL-10: 10). These were incubated at room temperature for 30 min followed by the addition of stop buffer incubated at another 30 min. Capture antibodies with linkers were combined and plated on the U-plex plate. These were incubated for 1 hour at room temperature followed by storage at 4° C. overnight. The following day, samples and standards were diluted with Diluent 13 supplemented with aprotinin. Samples were incubated on the U-plex plate for 2 hours, followed by a 1-hour incubation with the SULFO-Tag Detection antibody. The plate was primed with MSD Gold Read Buffer A and read on the MESO QuickPlex SQ 120. Custom assay plate analysis was implemented by assigning GLP-1 Total assay to spot 1, PYY to spot 3, and IL-10 to spot 10. Using the Discovery Workbench software, 4-parameter standard curves were derived for each hormone of interest from standard cocktails provided by MSD. Unknown sample concentrations were extrapolated from these curves. A ratio was calculated of each sample compared to the blank cell culture control and reported as relative protein secretion.

A gene expression-based assay was designed as an orthogonal approach to assay the colonic cells. cDNA was synthesized using qScript™ XLT cDNA SuperMix (QuantaBio). Randomized primers, oligodTs, and RNase H(+) derivatives of MMLV reverse transcriptase were used in the mastermix to convert lysate RNA to the first-strand cDNA. A PCR program of 5 minutes at 25° C., 30 minutes at 42° C., 5 minutes at 85° C., and held at 4° C. was performed in a MaxyGene Thermal Cycler II (Axygen). TaqMan™ Gene Expression Assay (Life Technologies) primers targeting: Gcg, Pcsk1, Pyy, Tlr4, Muc1, and Muc2 were used for this assay. FAM MGB-NFQ was used as the reporter/quencher for this assay. Taqman Fast Advanced Master Mix (Applied Biosystems) and cDNA were plated on 384 well PCR plates (ABgene) and run on the Quantstudio 6 (Applied Biosystems). Gapdh was used as a reference gene for delta delta Ct relative gene expression. All samples were compared to the negative cell culture control. 2(ΔΔCt) values were then averaged amongst replicate plates to be reported as relative expression.

FIG. 7 presents the reaction of primary colonic epithelial cells to the fermented fibers. GLP-1 is induced greatly by JAN010 (Tukey's multiple comparison test to Positive, p<0.01) and JAN013 (Tukey's multiple comparison test to Positive, p<0.0001) (FIG. 7A). When looking at each donor specifically these are significantly different than both the butyrate positive control as well as the fiber control of SYN001. These fibers induce a 10 fold increase in GLP-1 secretion compared to the fermentation blank, which represents the donor's capacity to induce GLP-1 with their current diets. Therefore, fiber supplementation has the potential to greatly improve the glucose regulation and bowel movement functions of GLP-1 produced by the colon. Similar to the healthy donor samples, JAN013 stands out again in eliciting GLP-1 production in type 2 diabetic samples (FIG. 7B). When averaged across donors this effect is not considered significant but when looking at the individual donors, across the board JAN013 is highly significant (p<0.0001) over both the butyrate positive control and the SYN control. JAN012 is quite similar in polysaccharide structure to JAN013 since both are tubers and contain 16.4% and 12.5% resistant starch respectively. Resistant starches act as strong producers of short-chain fatty acids through fermentation. This shows a clear correlation between the fermentation capacity of certain fibers to produce Butyrate and the other short-chain fatty acids to trigger GLP-1 across both healthy and diabetic cohorts.

JAN010 and JAN013 also had an impact on PYY secretion. FIG. 7C shows that the enteroendocrine cells have specificity for the metabolites released from the fermentation of JAN005. JAN005 induces a 3 fold increase in PYY compared to the capacity of existing metabolites in all donor's fecum. Most of the other fibers did generally increase secretion above baseline, however, most were not significant compared to the 200 mM Butyrate and SYN001 conditions. As mentioned previously, even though JAN005 and SYN001 have the same functional carbohydrate structure of Fructooligosacharrides, the source of JAN005 greatly impacts the fermentation capacity to drive short-chain fatty acids among other metabolites to induce a greater than 2 fold effect compared to SYN001. Similar fibers that induced GLP-1 in the diabetic cohort: JAN012, JAN013, JAN022, and JAN029, also induce PYY at nearly equivalent amounts over the basal condition. A potential reason for why there are more PYY spikes in the diabetic donors compared to the healthy donors could be that the basal levels of short-chain fatty acids, indoles, and proteins in the healthy donors already illicit a healthy satiety control baseline. Thus, only the JAN005 condition which induced a great increase in short-chain fatty acids was able to trigger this response than the pre-existing metabolites. Juxtaposing this result, there may be higher variance in the diabetic donor samples due to baseline deficiencies in these metabolites that promote satiety. Therefore, the conditions that improve fermentation and production of short-chain fatty acids and other beneficial microbial-derived metabolites can drive this amplified effect.

Finally, anti-inflammation is another factor to consider in terms of maintaining gut health and improving symptoms of a myriad of metabolic syndromes. IL-10 is also secreted by the enterocytes in colonic primary tissue and causes a cascade of responses intended for crosstalk with Tregs amplifying an anti-inflammatory signal. In this way, JAN005 is also a fiber candidate that causes downstream protection of the colonic lining (FIG. 7E). JAN005 had a 4 times greater capacity to trigger this IL-10 response compared to the Butyrate control (p<0.01). JAN010, JAN013, and JAN018 were highly significant compared to Butyrate and SYN001 controls with between 3 to 4 fold increased secretion in both Donor 1 and Donor 3 (p<0.0001). Since Donor 2 didn't have significance with these fibers, the average values are not clear of this trait. When looking at the diabetic donors in FIG. 7F we see a similar pattern with GLP-1 and PYY where JAN012, JAN013, JAN022, and JAN029 come up again. This may be due to the production of Butyrate as seen in FIG. 4F since these fibers are among the higher end of the butyrate-producing fibers. Butyrate is known to induce IL-10 production in the enterocytes that would in vivo trigger a macrophage IL-10 cascade. When looking at individual donors there is significance again between the positive control and SYN001 with p<0.0001 for these fibers with Type 2 Diabetes Donor 2 and p<0.01 for these fibers with Type 2 Diabetes Donors 1 and 3.

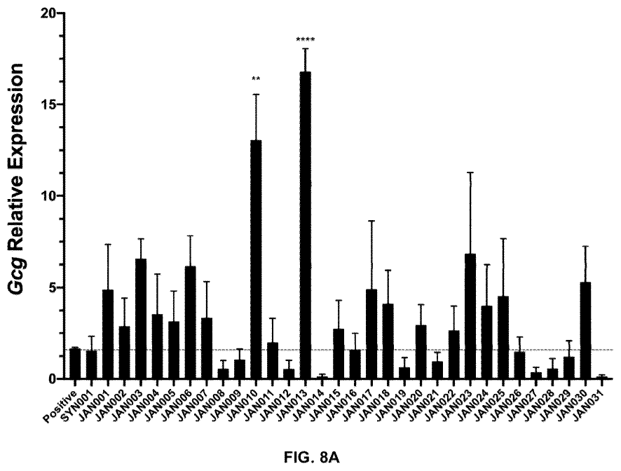

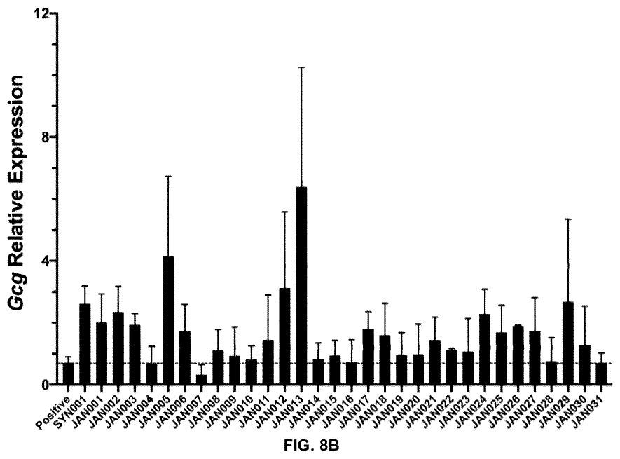

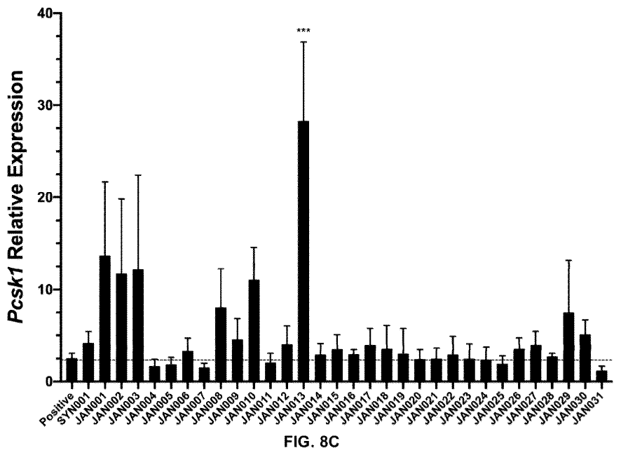

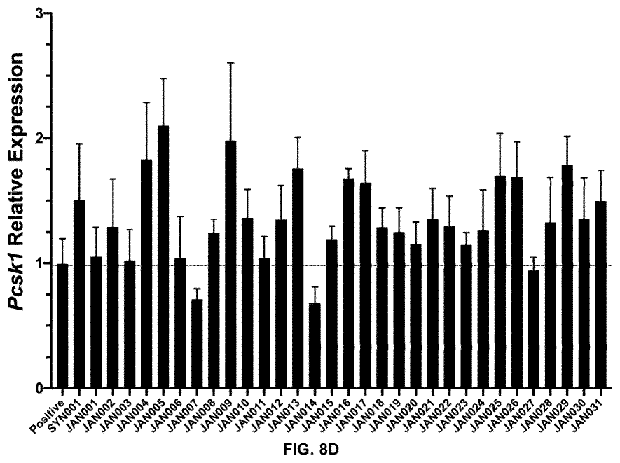

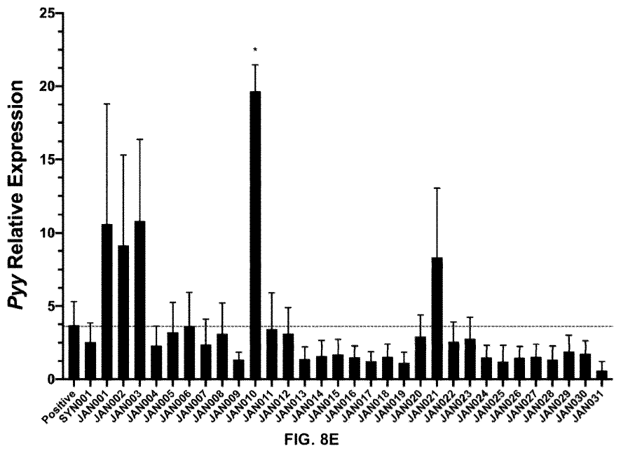

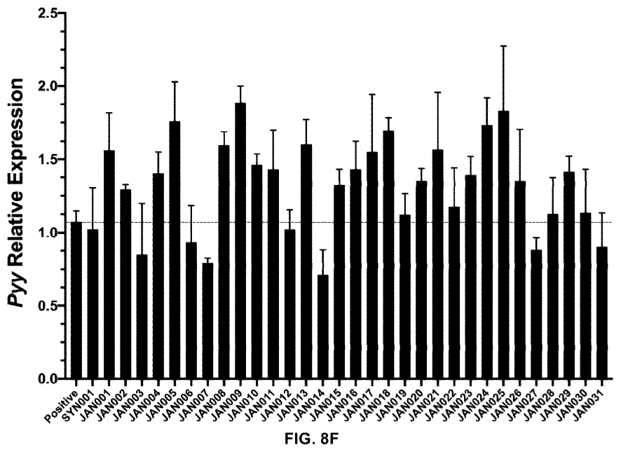

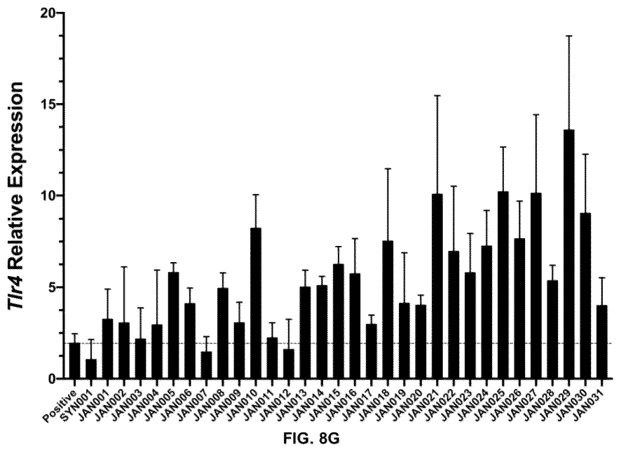

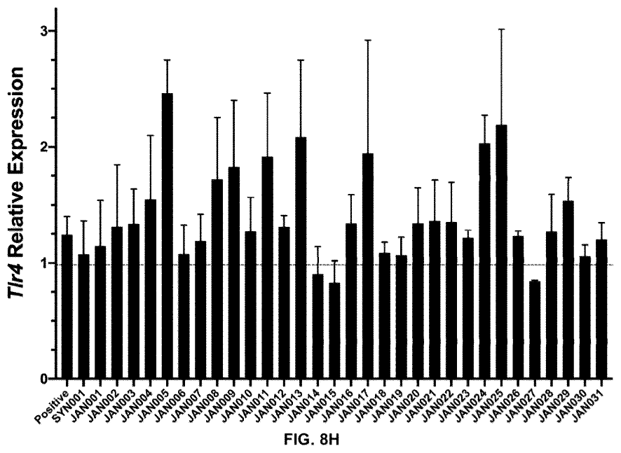

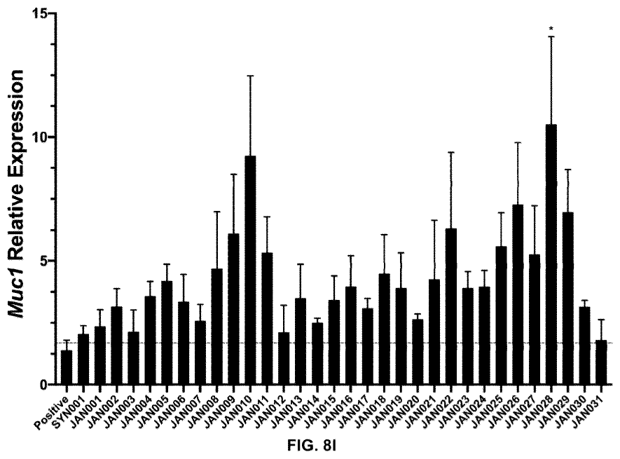

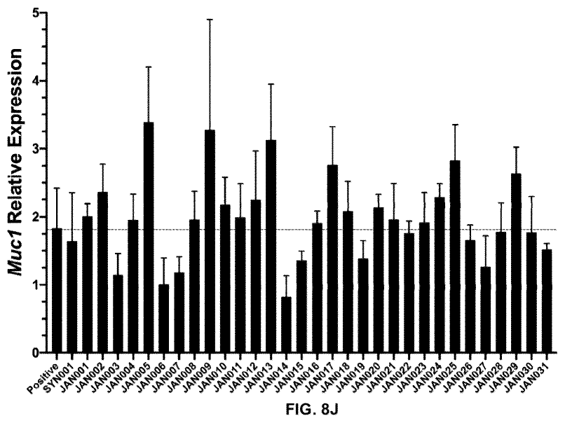

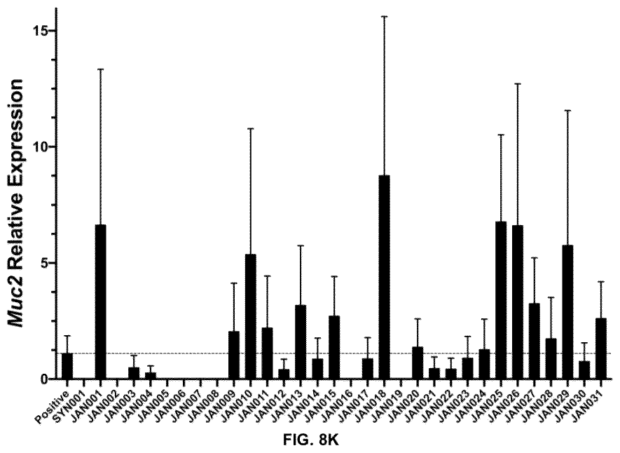

As an orthogonal measure of biological effect on the enterocytes and colonic L cells, gene expression was evaluated for the beneficial effects of fiber fermentation supernatants on human colonic tissue. In order to probe further into the strength of fibers to produce GLP-1, Gcg was a measure for potential de novo production of GLP-1 as it translates into Proglucagon, the precursor protein to GLP-1. As seen in FIG. 8A JAN010 (p<0.01) and JAN013 (p<0.0001) trigger the production of Gcg in a significant way over positive and SYN001 by 6 fold and 7 fold greater expression respectively. These findings were seen across the average of healthy donors, meaning that these two fibers may be important in translational beneficial host responses. These two fibers also trigger Gcg with the Type 2 diabetes Donor samples (FIG. 8B).

Another component of the glucoregulatory pathway, Pcsk1 codes for the proprotein convertase that converts proglucagon into GLP-1. In FIG. 8C, it is once again evident that JAN013 greatly affects the glucoregulatory pathway, since the relative expression of Pcsk1 is 15 fold greater than the positive control among the whole healthy cohort (p<0.001). Additionally, JAN001, JAN002, and JAN003 are 4 fold greater than SYN001, which is an effect driven by the high specificity with Healthy Donor 2 (p<0.0001). These three fibers are more whole food type fibers with a mixture of polysaccharide structures. The fermentation of more than one kind of polysaccharide and polyphenolic structures that come from mixed whole food may lead to improved glucose control and satiety. There are no significant fibers in Type 2 Diabetes for Pcsk1, but the same trends with JAN005 and JAN013 do appear as mild improvements of 2 fold greater than the positive controls in Donors 1 and 3 and 1.6 fold greater in Donor 2 (FIG. 8D). Pointing out the fibers that did not elicit a reaction, JAN007 and JAN014, performed worse than the positive controls. This trend is again seen in both the previous FIGS. 8A, 8B, and 8C exhibiting 2 fold lower expression than the positive controls. This may be due to the low short-chain fatty acids producing profile of JAN007 and JAN014 seen in FIG. 4H. A possible explanation for this would be the high cellulose contents in both of these fibers being 37% and 90% respectively. The higher percentage of cellulose means that the dietary fiber fermentation potential will be less than the other fibers.

In addition to considering PYY secretion as a measure of satiety, the relative expression of Pyy was also measured. Similar to measuring Gcg, Pyy serves as a biomarker for tracking the de novo production of this satiety hormone. In the healthy donor samples, FIG. 8E, there is a similar trend to the healthy donor Pcsk1 result, where JAN001, JAN002, and JAN003 are upregulated by 2.5 fold greater than the controls. Furthermore, Pyy is expressed less than Pcsk1 similar to how PYY is secreted at a lesser fold change compared to GLP-1. This interaction indicates that these fibers have the potential to increase both glucose regulation and satiety but not at a 1:1 ratio. However, not all fiber conditions elicit this correlation between GLP-1 and PYY. For example, JAN010 exhibits a unique specificity to induce satiety over glucose control. This is evident in healthy donor Pyy expression with a significant 4 fold greater effect than the positive controls (p<0.05) while Pcsk1 and Gcg remain lower at 2.5 fold and equal expression over control respectively. Interestingly, the opposite seems to be true about JAN013, which seems to reduce or inhibit the expression of Pyy by 4 fold, compared to the increase in Pcsk1 and Gcg by 15 fold each (p<0.0001). Thus, even though JAN010 and JAN013 produce great amounts of short-chain fatty acids and elicit GLP-1 secretion effects, it is possible that the structural biologies may promote de novo production of one hormone over the other. The Type 2 Diabetes cohort, in FIG. 8F, did not have the same robust induction of Pyy as did the healthy donors probably caused by the lack of underlying beneficial metabolites in the donors' fecal matter. However, interestingly, the standard deviation between donors was less, meaning that fibers that stand out like JAN008, JAN009, JAN018, and JAN024 could potentially act as positive inducing fibers across the diabetic cohort.

In terms of inflammation, Tlr4 is a key player in the protection of gut inflammation as well as a trigger for systemic inflammation. The IL-10-Tlr4 crosstalk that occurs between macrophages and enterocytes causes localized improvement in the gut as well as the potential to lower chronic inflammation systemically. Looking at FIG. 8G, a unique collection of fibers come up that haven't in the previous targets. JAN027 and JAN030 are beta-glucan based fibers that induce 5 fold greater levels of Tlr4 expression than the positive controls (p<0.0001 in Healthy Donor 2). JAN030 is a whole food version of beta-glucan that could also interact with the gut in a similar way to JAN027, albeit at a slightly lower effect. While these are new fibers that come up from this experiment, we still do see the fibers that are nicely correlated as top producers among the assays like JAN010, which is 4 fold greater than controls, and JAN029, which is 6.5 fold greater than controls (p<0.0001 in Donors 1 and 2 and p<0.05 in Donor 3). Interestingly, JAN014 actually induces somewhat of an effect in Tlr4, with a 2 fold expression over controls, compared to previous assays. However, this effect doesn't seem to translate into the Type 2 Diabetic samples in FIG. 8H, which follows the more typical pattern of JAN005 and JAN013 being the higher end of the fibers, albeit with only a 2 fold increased expression in these fibers than the positive control. JAN024 (p<0.0001 in Donor 1) and JAN025 (p<0.0001 in Donor 3) also are top hits in the diabetic samples with again around 2 fold increased expression. Again, beta glucan in JAN024 also acts to induce the immunity protective response of Tlr4 induction. Additionally, JAN025 is a soluble corn fiber designed for immune-modulating effects as well.

Another component that contributes to localized gut health and reducing inflammation is the secretion of mucin in the gut to improve the tight-barrier function of the colonic epithelia in the way of Mucin 1 and protect the epithelia from detrimental bacteria via Mucin 2. However, since these proteins are difficult to quantify due to issues with solubility, the expression of the corresponding genes needed to be considered. Thus, both Muc1 and Muc2 were considered as biomarkers for overall gut health speaking towards the reduction of inflammation in the gut. The only fiber condition that shows significant effect across all donors is JAN028 with a 6.7 fold relative expression compared to the positive controls (p<0.05) (FIG. 8I). This is one of the oligosaccharides that is easily fermented in the gut and has shown to produce short-chain fatty acids, although, a possible reason for its underperformance among other assays may be due to the lack of complexity compared to some of the other fibers that have the potential to produce a wide variety of microbial metabolites. However, since this effect was seen across all donors, the strength of JAN028 to improve the adhesion of gut epithelial cells to each other is quite evident. This effect was not seen in the diabetic donors portrayed in FIG. 8J, where JAN028 stayed at the baseline of the butyrate and SYN001 positive controls. JAN010 also does well in the healthy samples with a 9 fold induction of Muc1 compared to the butyrate positive control (p<0.0001 for all donors). Contrarily, the diabetic samples present a different set of fibers: JAN009, JAN013, JAN017, and JAN025.

While Muc1 shows an overall improved expression rate among most of the fibers, Muc2 is more specific and it seems that some fibers actually have the inhibitory capacity for its expression. This is evident in FIG. 8K, where SYN001 and JAN fibers 002-008 do not have the capacity to induce expression of Muc2 from the primary goblet cells in the tissue culture. Additionally, there are fibers that do produce less than the butyrate control most likely due to this inhibitory effect as well. Interestingly, JAN 002 (12.96% cellulose), JAN003 (41.84%), JAN004 (12.17%), JAN006 (76.4%), and JAN007 (37.11%) are whole food fibers that contain higher amounts of cellulose than the more pure JAN fiber samples. Unlike the healthy donor samples, the diabetic group seems to produce higher basal levels of Muc2, so that most of the fiber fermentation conditions can modulate the detectable levels of genes albeit at lesser fold change than with the healthy conditions (FIG. 8L). For example, JAN008 works 2.7 fold better than the positive controls in Donor 1 (p<0.0001), while JAN009 works 3 fold better than the controls in Donor 2 (p<0.0001). In the case of Donor 3, there are multiple fibers with highly significant fold expressions over the controls, where JAN004, JAN008, JAN010, JAN020, JAN021, JAN025, and JAN028 have 4 fold and above greater inductions than the positive controls (all p<0.0001).

Indoles are another product of microbial fermentation with dietary fibers. Total Indole content was measured in the highest and lowest efficacy prebiotic fibers using CellBiolabs Total Indole Assay kit (MET-5122). This assay was performed only for healthy donors as a proof of concept that there are other metabolites besides short-chain Fatty Acids that could play a crucial role in translating effect to host response. The protocol was followed exactly as provided with the kit.

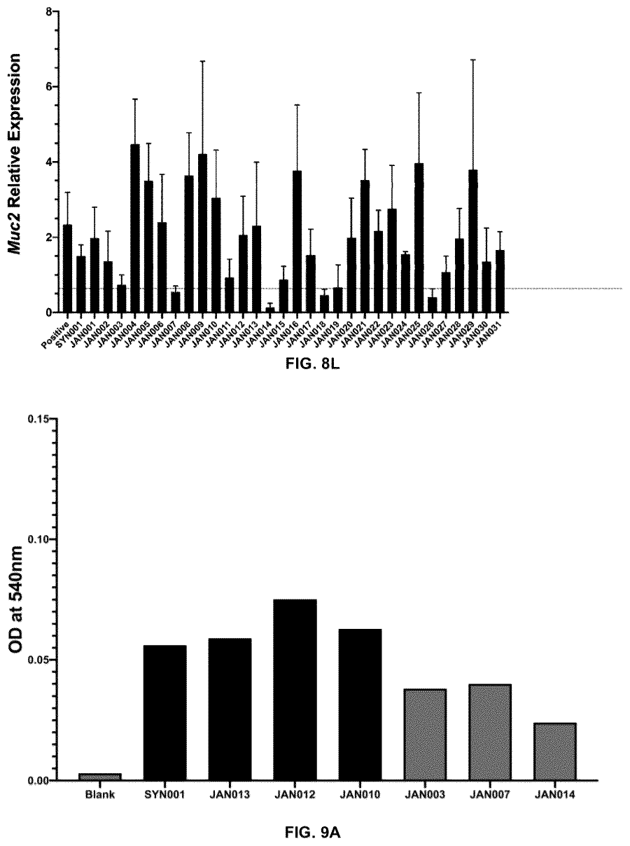

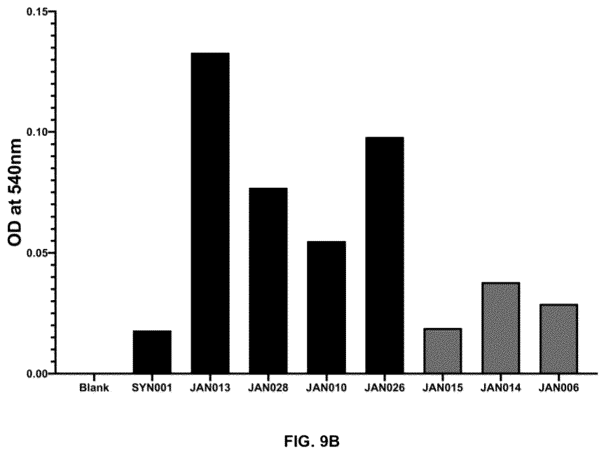

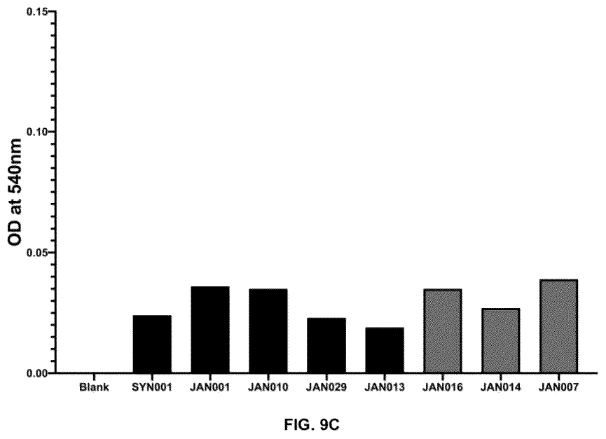

FIGS. 9A, 9B and 9C show indole quantification data from healthy donors 1, 2, and 3 respectively. We hypothesized that there are other metabolites beyond short-chain fatty acids driving the effect of the increased hormone secretion and gene expression in certain fibers like JAN013 and JAN010. FIG. 9A shows indoles production from Healthy donor 1 and it is evident that black bars have slightly increased amounts of total indoles compared to less efficacious grey bar fiber candidates. In FIG. 9B for Donor 2, we see that JAN013 and JAN026 are showing increasing indole contents compared to that of all other prebiotic fiber candidates. All the efficacious fibers for Donor 2 have shown increased indole contents compared to that of less efficacious fibers. The increased indole content in the fermentation supernatants could be driving the increased GLP-1 secretion. For donor 3 as shown in FIG. 9C, increased indole production was not seen in high efficacy fibers compared to that of low efficacy fibers.

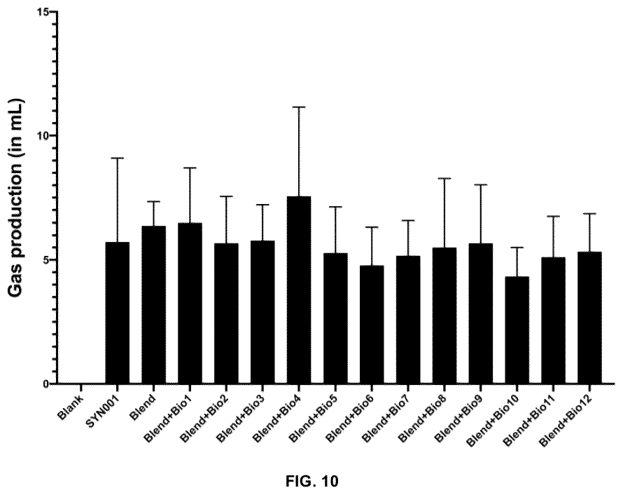

In FIG. 10, gas production is measured after fermentation with a prebiotic fiber blend in combination with bioactive polyphenol compounds. 20% of the total weight was assigned to gas production. Similar to that of prebiotic fibers, lower scores were assigned linearly with increasing gas production for the bottom 3 quartile, but a lower score to the top quartile due to potential bloating and gassing side effects. The initial sample is used as the blank. Blend+Bio 4 is the only Bioactive showing higher gas production as compared to that of Blend only. The hypothesis is that the amount of fermentation occurring in this mix is higher than the blend sample, which will be further confirmed with reduced pH and short-chain fatty acids production. In spite of low gas production in some Blend+Bioactive samples, like Blend+Bio8, Blend+Bio9 there is reduced pH compared to that of Blend only. As mentioned previously, some gases produced during fermentation could potentially be utilized by bacterial communities to further induce fermentation. Blend+Bio4, Blend+Bio8, and Blend+Bio9 showed increased pH reduction compared to that of Blend alone (FIG. 11). This suggests that there is evidence that polyphenolic bioactives in addition to prebiotic fibers can elicit a stronger fermentation response translating to increased metabolite production. This is confirmed with short-chain fatty acids quantification. Along with already known health benefits like being antioxidant and having antimicrobial properties of polyphenols, there is evidence polyphenols have fermentative activity.

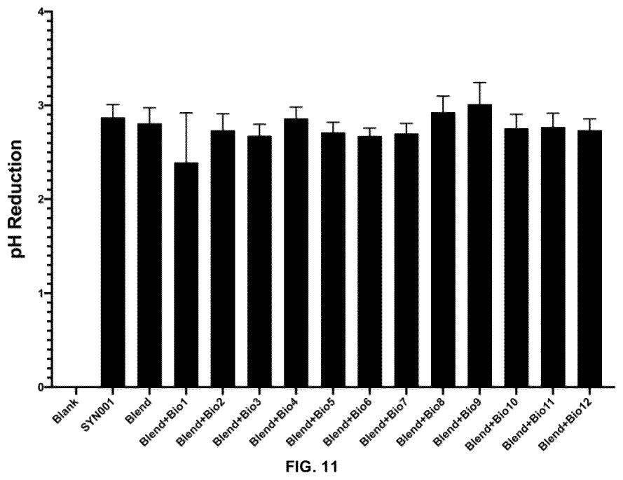

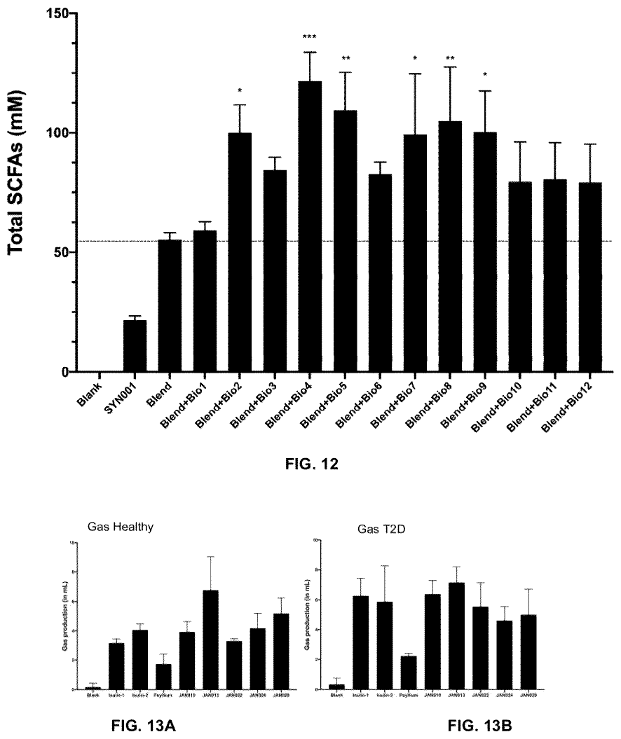

short-chain fatty acid analysis was semi-quantified using densitometric analysis via TLC. FIG. 12 shows that polyphenolic bioactives can contribute to the fermentation reactants and induce greater amounts of short-chain fatty acids. Bio4 is the top short-chain fatty acid producing bioactive compound that lines up well with the increased gas production presented in FIG. 12. Bio4 significantly increases the short-chain fatty acids concentration by 2.2 fold over the complete blend (p<0.001) and greater than 5 fold over SYN001 a commonly used dietary prebiotic fiber (p<0.0001). Additionally, Bio5 and Bio8 also cause an improved 2 fold response in short-chain fatty acids production over the complete blend (p<0.01). These are the top hits for bioactives that synergistically improve the functional output from the fermentation of the complete blend. Polyphenols in this way can act as beneficial metabolites or cofactors that help the gut microbiota to produce beneficial metabolites.

### Example 3: Comparison to Inulin and Psyllium Fiber

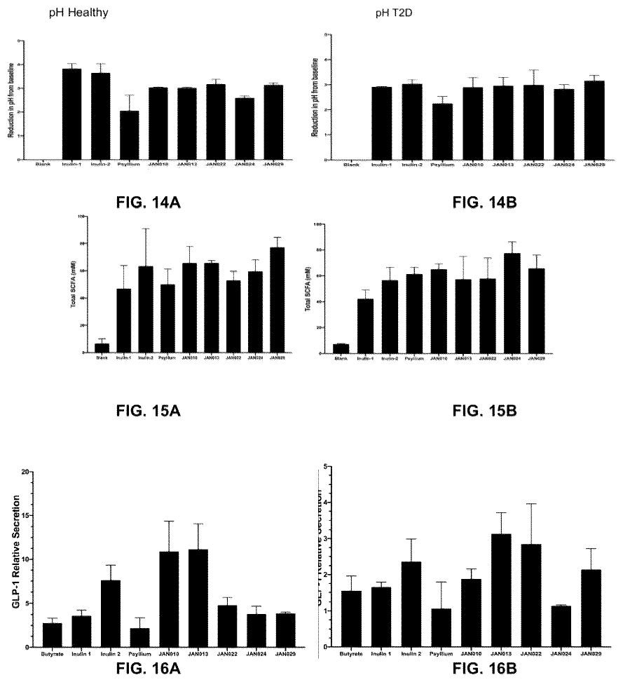

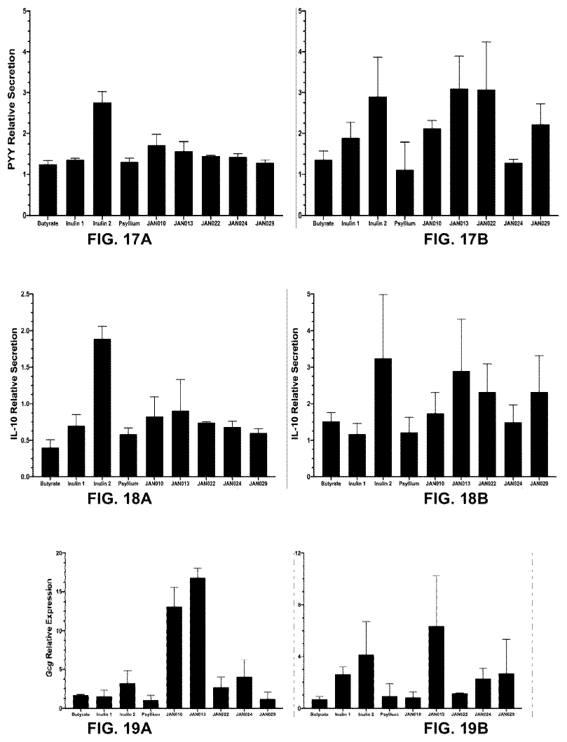

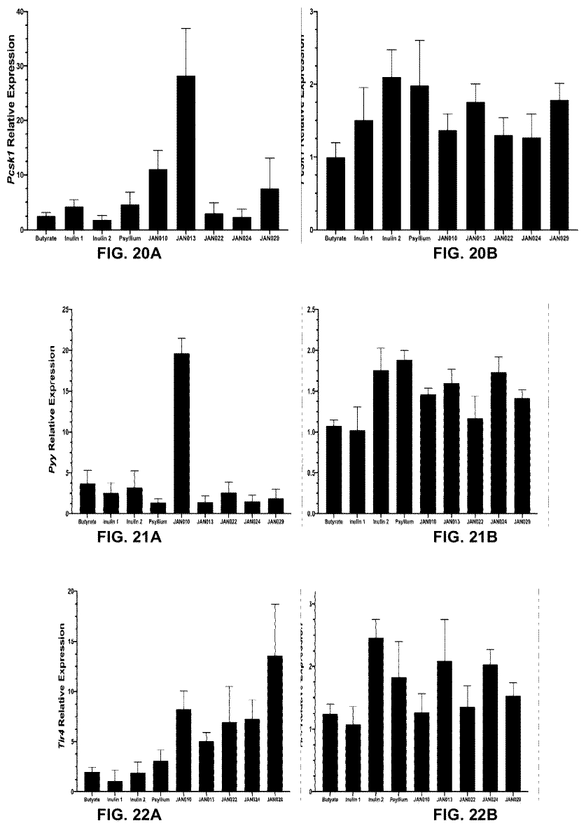

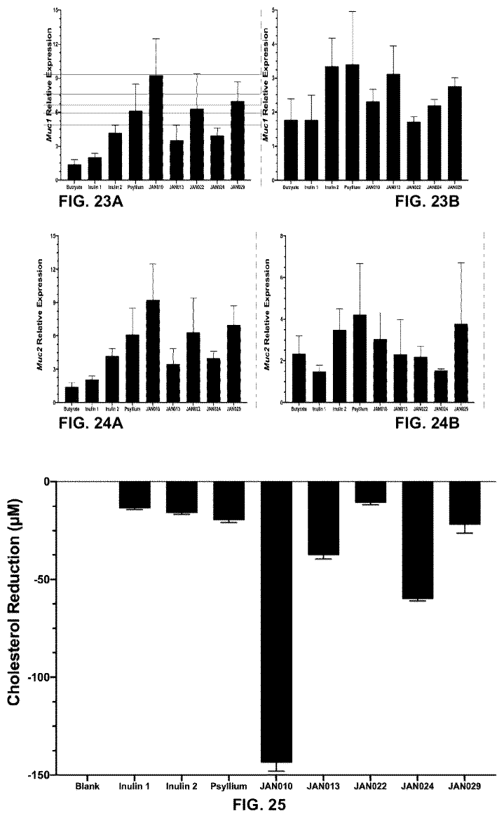

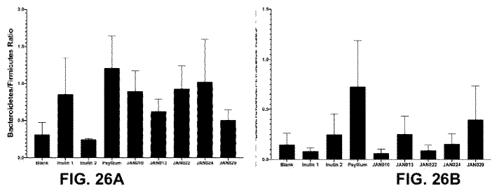

Several fibers were selected for comparison to inulin (two different sources) and Psyllium fiber using the methods described above and healthy or Type II diabetes (T2D) stool samples. FIGS. 13-26 show the results of these assays. FIGS. 13A and 13B show gas production with healthy and T2D samples respectively. FIGS. 14A and 14B show pH with healthy and T2D samples respectively. FIGS. 15A and 15B show total SCFAs with healthy and T2D samples respectively. FIGS. 16A and 16B show GLP-1 secretion with healthy and T2D samples respectively. FIGS. 17A and 17B show PYY secretion with healthy and T2D samples respectively. FIGS. 18A and 18B show IL-10 secretion with healthy and T2D samples respectively. FIGS. 19A and 19B show Gcg expression with healthy and T2D samples respectively. FIGS. 20A and 20B show Pcsk1 expression with healthy and T2D samples respectively. FIGS. 21A and 21B show Pyy expression with healthy and T2D samples respectively. FIGS. 22A and 22B show Tlr4 expression with healthy and T2D samples respectively. FIGS. 23A and 23B show Muc expression with healthy and T2D samples respectively. FIGS. 24A and 24B show Muc2 expression with healthy and T2D samples respectively. FIG. 25 shows cholesterol reduction by the different fibers. FIGS. 26A and 26B show Bacteroidetes/Firmicutes ratio with healthy and T2D samples respectively. FIGS. 13-26 show that several of the fibers tested perform better than inulin or Psyllium fiber.

TABLE 1
Panel of Prebiotic Fibers

| No. | Fiber Type |
|---|---|
| JAN001 | Apple Fiber |
| JAN002 | Pea Fiber |
| JAN003 | Oat Fiber- JRS HF 401 |
| JAN004 | Orange Fiber |
| JAN005 | Inulin- TIC gums |
| JAN006 | Wheat Fiber- JRS |
| JAN007 | Rice Fiber- JRS |
| JAN008 | Gum Arabic |
| JAN009 | Psyllium Husk |
| JAN010 | Locust Bean Gum |
| JAN011 | Guar Gum |
| JAN012 | Kudzu Starch |
| JAN013 | Potato Starch |
| JAN014 | Shitake Mushroom beta glucan |
| JAN015 | Wheat Bran |
| JAN016 | Fibersol SCF |
| JAN017 | High methoxyl Apple Pectin |
| JAN018 | Barley beta glucan |
| JAN019 | Konjac gum |
| JAN020 | Tara gum |
| JAN021 | High Methoxyl Citrus Pectin |
| JAN022 | Sunfiber (partially hydrolyzed guar gum) |
| JAN023 | SWEOAT |
| JAN024 | Concentrated Barley Beta glucan |
| JAN025 | FiberSMART Soluble Corn Fiber |
| JAN026 | Oat bran |
| JAN027 | BGF Immune Beta glucan |
| JAN028 | XOS |
| JAN029 | GOS |
| JAN030 | Barley Bran |
| JAN031 | Chia seed fiber |

TABLE 2
Probiotics

| | | | Exact name of bacterial genus, |
|---|---|---|---|
| | | | species, strain downloaded from |
| No. | Genus | Species | NCBI for analysis |
| 1 | Bifidobacterium | longum** (lactis) | Bifidobacterium longum subsp. infantis |
| | | | ATCC 15697 = JCM 1222 = DSM |
| | | | 20088 |
| 2 | Bifidobacterium | infantis** (lactis) | Bifidobacterium longum subsp. infantis |
| | | | (high GC Gram+) |
| 3 | Bifidobacterium | breve | Bifidobacterium breve DSM 20213 = |
| | | | JCM 1192 |
| 4 | Bifidobacterium | bifidum | Bifidobacterium bifidum ATCC 29521 = |
| | | | JCM 1255 = DSM 20456 (high GC |
| | | | Gram+) |
| 5 | Bifidobacterium | lactis | Bifidobacterium animalis subsp. lactis |
| 6 | Lactobacillus | paracasei | Lactobacillus paracasei |
| 7 | Lactobacillus | plantarum | Lactobacillus plantarum WCFS1 |
| 8 | Lactobacillus | acidophilus | Lactobacillus acidophilus NCFM |
| 9 | Lactobacillus | delbrueckii subsp. | Lactobacillus delbrueckii subsp. |
| | | bulgaricus*(helveticus) | bulgaricus ATCC 11842 = JCM 1002 |
| 10 | Lactobacillus | casei | Lactobacillus casei DSM 20011 = JCM |
| | | | 1134 = ATCC 393 |
| 11 | Lactobacillus | rhamnosus | Lactobacillus rhamnosus GG |
| 12 | Lactobacillus | brevis | Lactobacillus brevis EPS-producing |
| | | | brewery isolates |
| 13 | Lactobacillus | salivarius | Lactobacillus salivarius strain:JCM1046 |
| 14 | Lactobacillus | gasseri | Lactobacillus gasseri ATCC 33323 = |
| | | | JCM 1131 |
| 15 | Lactobacillus | reuteri | Lactobacillus reuteri DSM 20016 |
| 16 | Lactobacillus | helveticus | Lactobacillus helveticus strain: CAUH18 |
| | | | Genome sequencing |
| 17 | Lactobacillus | fermentum | Lactobacillus fermentum IFO 3956 |
| 18 | Lactobacillus | kefiranofaciens | Lactobacillus kefiranofaciens ZW3 |
| | | | genome sequencing |
| 19 | Lactobacillus | kefiri | Lactobacillus kefiri strain:DH5 Genome |
| | | | sequencing |
| 20 | Streptococcus | thermophilus | Streptococcus thermophilus |
| | | | strain: Microbial Genome sequencing |
| 21 | Bacillus | subtilis | Bacillus subtilis, Whole genome |
| | | | sequence of H1 |
| 22 | Bacillus | coagulans | Bacillus coagulans strain: S-lac Genome |
| | | | sequencing and assembly |
| 23 | Bacillus | clausii | Bacillus clausii KSM-K16 |
| 24 | Pediococcus | acidilactici | Pediococcus acidilactici strain:BCC1 |
| | | | Genome sequencing |
| 25 | Lactococcus | lactis biovar diacetylactis | Lactococcus lactis subsp. lactis bv. |
| | | | diacetylactis |
| 26 | Lactococcus | lactis | Lactococcus lactis subsp. lactis I11403 |
| 27 | Lactococcus | cremoris | Lactococcus lactis subsp. cremoris MG1363 |
| 28 | Leuconostoc | dextranicum | Leuconostoc mesenteroides subsp. |
| | | | dextranicum strain:DSM 20484 Genome |
| | | | sequencing |

TABLE 3

| No. | Bioactive |
|---|---|
| Bio1 | Turmeric |
| Bio2 | Resveratrol |
| Bio3 | Curcugen |
| Bio4 | Turmeric Extract Powder, |
| Bio5 | Oligonol (Lychee fruit |
| | extract) |
| Bio6 | Green Tea extract |
| Bio7 | Green Tea extract |
| Bio8 | Actazin (Yellow kiwi fruit |
| | extract) |
| Bio9 | Livaux (Green kiwi fruit |
| | extract) |
| Bio10 | Benifuuki green tea |
| | powder |
| Bio11 | Fenufibers (fenugreek seed |
| | extract) |
| Bio12 | Silbinol ( [symbol] |
| | [symbol] |

Effectiveness of JAN1000 as a Dietary Supplement

The following experiments the role of the product JAN1000 with greatly increasing the production of total short-chain fatty acids (SCFA) and, in particular, the short-chain fatty acid butyrate. SCFAs play key roles as modulators of health. For example, SCFAs may reduce risks of inflammatory diseases, Type II diabetes, obesity, heart disease, and other conditions, as well as promote colon health.

The experiments include in vitro benchmarking studies testing effectiveness of JAN000 against gold-standard supplements for increasing production of SCFAs, namely inulin and Psyllium husk. The studies show that JAN1000 compares favorably to at least these two products with respect to production of SCFAs, and for providing beneficial immune, hormonal, and metagenomic effects. Additionally, the experiments provide evidence that JAN1000 improves glucose, immune, and lipid homeostasis. The experiments also provide evidence that JAN1000 is an anti-hyperlipidemic, promotes satiety, increases populations of known beneficial bacteria associated with good health and health outcomes and reduces populations of those associated with poor health and health outcomes, is an anti-hyperglycemic, an anti-inflammatory, and improves epithelial barrier function. Additionally, JAN1000 may include prebiotic fibers, probiotics, and polyphenols.

JAN1000's constituent ingredients have calorie counts consistent with the FDA's National Drug Code (NDC) guidelines.

| Major carbohydrate and calorie | | |
|---|---|---|
| contributing ingredients | Carbohydrates | Calories |
| Resistant Potato Starch | 5.515 | 22.032 |
| Partially Hydrolyzed Guar Gum | 4.890 | 9.906 |
| Oat β-Glucan | 2.232 | 10.26 |
| Locust Bean Gum | 2.88 | 6.156 |
| Barley β-Glucan | 2.19 | 6.39 |
| Probiotics | 0.1754 | 0.97 |
| Polyphenols | 1.1213 | 5.297 |
| Total | 19.0037 | 61.011 |
| | (Rounded to 19) | (Rounded to 60) |

Particular experiments compare the metabolic benefits of JAN1000 to currently-marketed standard fiber products (e.g., Psyllium husk and inulin). Generally, the experiments use six healthy and six Type-II diabetes donors' stool samples. The samples were fermented in triplicate.

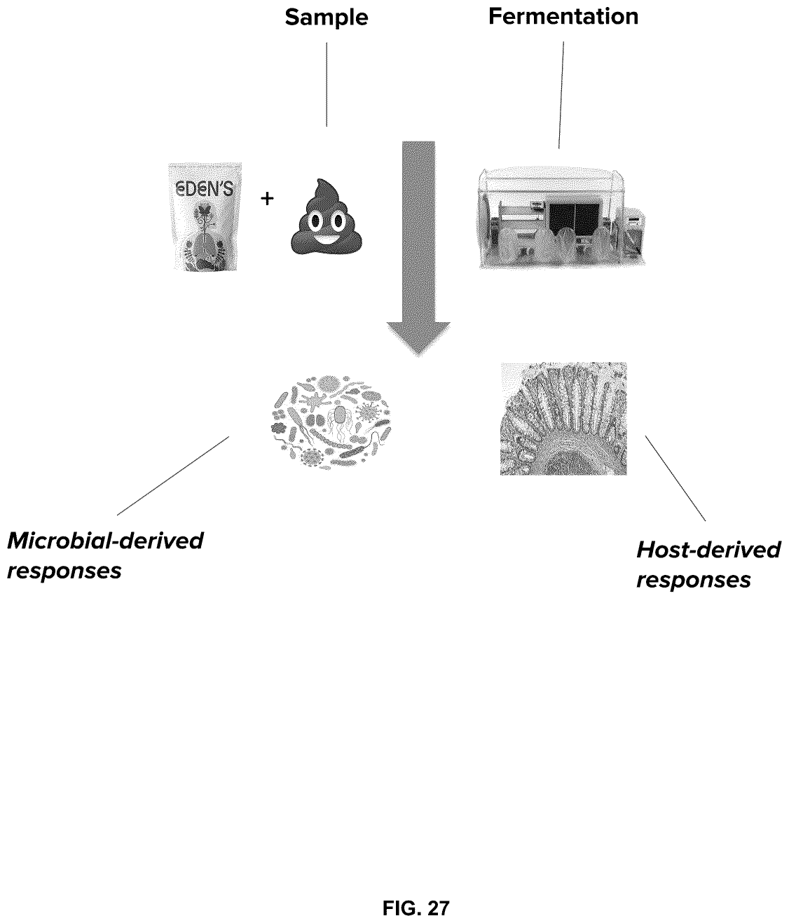

FIG. 27 illustrate general conditions of the disclosed experiments. Generally, human subjects are treated with JAN1000, Psyllium husk, and inulin. The experiments take stool samples following treatment, which are then fermented. Data is collected from the fermented samples.

Assays are performed in triplicate, with equivalent doses across treatment groups. Dose-response curves are calculated for some experiments. For some experiments, monosaccharide compositional analysis is performed by high-performance anion-exchange chromatography with pulsed amperometric detection (HPAEC-PAD). For some experiments, gas chromatography-mass spectrometry (GC-MS) validation may be performed orthogonally. Post fermentation samples are measured by semi-quantitative thin layer chromatography (TLC) and quantitative gas chromatography-flame ionization detection (GC-FID), orthogonal. Orthogonal measurement between TLC and GC-FID may be appropriate because compared to TLC, GC-FID is more sensitive but lower throughput.

Microbiome abundance and functional profiling are measured via shallow shotgun metagenomics. Metabolic and immune hormones & cytokines are also measured. The experiments include measurement of hormone & cytokine gene expression using a reverse transcriptase-quantitative polymerase chain reaction (RT-qPCR) TaqMan® assay. These experiments, including orthogonal measurement of RT-qPCR and ELISA, serve to increase confidence in experimental results and confirm that hormones, peptides, and cytokines are associated with relevant expressions of key genes and pathways.

The experiments resulted in microbial-derived responses captured from human intestinal microbiome, such as those from serotonin, GABA, spermidine, arachidonic acid, choline, glutathione, folic acid, short-chain fatty acids, medium-chain fatty acids, branched-chain fatty acids, and indoles. The experiments captured host-derived responses from primary human colonocytes include metabolic responses such as glucagon-like peptide 1 (GLP-1), peptide tyrosine tyrosine (PYY), glucagon gene (Gcg), and proprotein convertase subtilsin/kexin (Pcsk1). Immune responses such as IL-10, Tlr4. The experiments also captured responses associated with gut permeability such as those from mucin 1 (Muc1) and mucin 2 (Muc2).

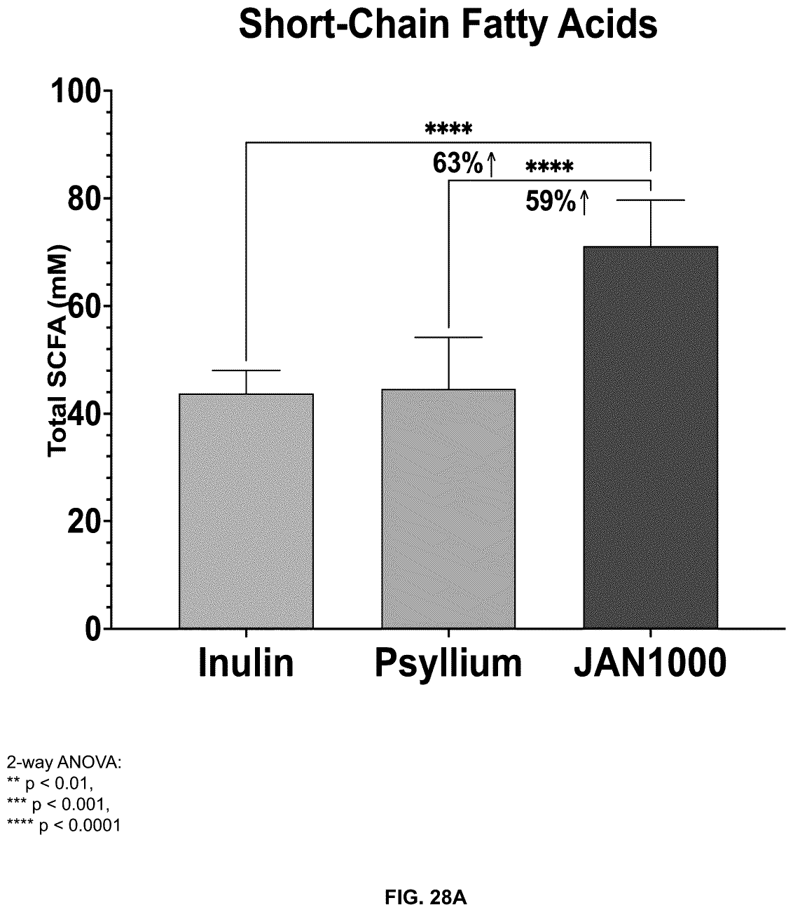

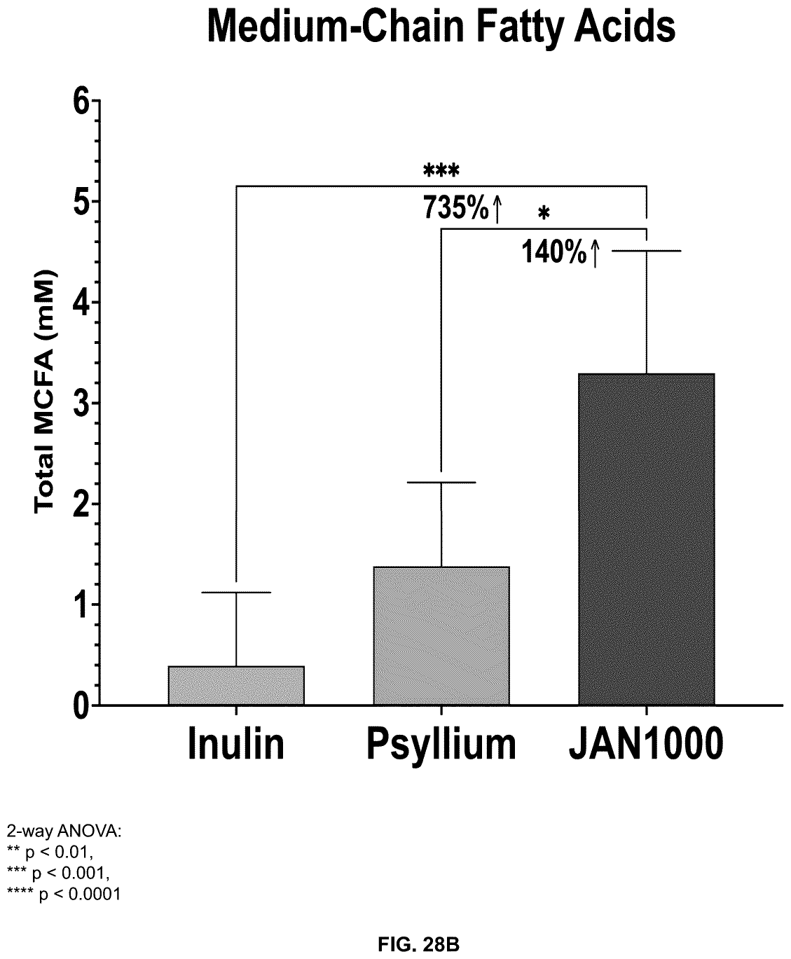

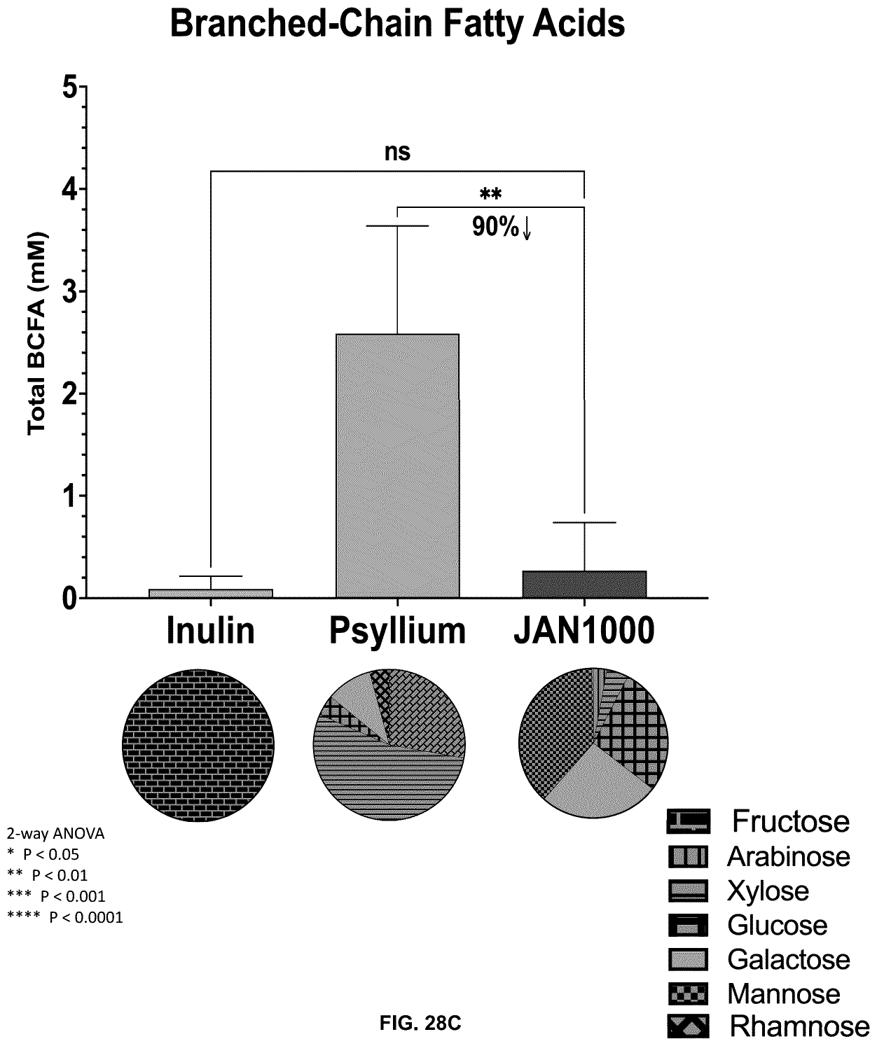

FIGS. 28A-28C illustrate the results of experiments that compare production of fatty acids produced by inulin, Psyllium, and JAN1000. The experiments show that use of the product JAN1000 selectively increases production of SCFA and medium-chain fatty acids (MCFA).

As a rich source of dietary fiber, JAN1000 is consumed by clades of bacteria and selectively augments the production of beneficial short- (SCFA) and medium-chain fatty acids (MCFA) to limit the production of branched-chain fatty acids (BCFA).

A combination of SCFA and MCFA can augment hormonal response through activation of free fatty acid receptors 2 and 3 (FFAR2/3) (SCFA) and FFAR1/3/4 (MCFA) downstream signaling pathways. Even though the concentration of SCFAs is ten times greater than MCFAs in the intestines, MCFAs are potent activators of FFAR1, FFAR3, and FFAR4. BCFAs have been associated with hypercholesterolemia and dyslipidemia in people with metabolic syndromes.

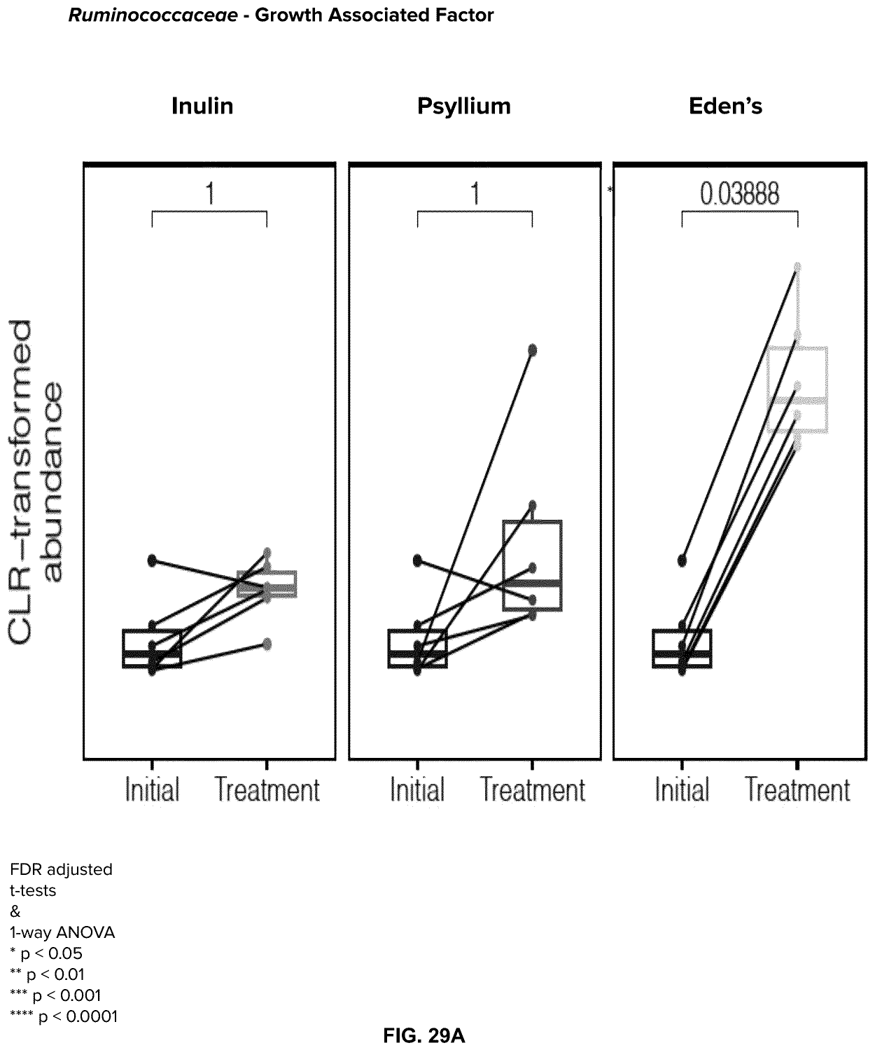

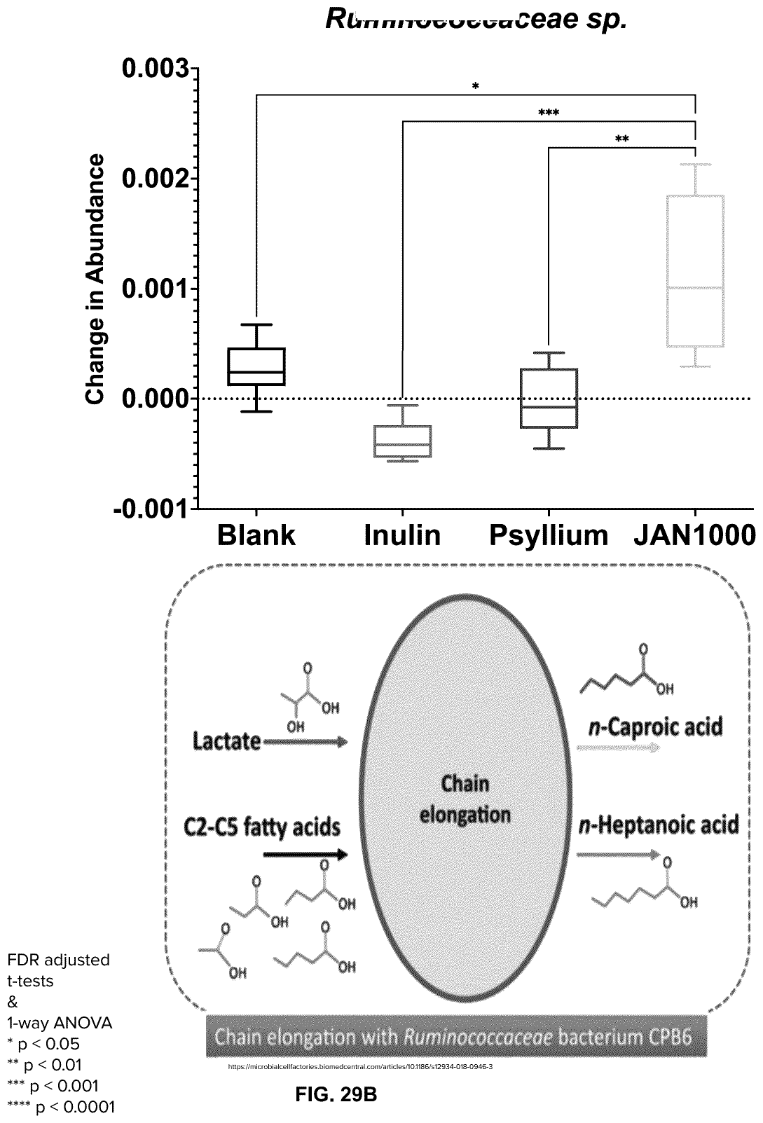

FIGS. 29A-29B illustrate experiments showing JAN1000's effect on a chain elongation process. The results show that JAN1000 supports the growth of a bacteria that supercharges SCFA to MCFA. While the majority of MCFAs come from dietary triglycerides, some bacteria store energy in the form of MCFA converted from SCFA or lactate. A benefit of the energy-rich MCFAs produced by JAN1000 is that the MCFAs augment the benefits of SCFA to gut health. JAN1000 has significantly targeted a species of Ruminococcaceae that serves this chain elongation niche.

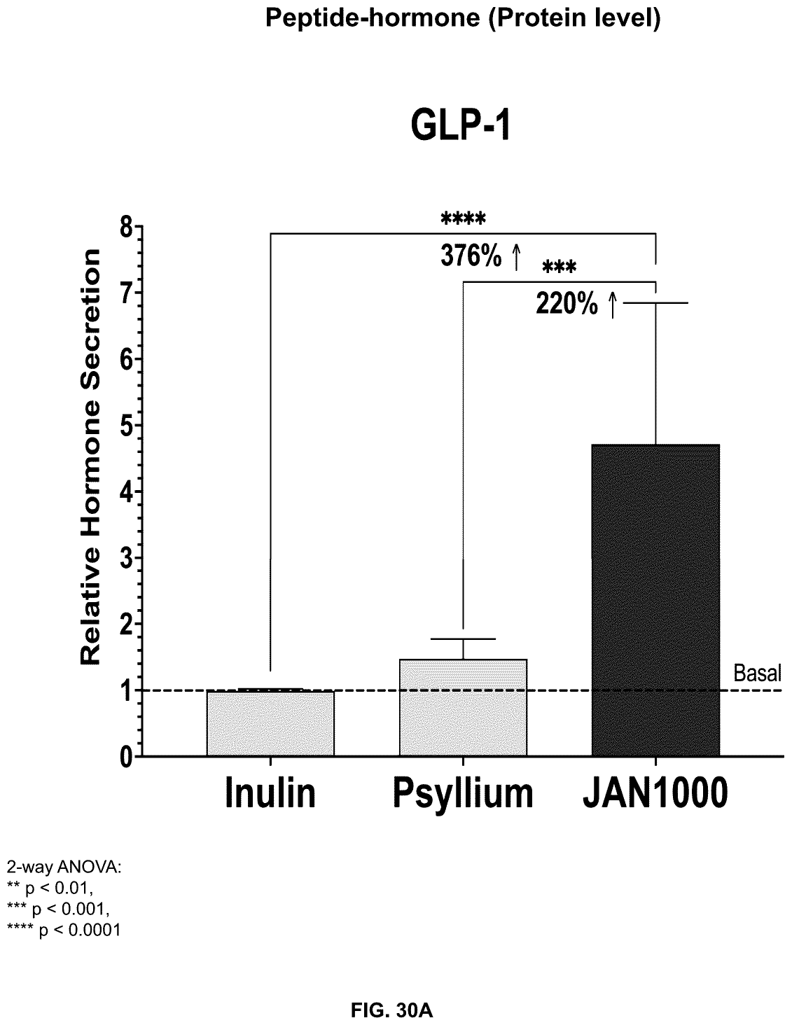

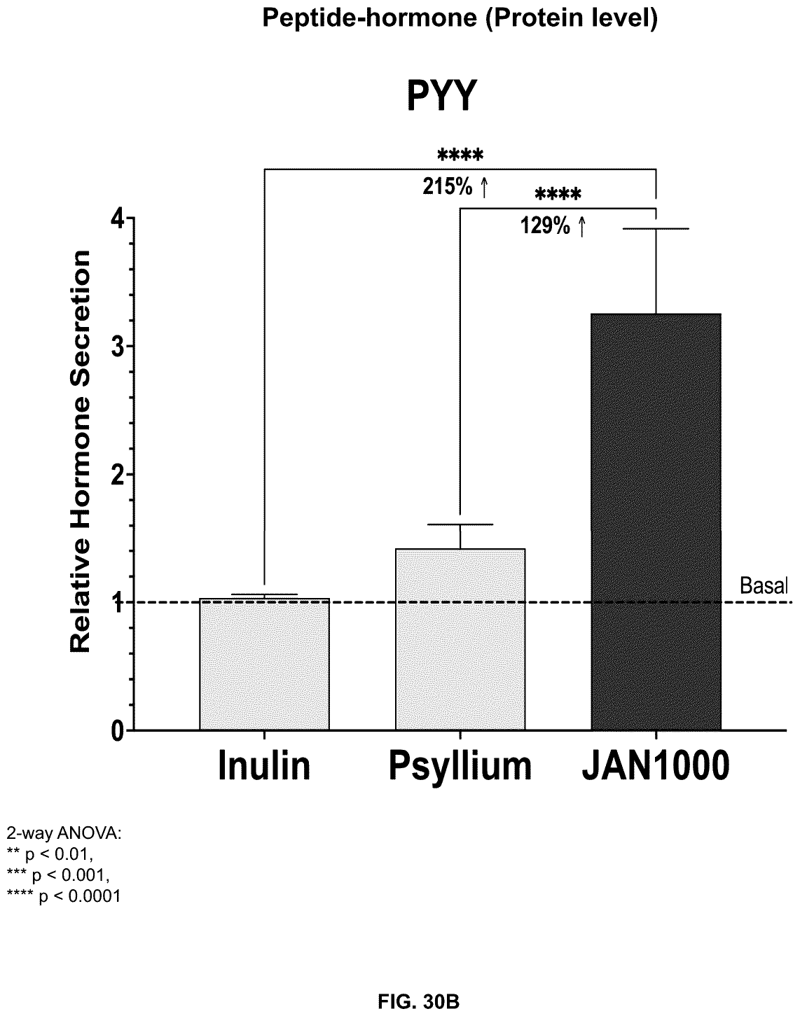

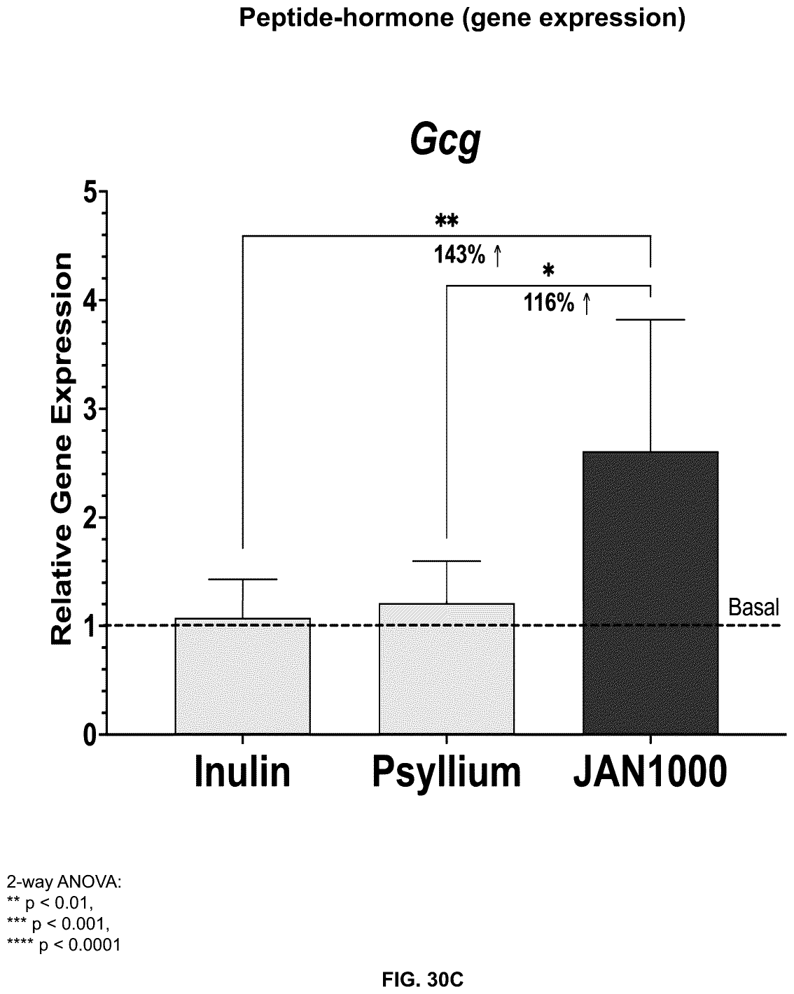

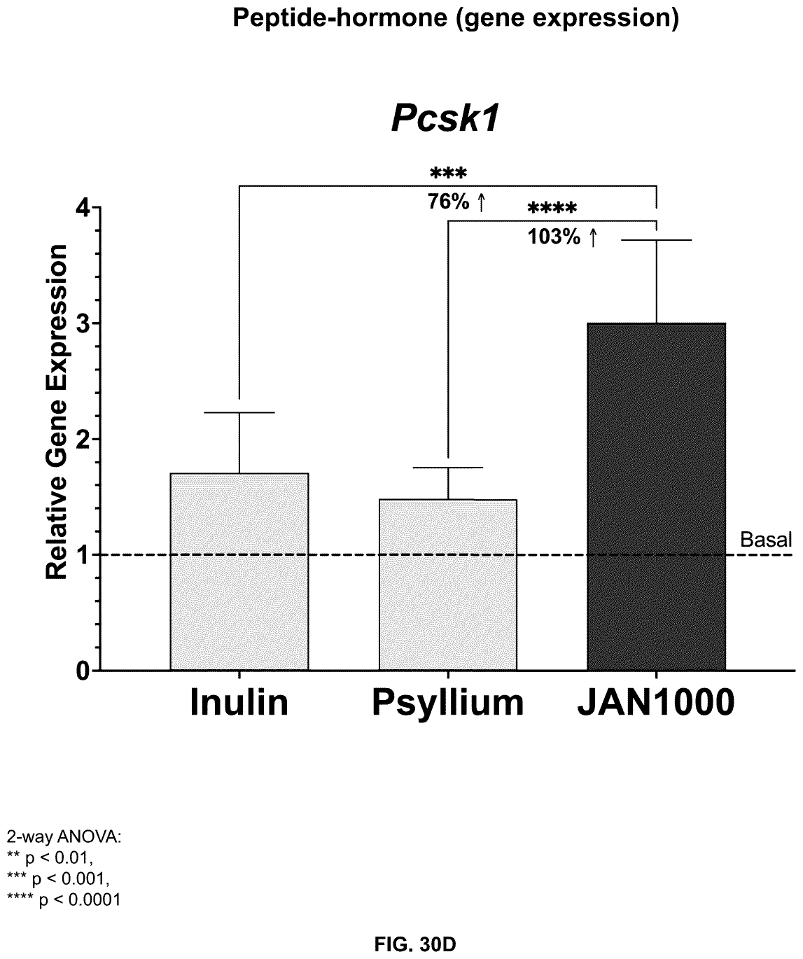

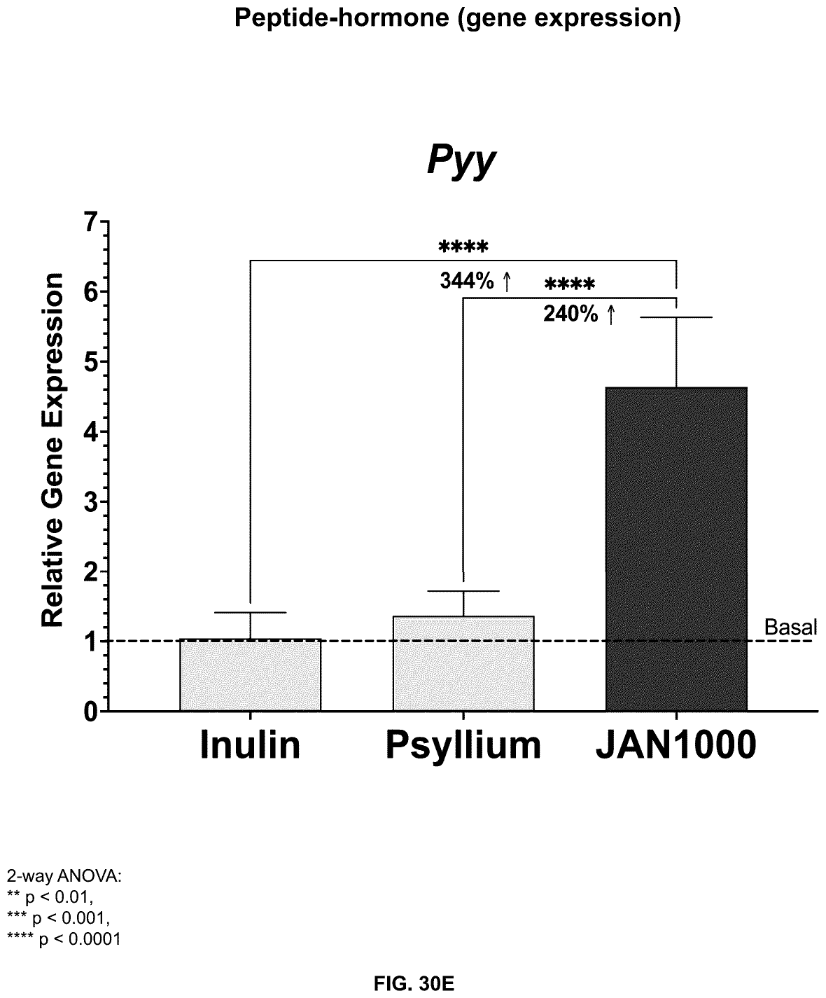

FIGS. 30A-30E illustrate the comparative effect of JAN1000 on regulators of metabolic health. The results show that JAN1000 is effective with respect to augmenting regulators of metabolic health. The plots illustrate that, compared to inulin and Psyllium, JAN1000 increases relative hormone secretion of GLP-1 and PYY. Additionally, JAN1000 increases gene expression compared to inulin and Psyllium of Gcg and Pcsk1.

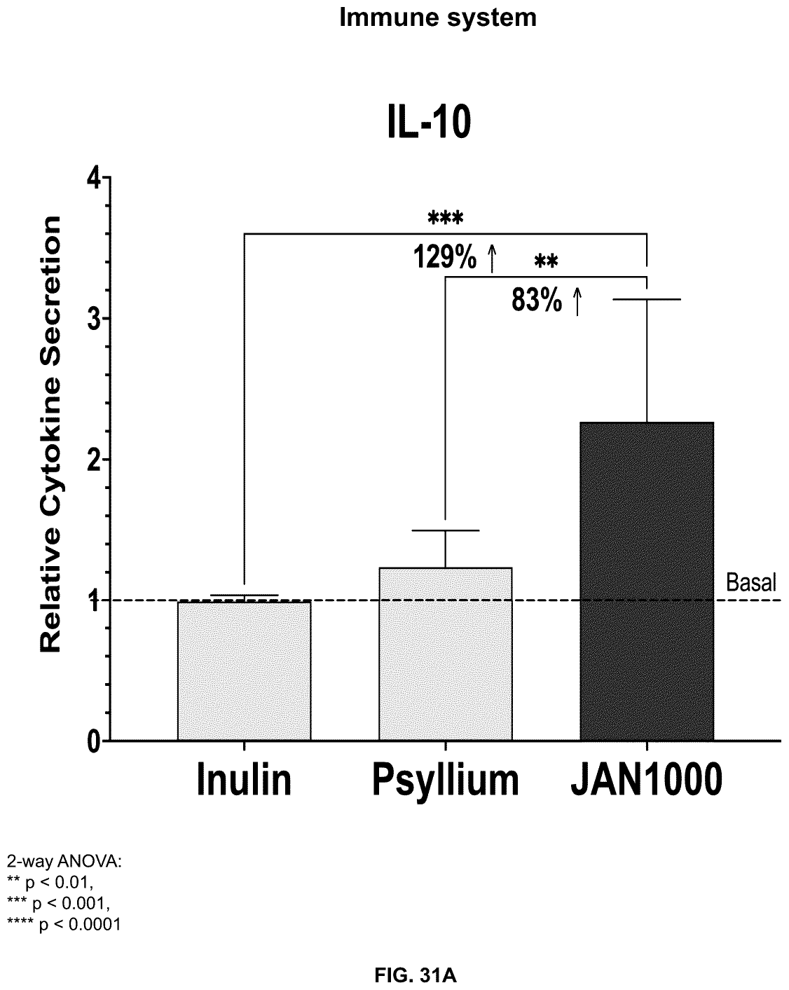

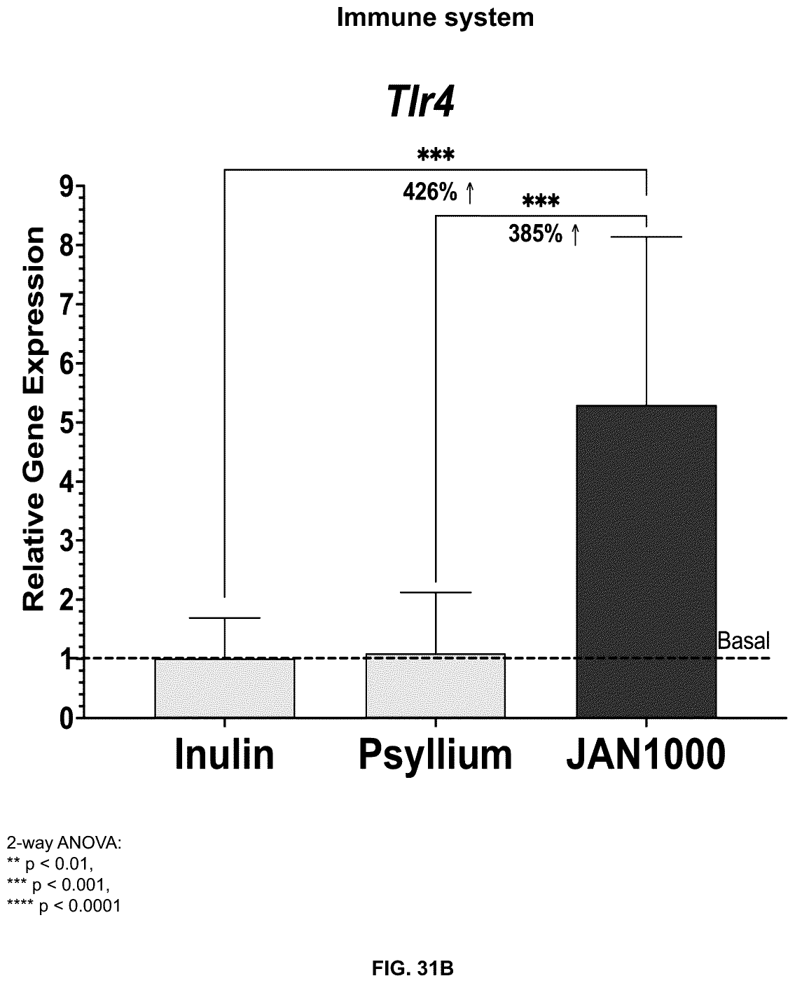

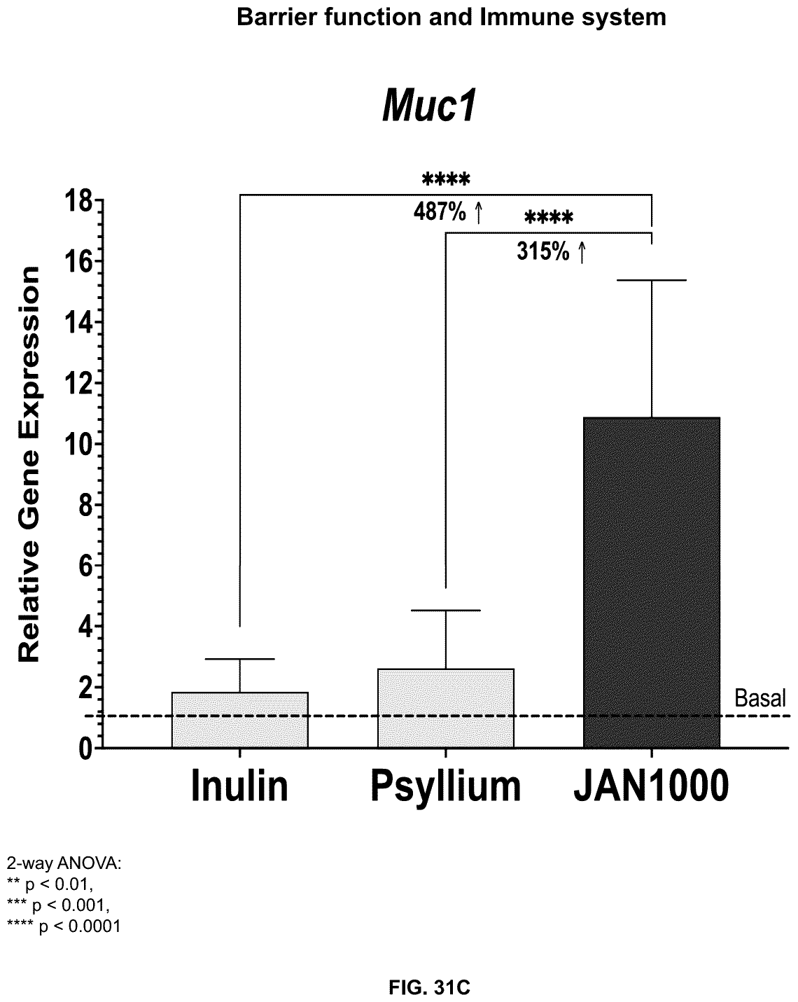

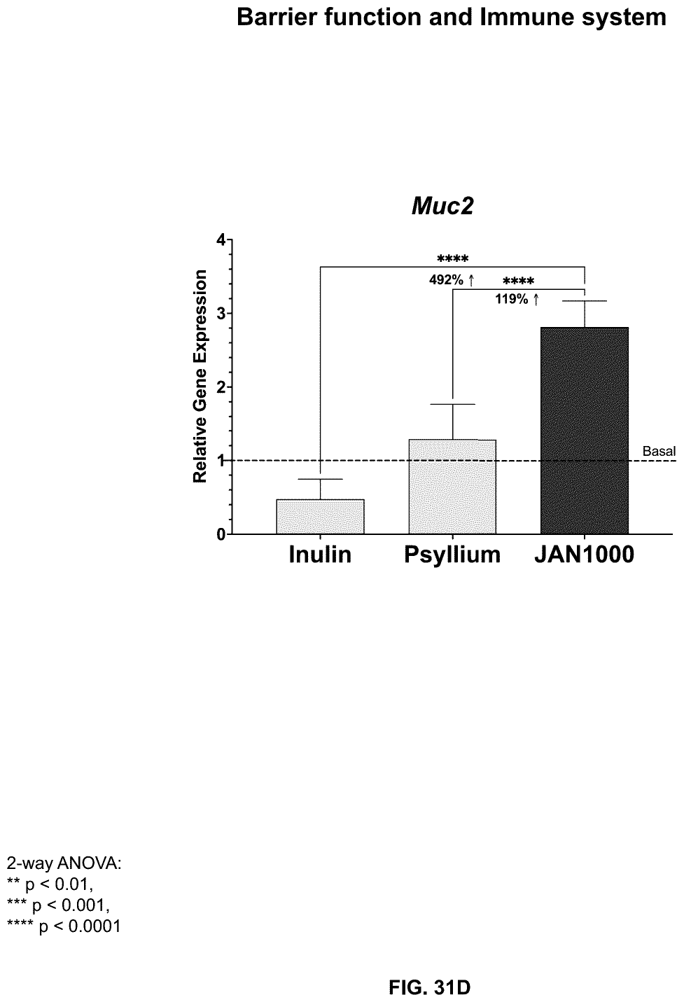

FIGS. 31A-31D illustrate the comparative effects of JAN1000 to inulin and Psyllium on regulators of intestinal immune system and mucosal integrity. The results show that JAN1000 effectively activates regulators of intestinal immune system and mucosal integrity. While Psyllium may slightly induce an interleukin-10 (IL-10) signal above basal, only JAN1000 is able to induce Tlr4. This is significant, because IL-10 produced in enterocytes is generally localized, while Tlr4 induction can lead to regulation of a cascade of cytokines, chemokines and chemotactic factors.

Thus, JAN1000 is more effective at improving immune tone via the gut locally with minimal systemic immune modulation. Additionally, JAN1000 is also a potent activator of mucin pathways involved in regulation of tight-junctions, gut permeability, and host immune defense systems. These findings are consistent with the roles of SCFA, indoles and gamma-aminobutyric acid (GABA) production by JAN1000.

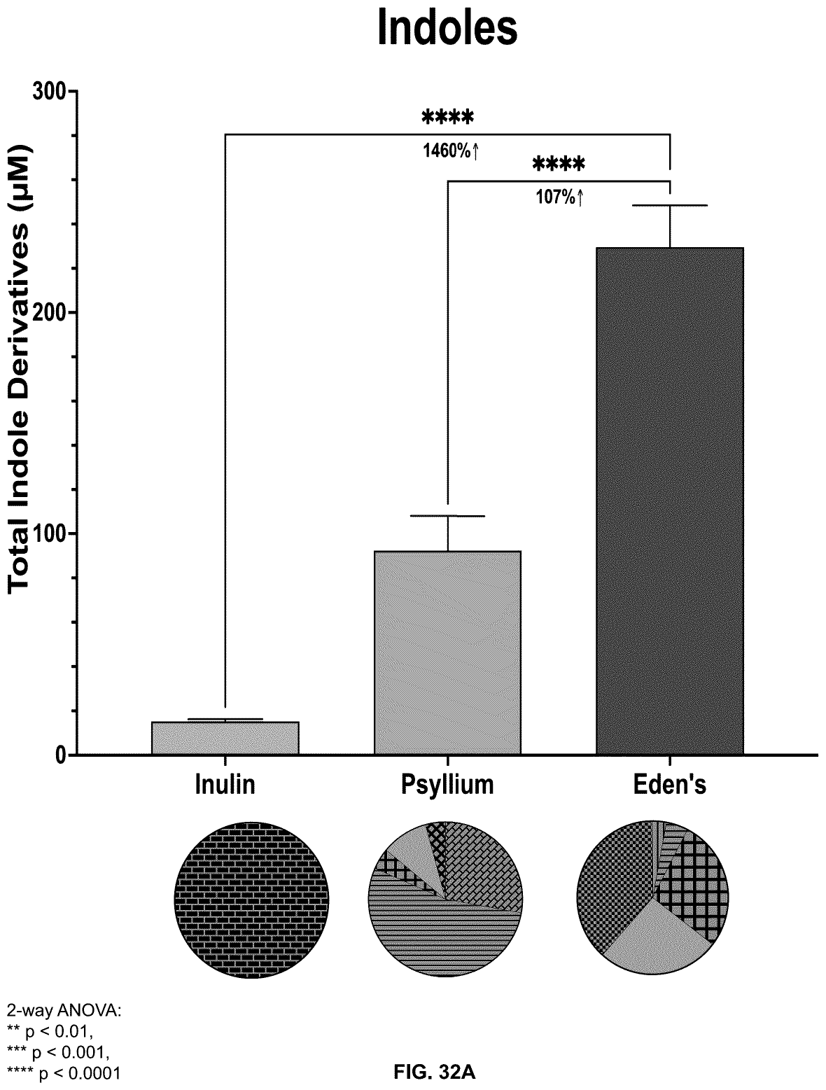

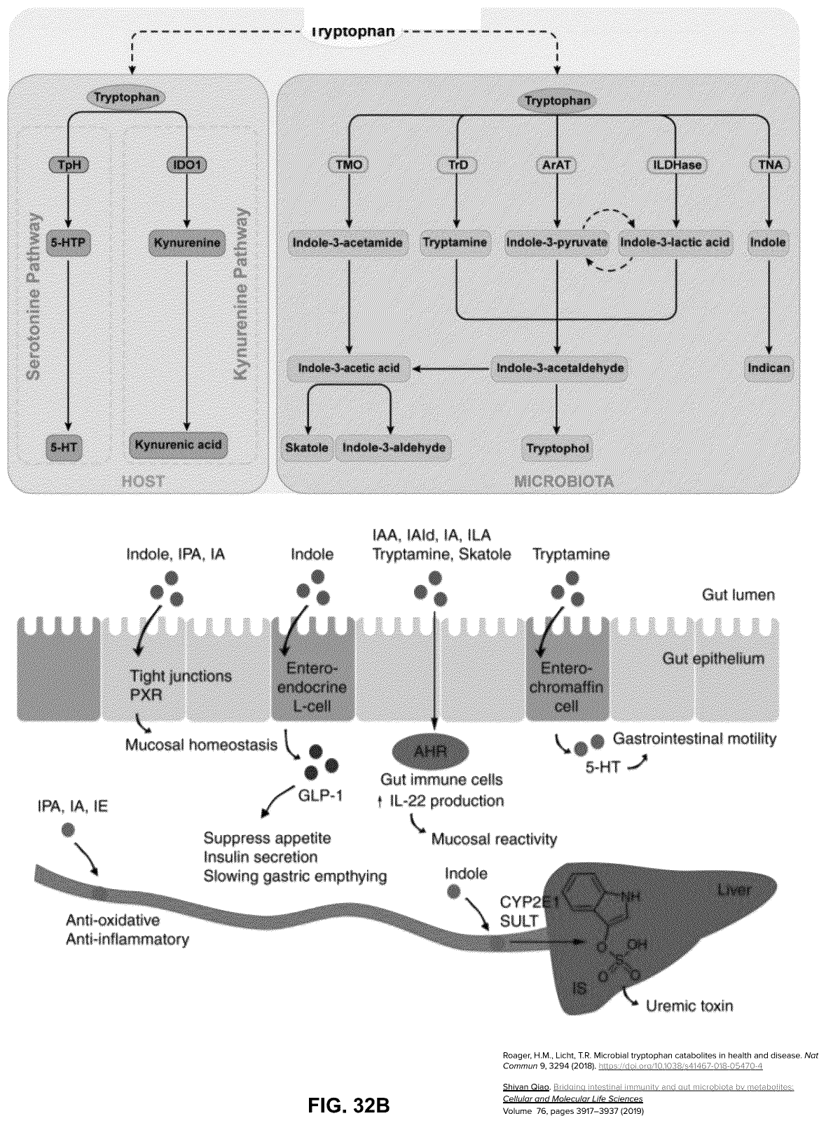

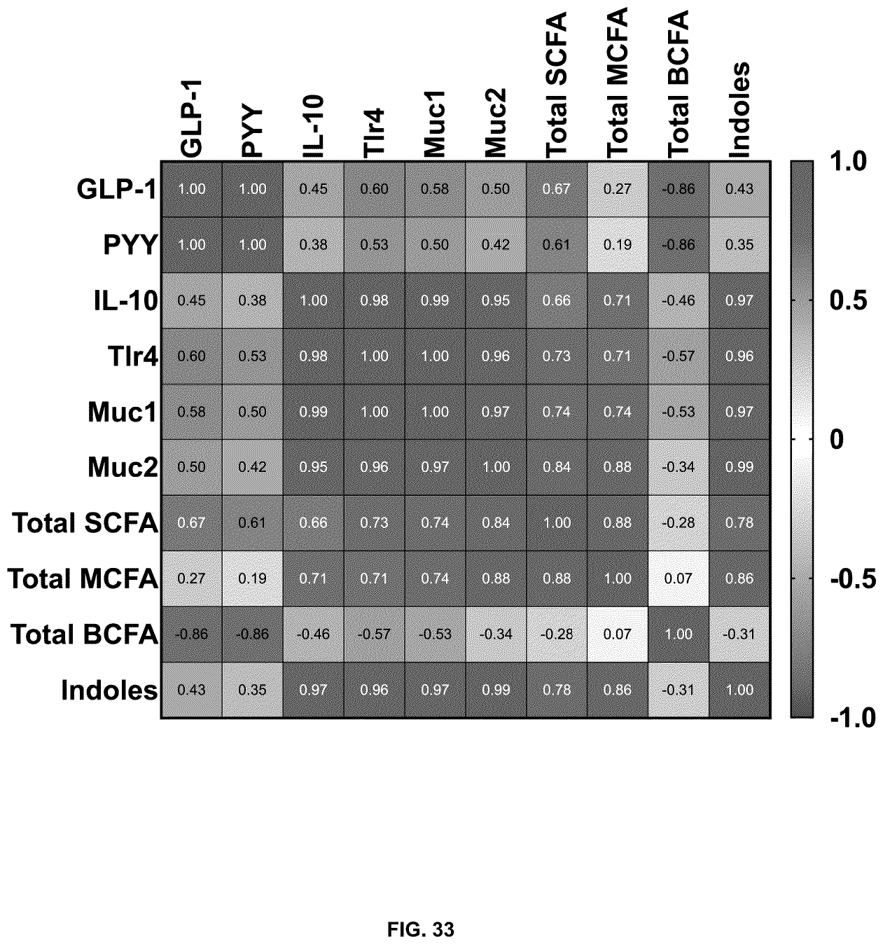

FIGS. 32A-32B illustrate results showing that JAN1000 not only promotes carbohydrate fermentation, but it has the ability to boost microbial metabolism of other macromolecules. As major bacterial tryptophan metabolites, indoles are recognized signaling molecules involved in the regulation of intestinal, metabolic, and immune health. JAN1000 promotes indole development by converting tryptophans, brought in by JAN1000's plant-based ingredients as well as endogenous tryptophan from stools, into signaling molecules that maintain gut health. Tryptophan serves as a versatile precursor molecule, which can be converted to a diverse set of indoles by specific gut bacteria which yield unique functionality. Studies suggest that there is an etiological relationship between tryptophan and its derivatives and gut inflammation. Indole research is a deep and wide space with evidence in anticancer, antiviral, antimicrobial, anti-inflammatory, anti-HIV and antidiabetic activities.

FIG. 33 shows a plot that illustrates associations of SCFA and MCFA productions with host-secreted hormones and cytokines. SCFA and MCFA production mediated by JAN1000 is positively correlated with host-secreted hormones and cytokines. Total SCFA are strongly associated with all three peptide hormones and cytokine of interest (GLP-1, PYY, and IL-10). MCFA are strongly associated with immune biomarkers: IL-10, Tlr4, Muc1, and Muc2. Indoles are also strongly correlated with the immune biomarkers. BCFA are negatively correlated with GLP-1, PYY, IL-10, Tlr4, Muc1, and Muc2. SCFA and BCFA are inversely correlated. Enteroendocrine cells (EEC) of the intestine express multiple nutrient sensing receptors, particularly for SCFA and MCFAs: probable G-protein coupled receptor 84 (GPR84) and FFARs 1-4. Increased gut MCFA levels via JAN1000 can engage EEC and the enteric nervous system resulting in positive metabolic effects.

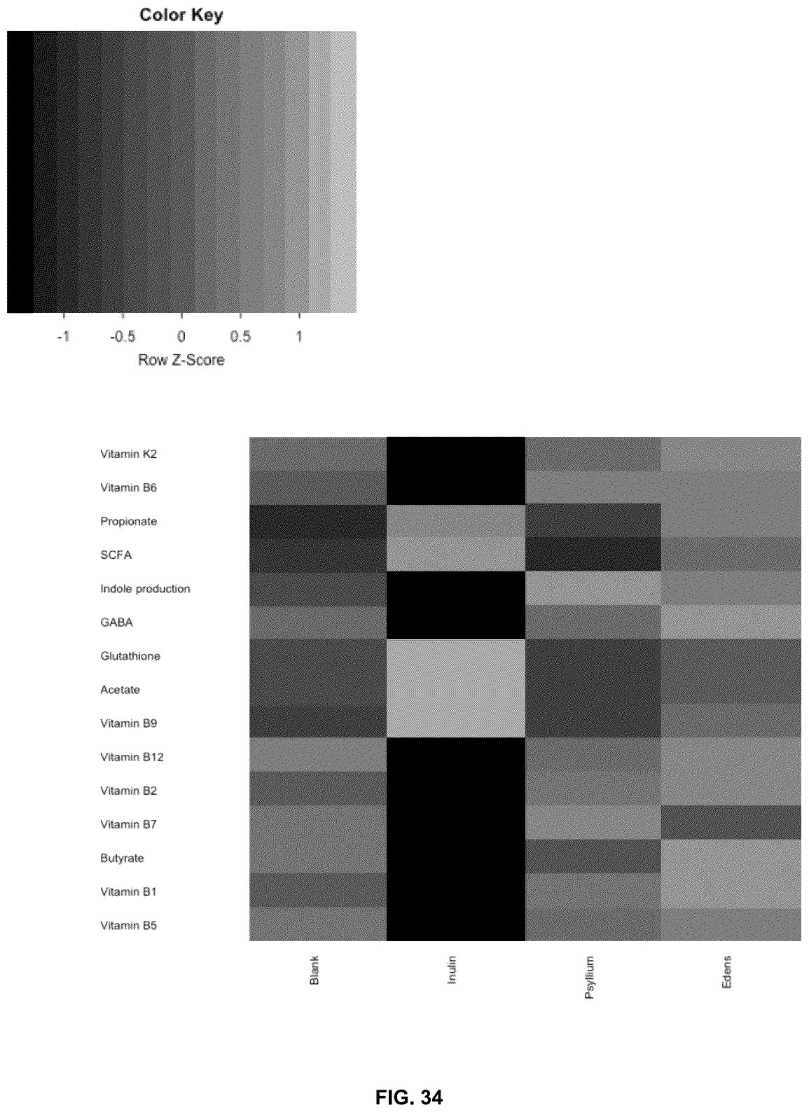

FIG. 34 illustrates a plot that shows JAN1000's effects on the production of health promoting neurotransmitters and metabolites. The plot illustrates that JAN1000's metagenomic functional profile supports the production of health-promoting neurotransmitters and metabolites. Clear trends in SCFA production are evident in the genetic capability of the microbiome. JAN1000 increases the functional potential of butyrogenic pathways. JAN1000 robustly increases the functional potential to produce Gamma-aminobutyric acid (GABA), which helps control feelings of fear & anxiety, depression, inflammation, and pain. Inulin is the most polarizing dietary fiber source since it is a simple carbohydrate source. As a simple carbohydrate, inulin FOS does not require vitamin cofactors for metabolism, whereas mixed carbohydrate sources of Psyllium and JAN1000 yield greater genetic potential for vitamin biosynthesis. Vitamin B and K biosynthetic pathways are associated with regulation of host immunity.

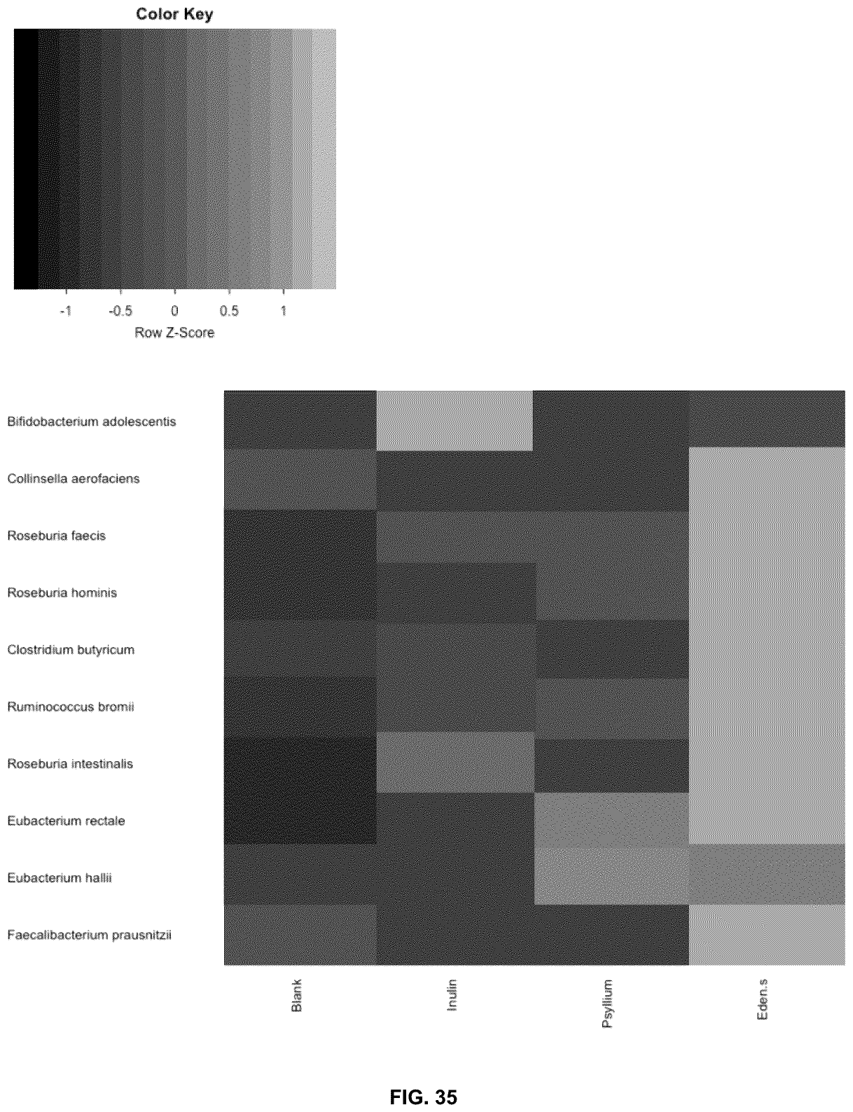

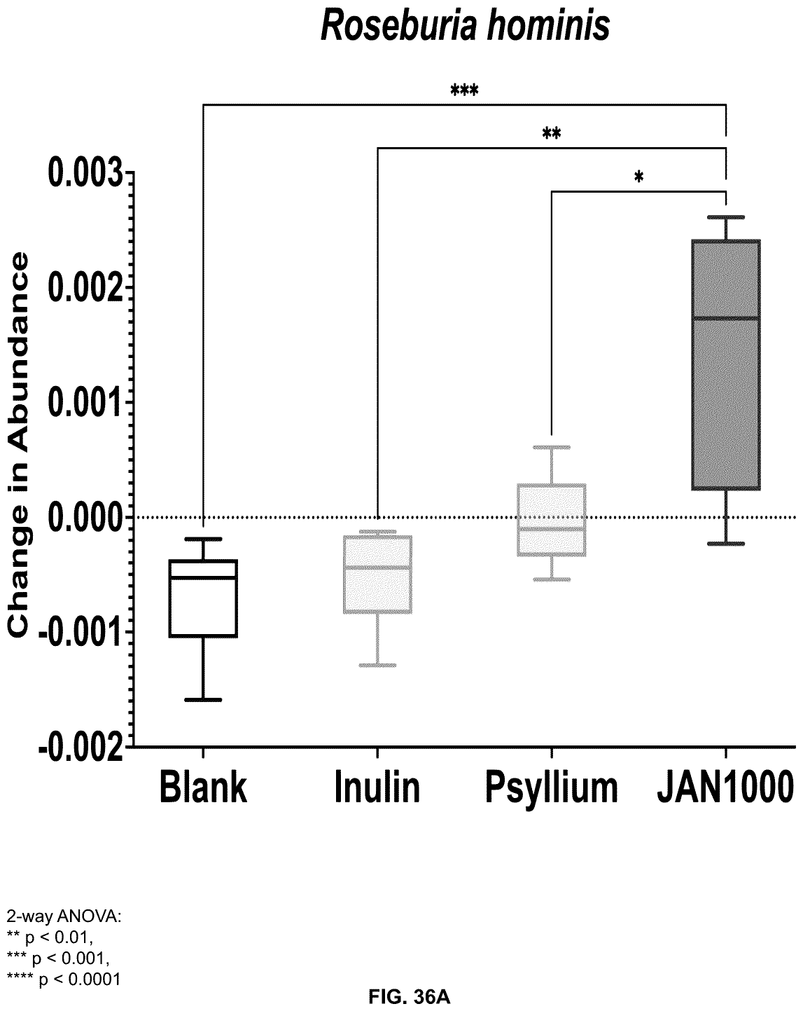

FIG. 35 illustrates a plot that shows JAN1000's effects on promoting growth of various types of bacteria. The plot shows that JAN1000 robustly fuels the growth of a consortium of keystone bacteria. Inulin specifically feeds Bifidobacterium adolescentis, which supports the acetate and lactate production pathways. Psyllium specifically targets Eubacterium hallii which is a butyrogenic species. In addition to targeting these bacteria, JAN1000 also targets Eubacterium hallii and a close relative, Eubacterium rectale, both strong butyrogenic species. These keystone species have been previously demonstrated to interact synergistically to break down fiber into beneficial microbial metabolites. These specific bacteria have been associated with metabolic disease recovery across a number of studies.

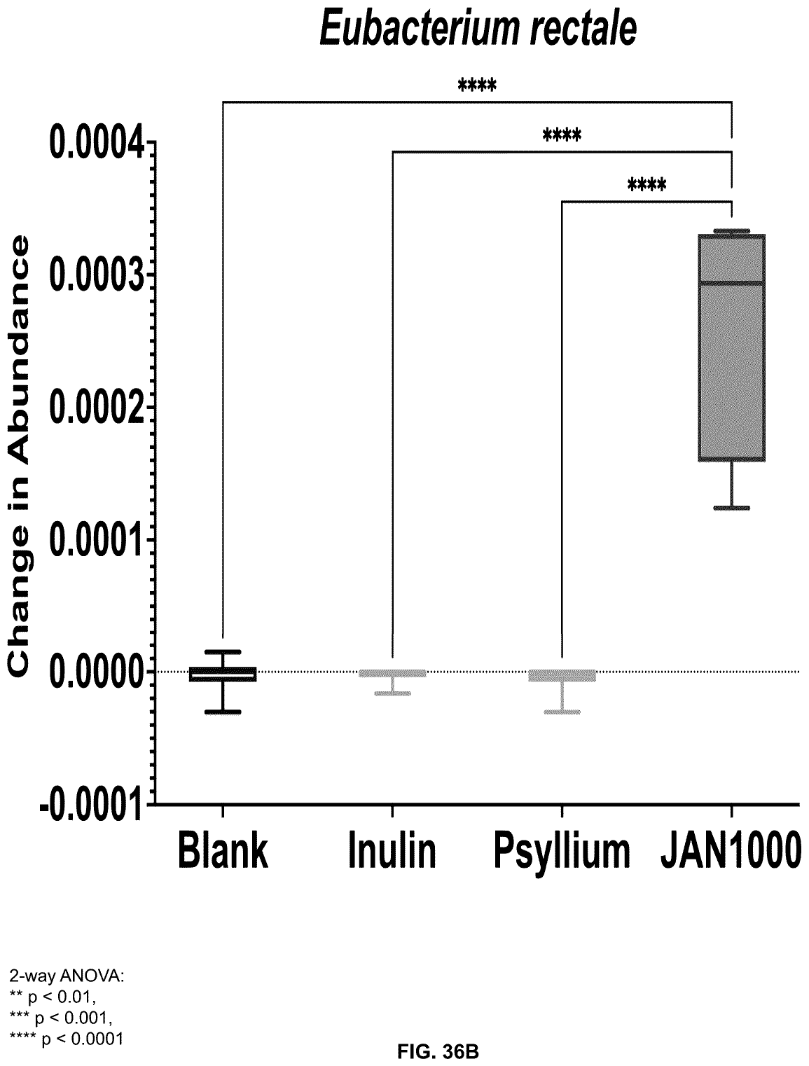

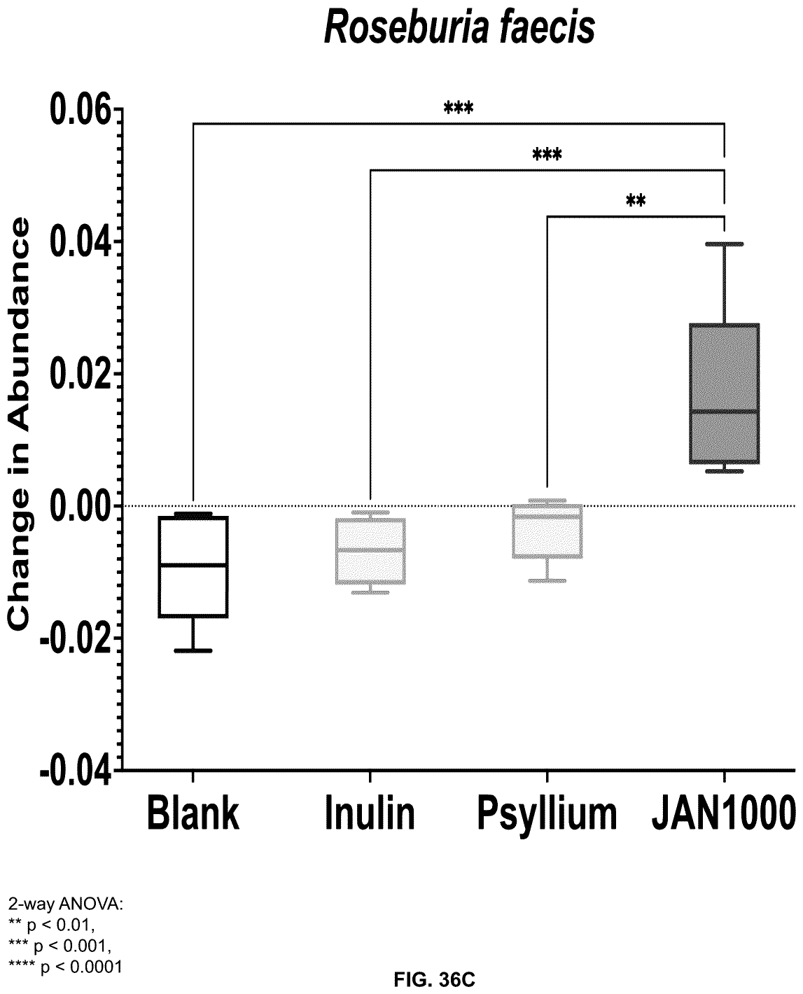

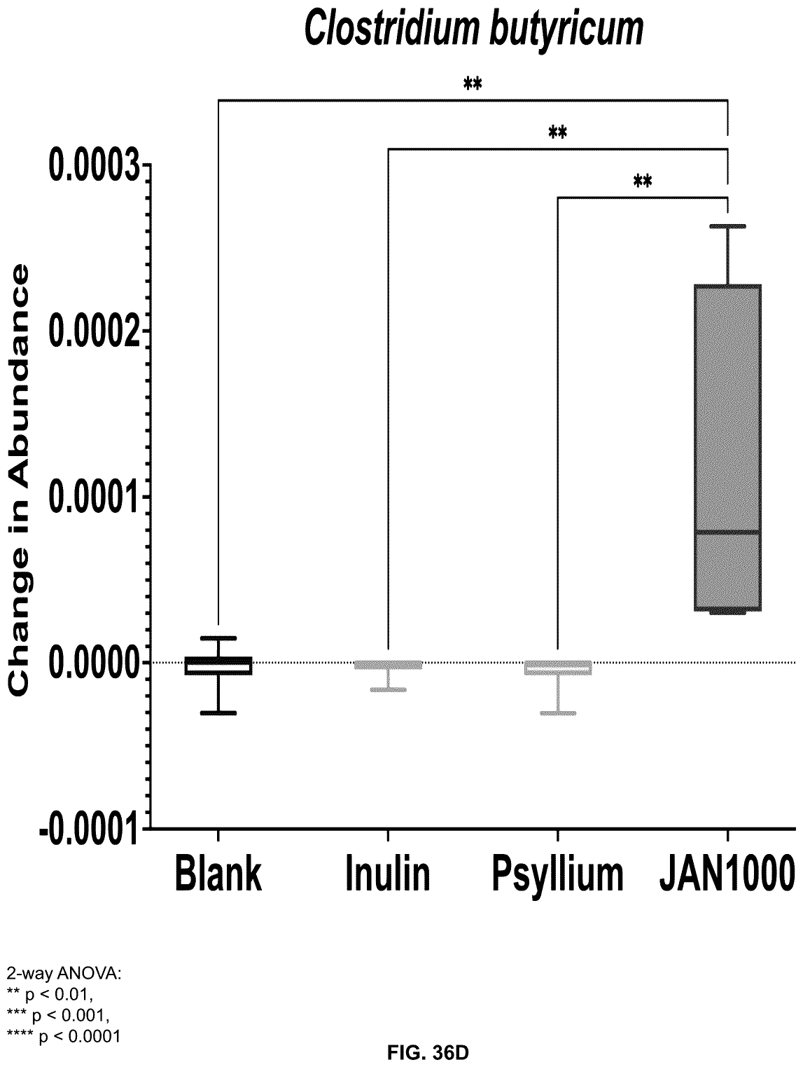

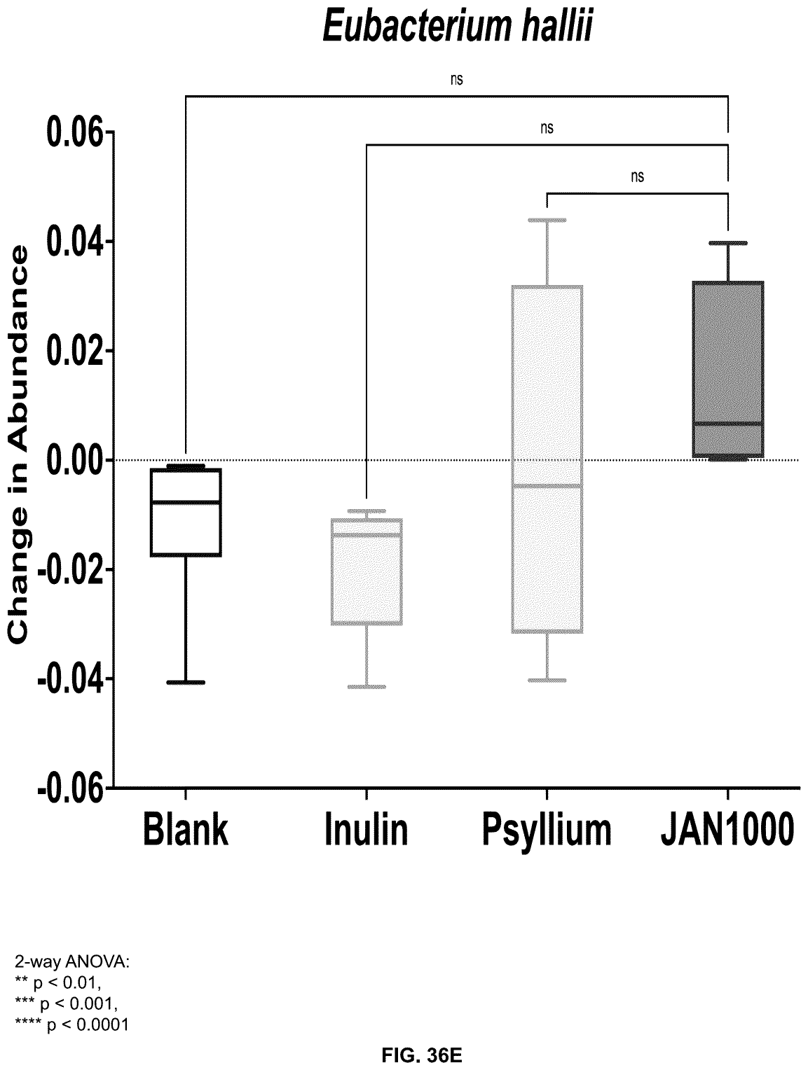

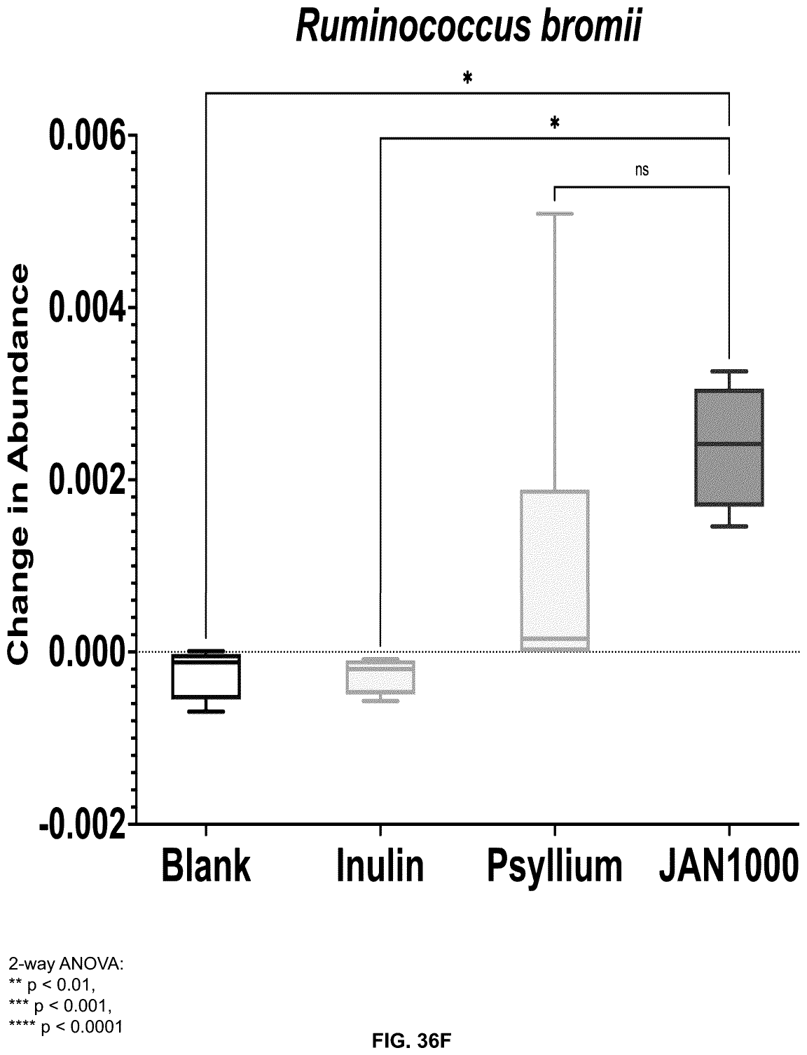

FIGS. 36A-36F illustrate the capability of JAN1000 to grow existing low populations of butyrogenic species. In addition to F. prausnitzii and C. aerofaciens, JAN1000 promotes growth of a host of butyrogenic keystone species. By contrast, inulin and Psyllium are not effective modulators of keystone butyrogenic bacteria.

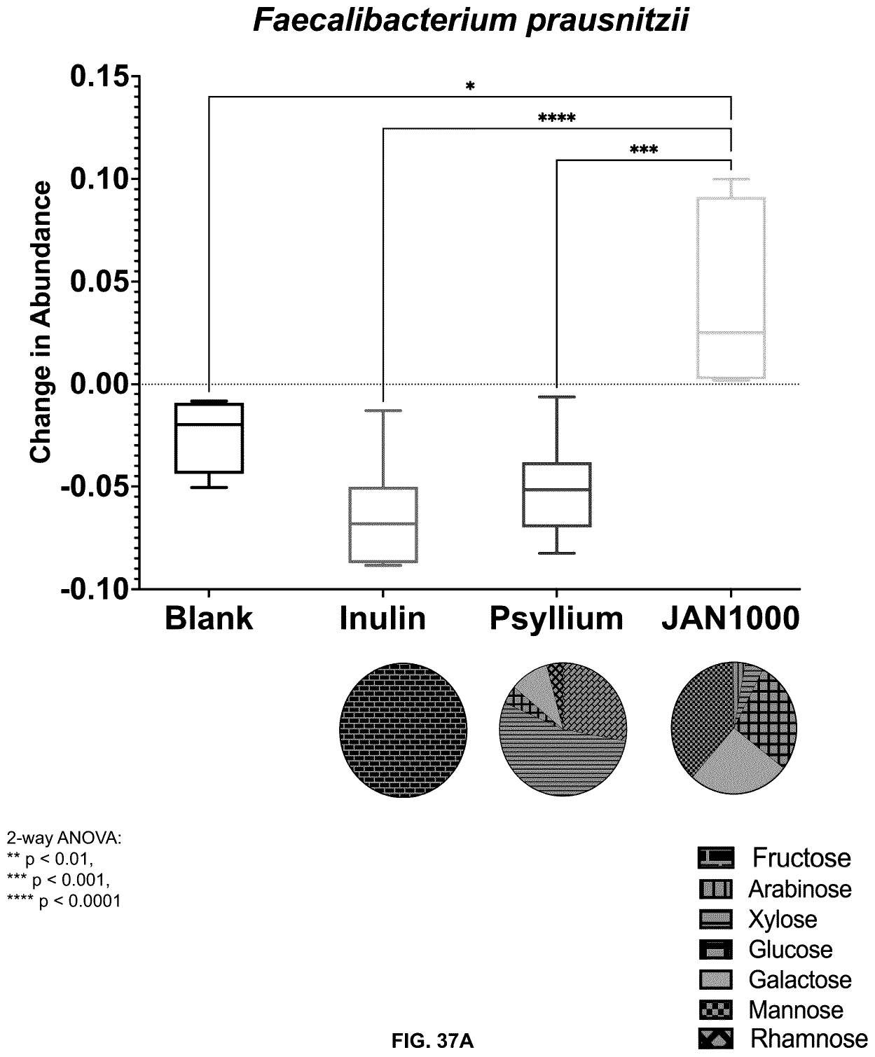

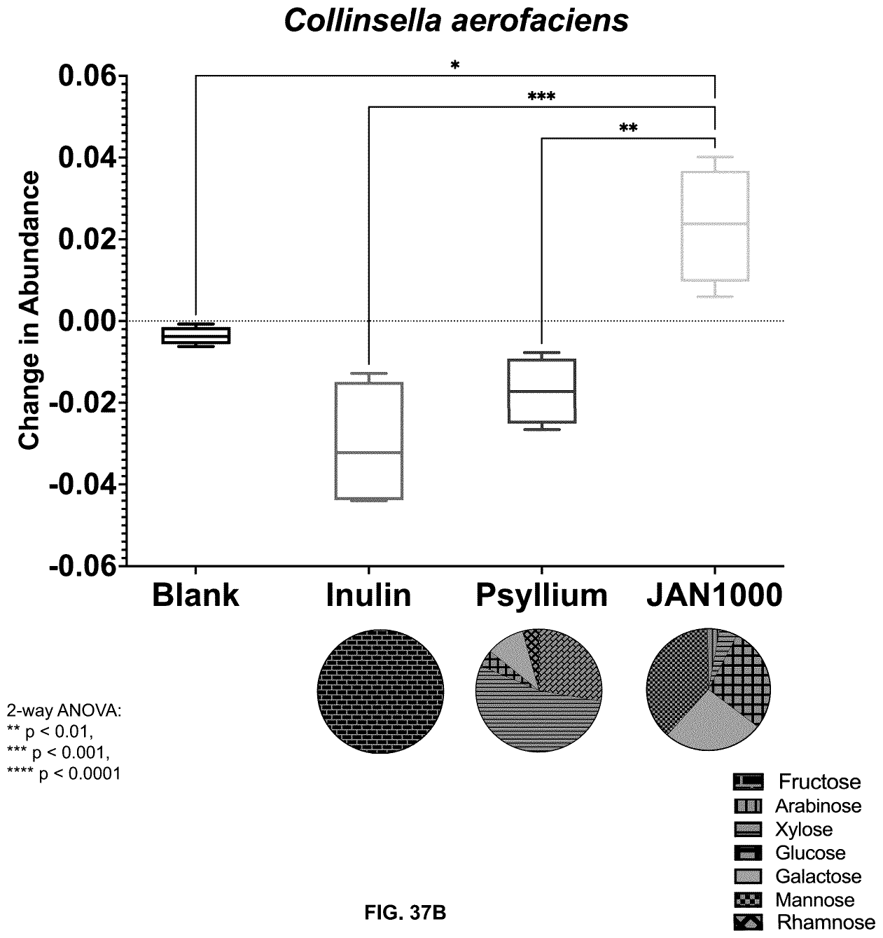

FIGS. 37A-37B illustrates a plot that shows JAN1000's effectiveness as a carbon source for particular bacteria when compared to inulin and Psyllium. JAN1000 is an effective carbon source for two keystone species associated with T2D amelioration. F. prausnitzii is a bioindicator of human health with anti-inflammatory and metabolic effects. F. prausnitzii has shown to grow butyrogenically on β-mannose oligosaccharides. JAN1000 is a strong source of β-galactomannan and serves to feed this correctional keystone species. Similarly, Collinsella aerofaciens ferments mannose, glucose, and galactose in butyrate over arabinose and xylose. JAN1000 provides dietary glucose via resistant starch and β-glucans, which feed C. aerofaciens, while the Psyllium supplement provides primarily arabinose and xylose.

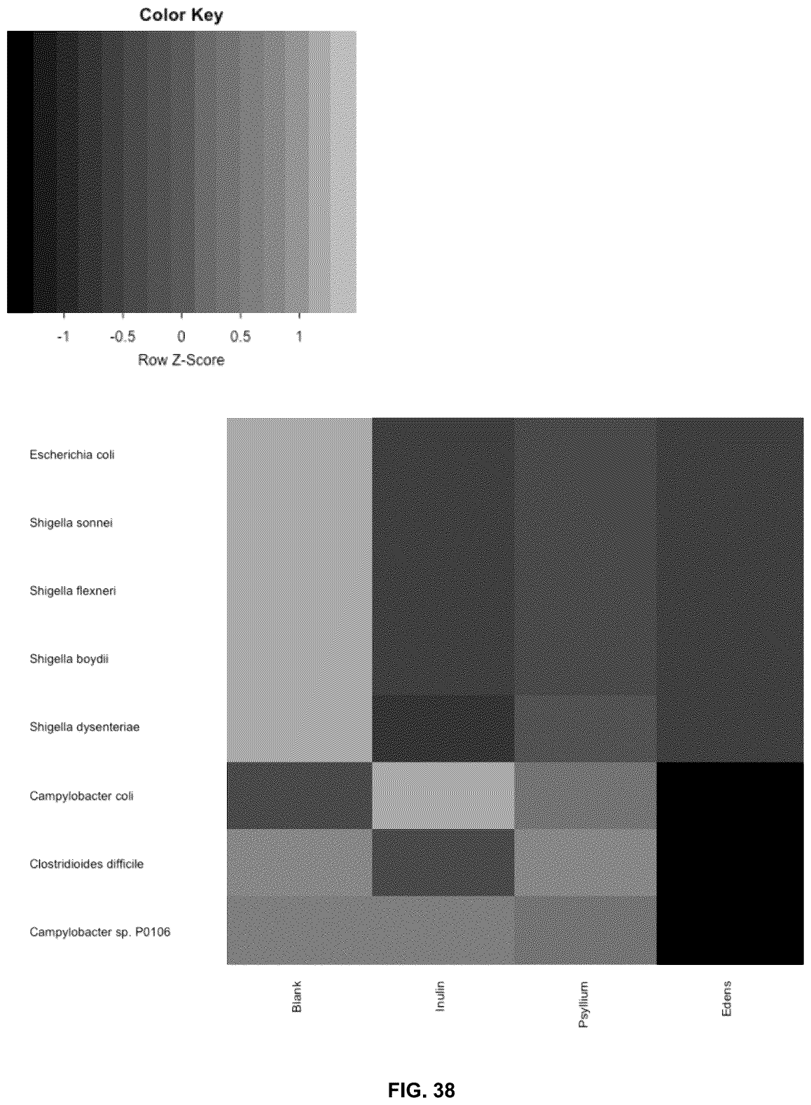

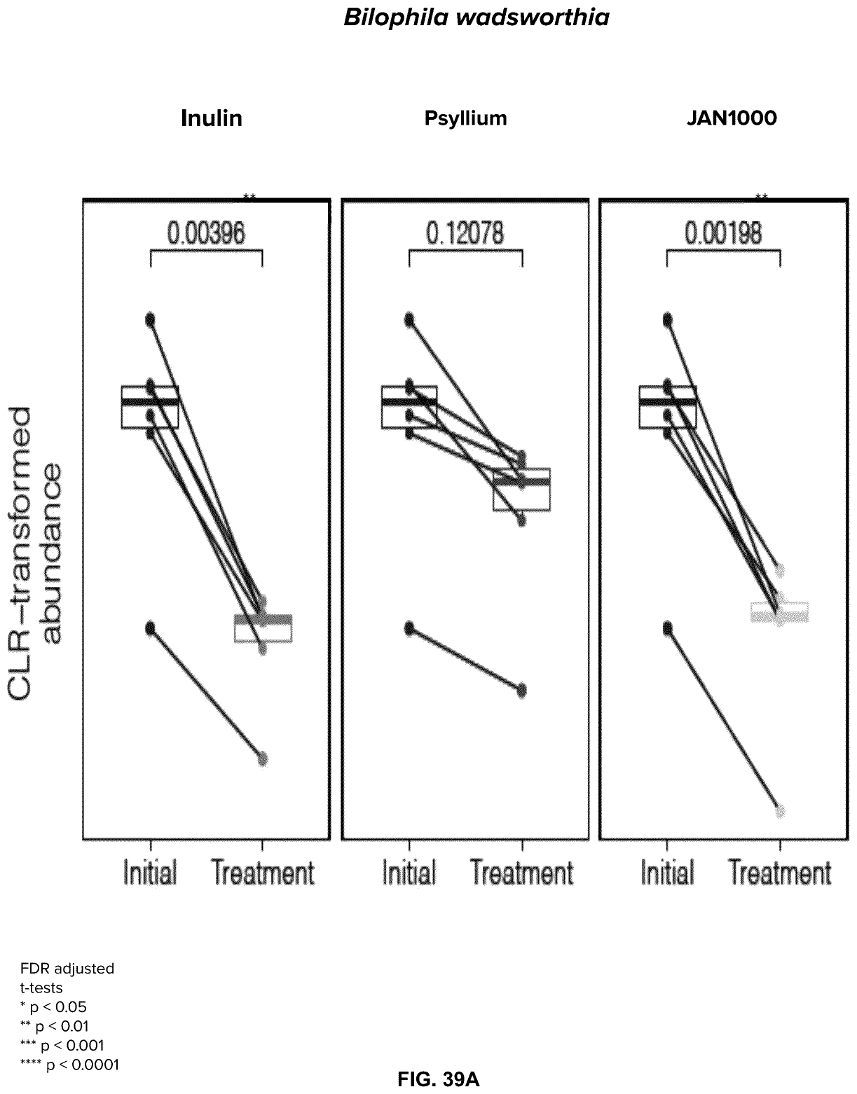

FIG. 38 illustrates a plot that shows JAN1000's effect on opportunistic pathogens. The plot shows that JAN1000 prevents the growth of opportunistic pathogens. All prebiotic supplements appear to lower the growth of phenotypically related: E. coli and Shigella. These populations follow similar abundance patterns, but these species are distinguishable via metagenomics. JAN1000 hinders the growth of pathogens evident in all stool donors. Specifically, JAN1000 significantly inhibits growth of Campylobacter and Clostridioides difficile. In contrast, both inulin and Psyllium induce Campylobacter. Additionally, Psyllium causes an increase in Clostridioides difficile populations.

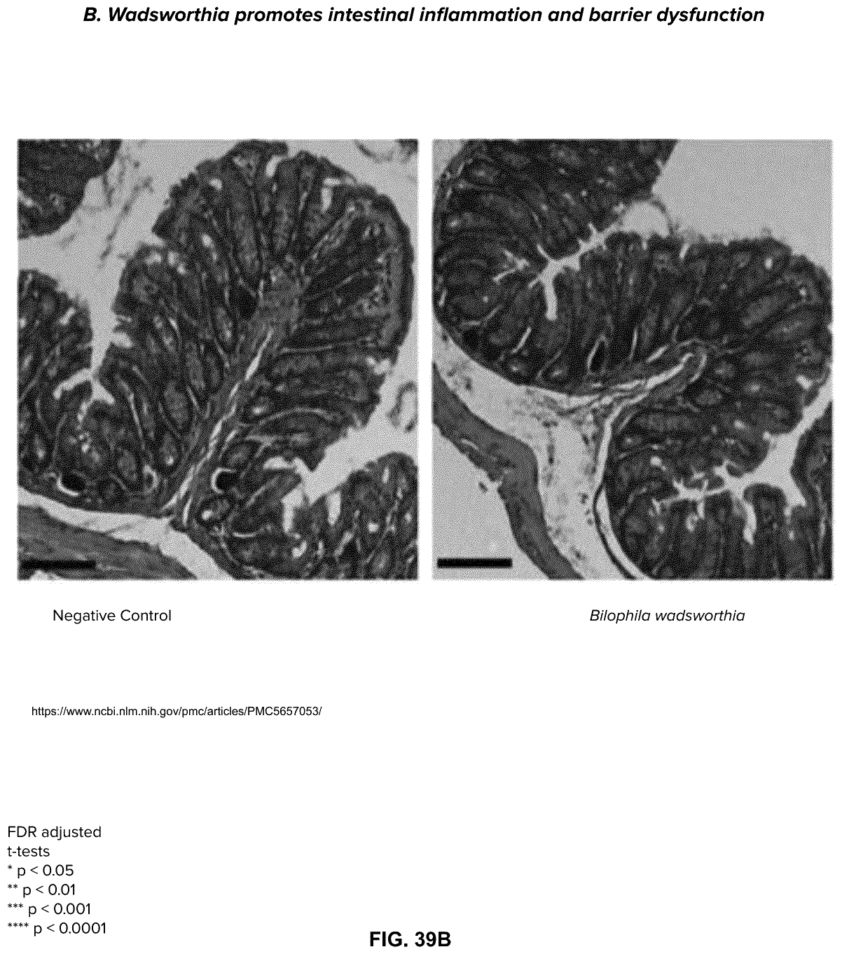

FIGS. 39A-39B illustrates experimental results showing how JAN1000's effect on Bilophila wadsworthia. The plots show that JAN1000 promotes intestinal and metabolic health by suppressing the growth of Bilophila wadsworthia. Bilophila wadsworthia is a hydrogen sulfide-causing strain that is associated with gut inflammation and increased intestinal permeability (also referred to as “leaky gut”). Compromised gut barrier function is associated with pathogenesis of intestinal and metabolic disorders. B. wadsworthia has been both associated with and shown to aggravate disease states caused by high-fat diet. While inulin and Psyllium also suppressed B. wadsworthia, JAN1000 provided the most significant and consistent suppression. Lowering hydrogen sulfide levels by reducing the population of B. Wadsworthia can be an effective strategy for maintaining optimal intestinal barrier function.

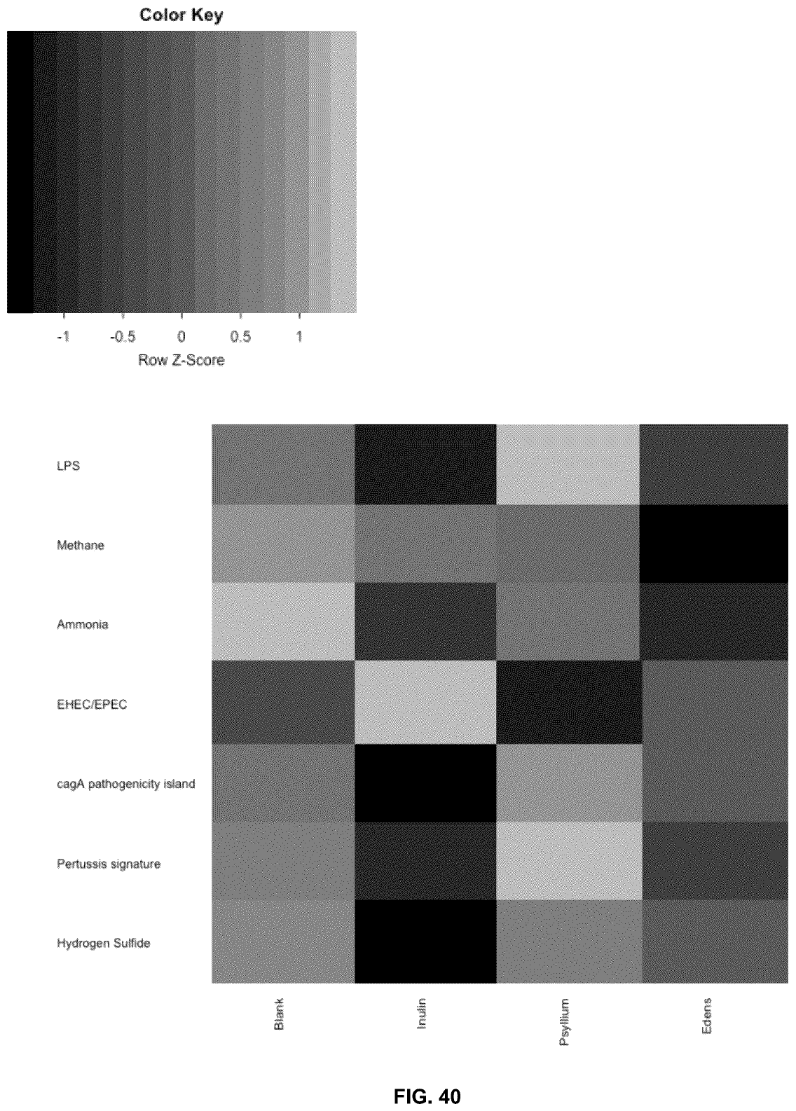

FIG. 40 illustrates JAN1000's effect on detrimental microbes. JAN1000 suppresses pathogenic and detrimental microbial pathways. The blank (i.e., stool samples that have not been treated with JAN1000) without a carbon source allows for opportunistic pathogenicity with lack of dietary fiber to protect the gut. Psyllium, as a mixed carbohydrate source, exacerbates a number of pathogenic pathways, by serving as a rich carbon source. Inulin, a simple carbohydrate source, generally drives the populations away from pathogenic phenotypes since it targets specific acetate and lactate producing bacteria. JAN1000, despite being a mixed dietary fiber source, still steers the microbiome in a positive direction by decreasing the expression of pathogenic signatures. JAN1000's unique mix of carbohydrates selectively targets the upregulation of beneficial bacteria while suppressing those that are detrimental to health.

In summary, JAN1000 serves as a rich source of diverse and unique dietary carbohydrates, primarily enriched in β-galactomannan, β-glucan, and resistant starch. This combination of carbohydrates yields enhanced benefit by augmenting the production of SCFA, MCFA and indoles. By feeding a specific niche of β-mannose-degrading, butyrogenic keystone bacteria, JAN1000 drives a healthy gut ecosystem. Additionally, JAN1000 promotes growth of a specific bacterium that elongates SCFA into MCFA, thus serving as a mechanistic bridge for enhanced MCFA production. JAN1000 upregulates beneficial microbial metabolic pathways by feeding keystone species and depleting opportunities for pathogenic bacteria. JAN1000 is effective when compared to inulin and Psyllium husk supplements, in improving metabolic, immune, and gut health at multiple levels of the microbiome-host interface.

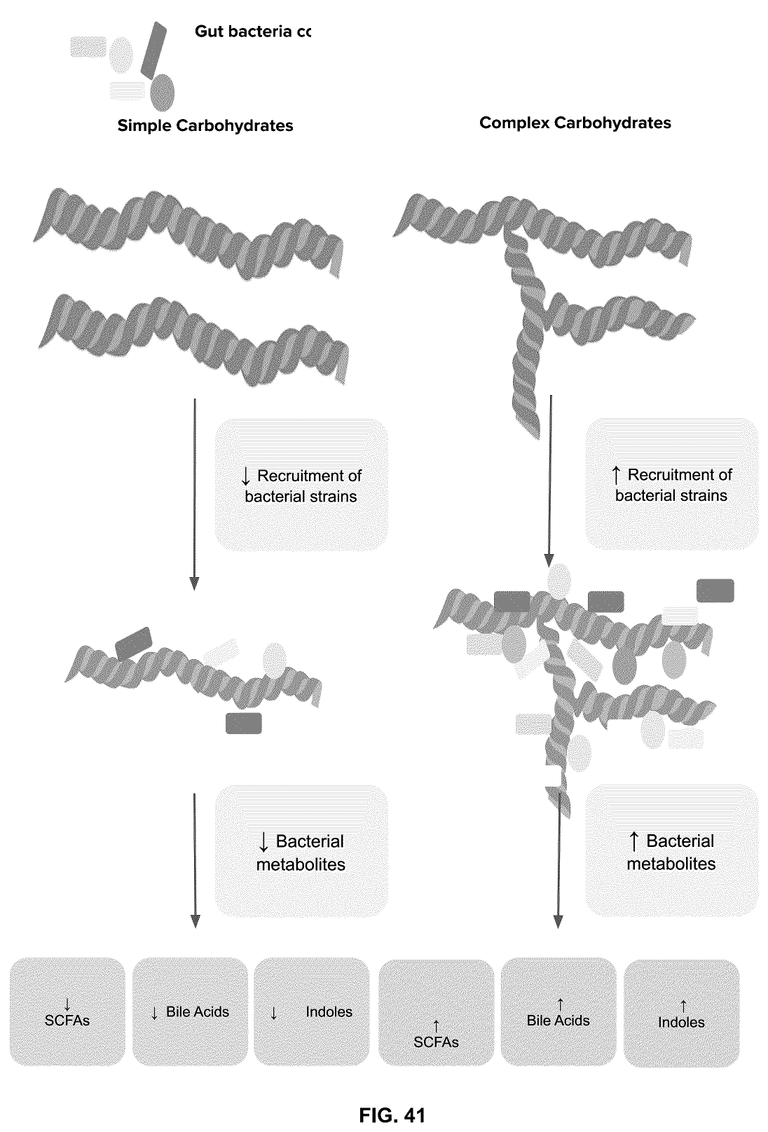

FIG. 41 illustrates how diverse monosaccharides that form complex polysaccharides have great potential to recruit fermentative bacterial consortiums. The recruitment of gut bacterial consortiums by dietary fiber is a function of neutral monosaccharide composition and linkages of these neutral sugars. Complexity of the prebiotic carbohydrate may have a critical impact on the abundance and variety of bacteria recruited to the colonic site of dietary fiber fermentation. JAN1000's branching complexity is predicted to be the driving cause of high fermentative potential and downstream bioactives.

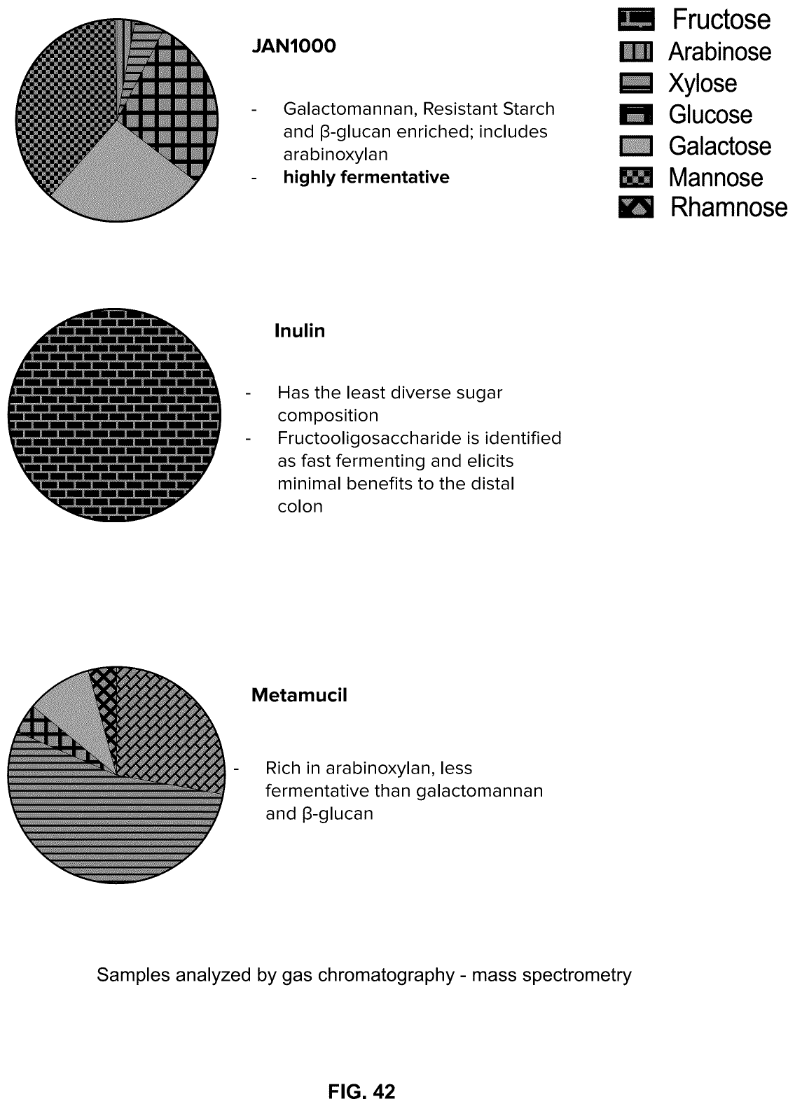

FIG. 42 illustrates comparisons of composition of JAN1000 to those of supplements Inulin and Metamucil®. JAN1000 is composed of diverse and highly fermentative neutral monosaccharides. By comparison, inulin has a less diverse sugar composition.

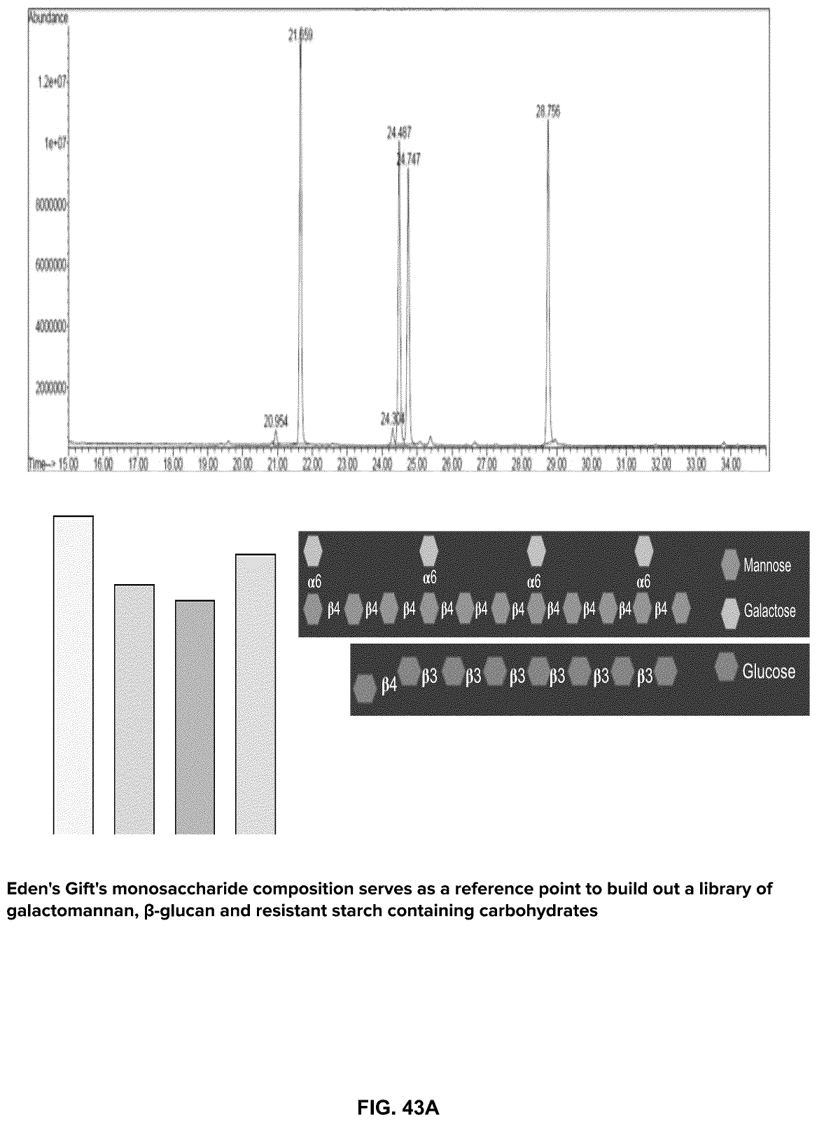

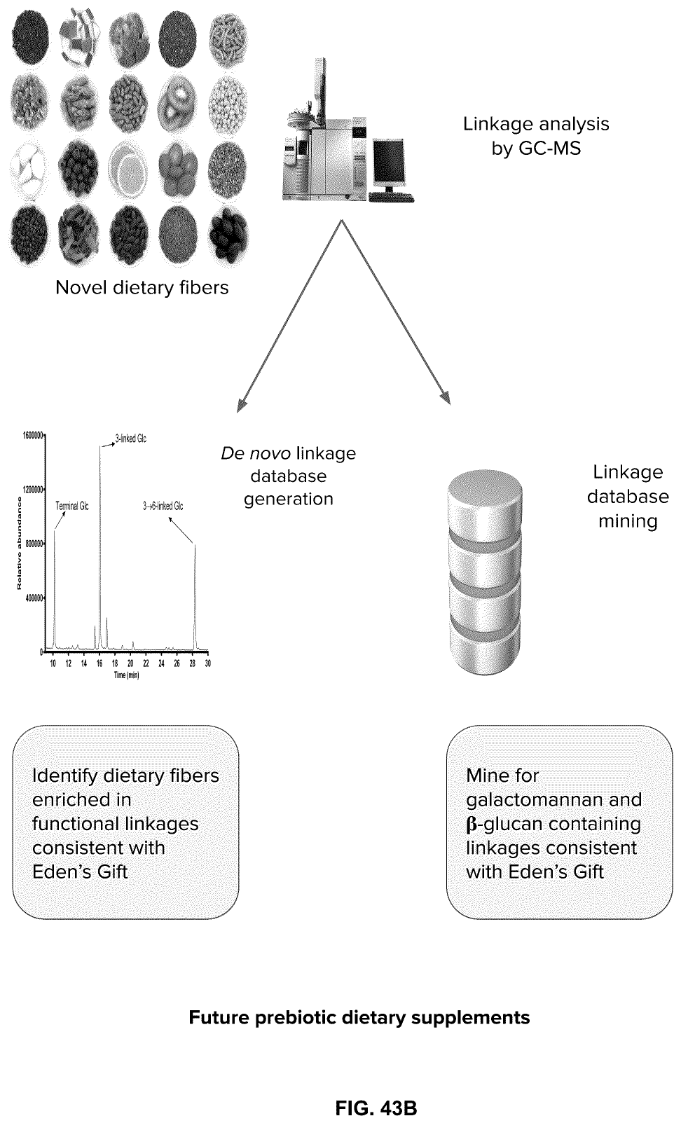

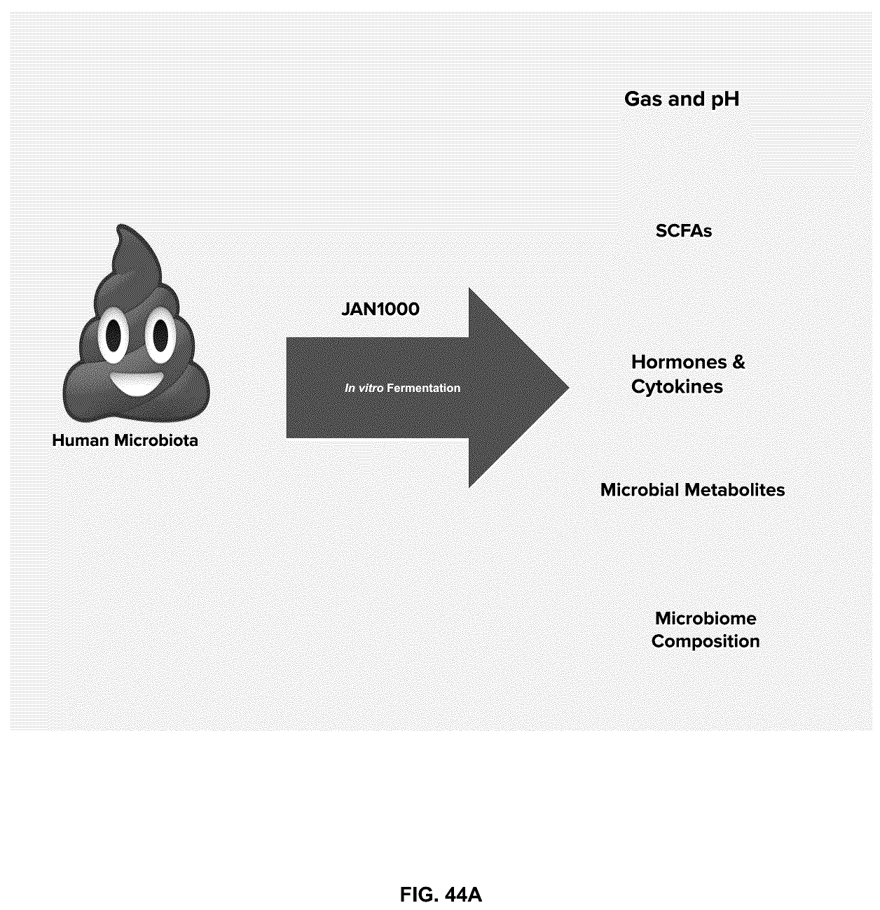

FIGS. 43A-43B illustrate that JAN1000 is rich in fermentative polysaccharides like galactomannan, β-glucan and resistant starch type 2. JAN1000 monosaccharide composition serves as a reference point to build out a library of galactomannan, β-glucan and resistant starch containing carbohydrates.

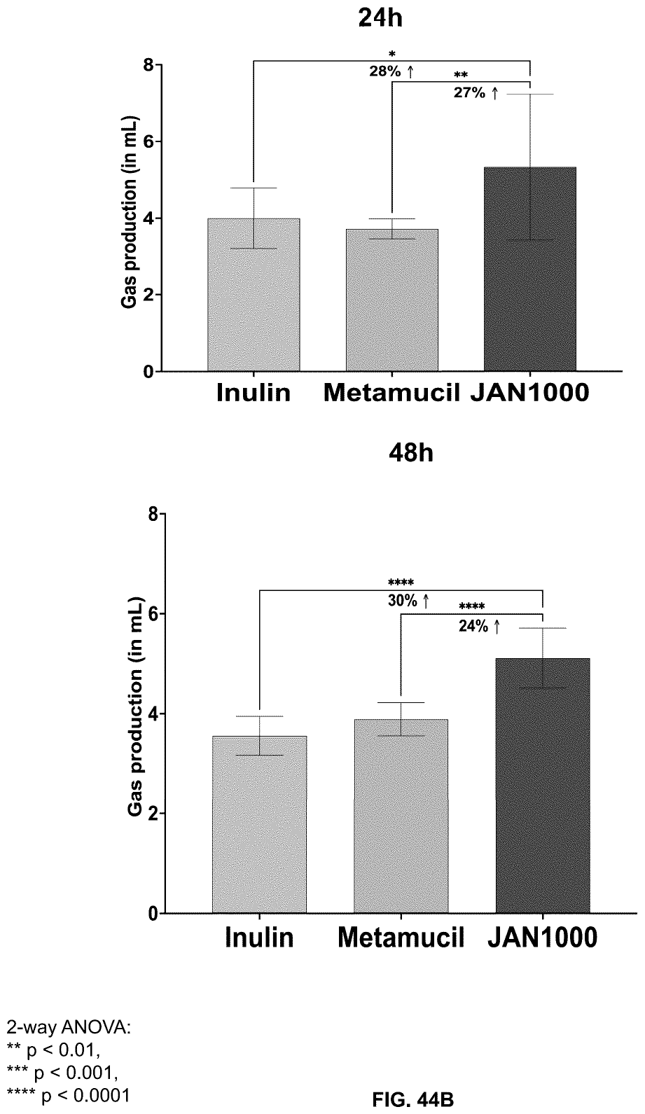

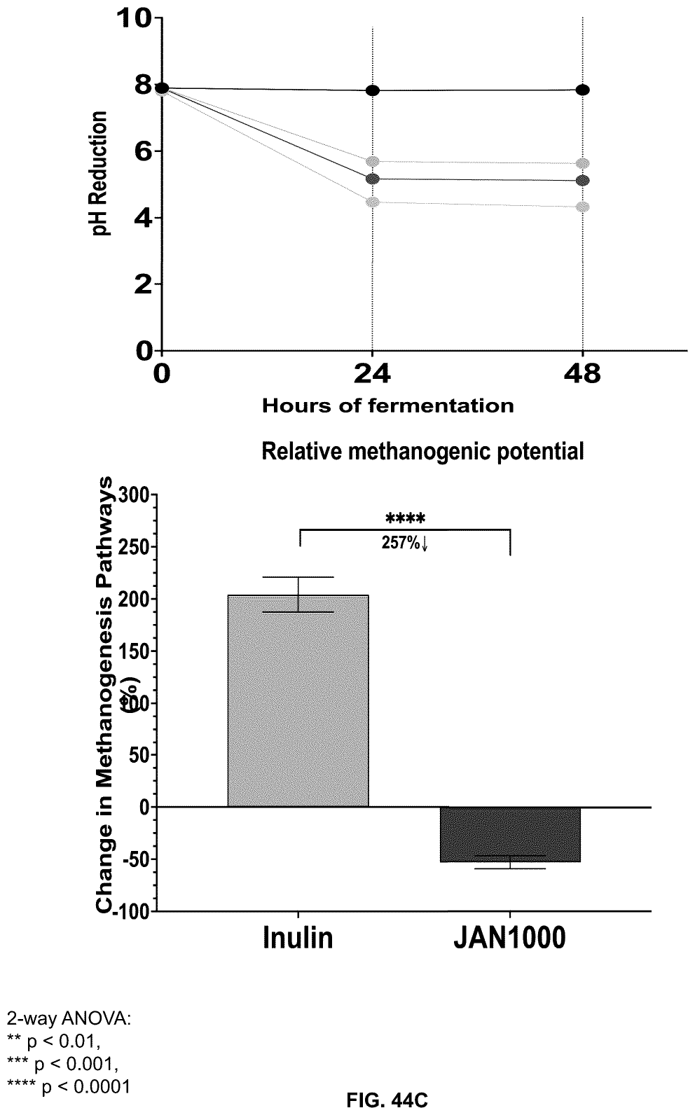

FIGS. 44A-44C illustrate that JAN1000 elicits a stronger fermentation profile than inulin and Metamucil®. Gas production is a surrogate marker of fermentation. On average, JAN1000 produces 29% more gas when compared to inulin and 25.5% more gas when compared to Psyllium husk. pH reduction reflects an amount of acid produced during fermentation. Inulin produces the greatest reduction in pH, Additionally, JAN1000 produces the largest amount of short-chain fatty acids. These increases in production are in effect after 24 and 48 hours of fermentation.

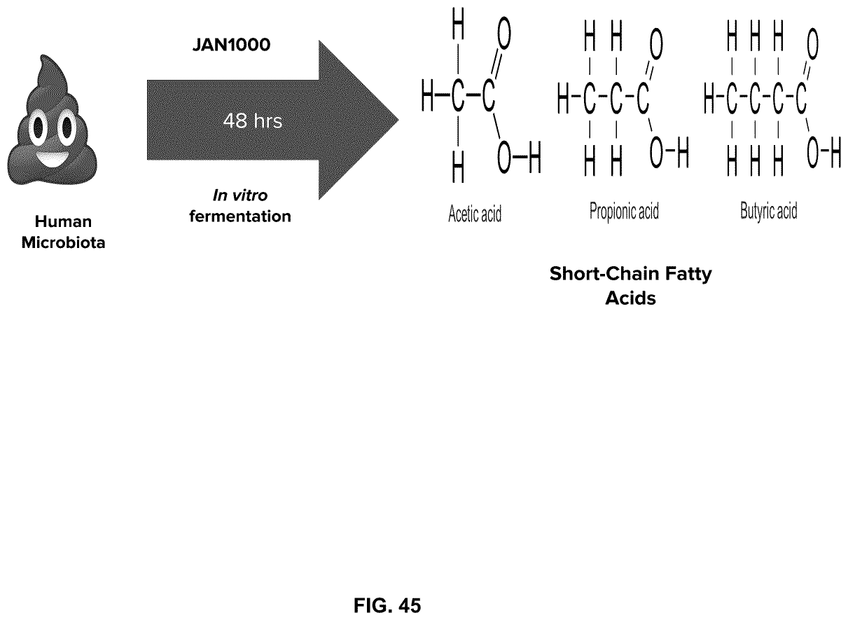

FIG. 45 illustrates that a disclosed experiment compares the propensity for a donor's microbiota to produce short-chain fatty acids (SCFAs) from JAN1000, inulin, and Metamucil®. Fermentation of JAN1000 yields greater SCFA than inulin and Metamucil®. In this experiment, three donor stool samples were placed into in vitro fermentation with JAN1000, inulin, and Metamucil. Post fermentation, samples were analyzed by a semi-quantitative thin layer chromatography (TLC) method, measured in triplicate. Then, semi-quantitation was performed. This experiment tested a fermentation environment rather than using fecal samples.

SCFAs serve as bioactives in the gut that are used by enterocytes as energy sources and by enteroendocrine cells to elicit hormonal responses. A circulating concentration of SCFAs that make it into the blood stream would likely be underestimated due to most of the SCFAs being metabolized or cleared by the gut cells. Similarly, fecal SCFA may not accurately describe the true effect of local SCFA production in the colon. SCFA are surrogate biomarkers of fermentation and may correlate strongly with host biomarkers such as GLP-1.

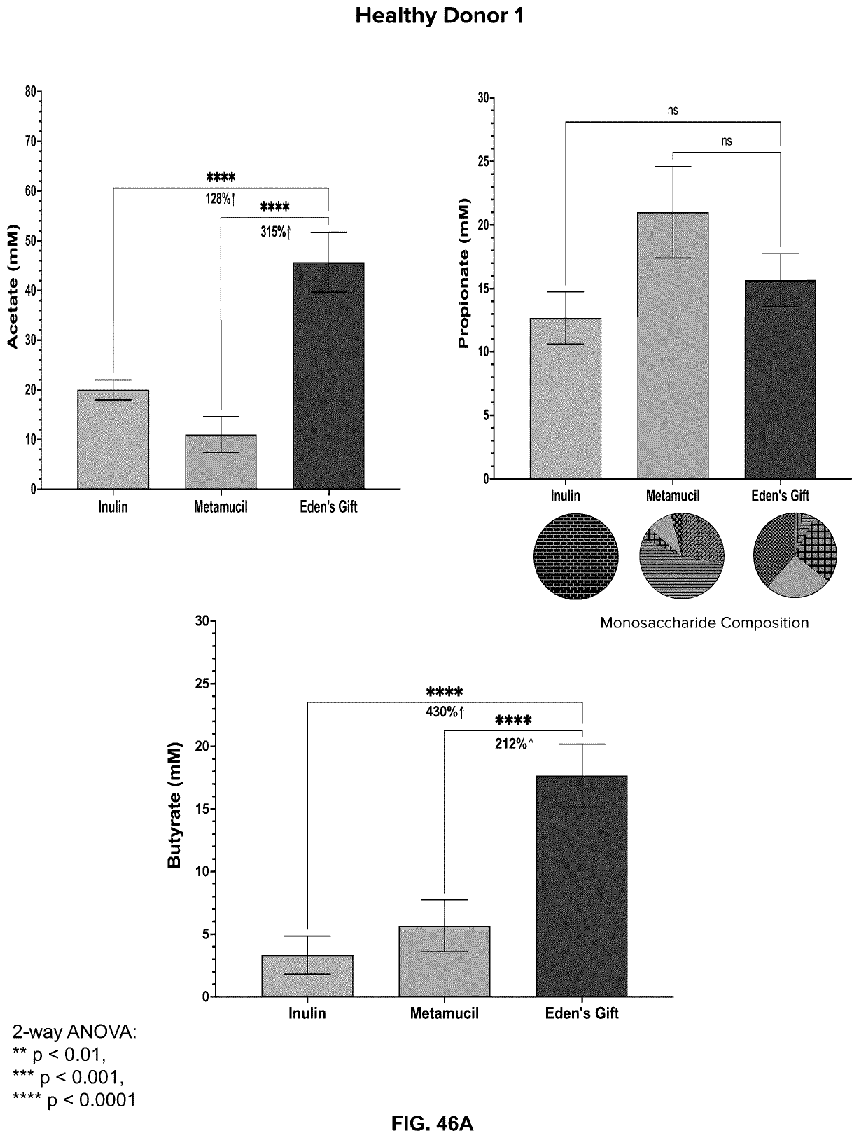

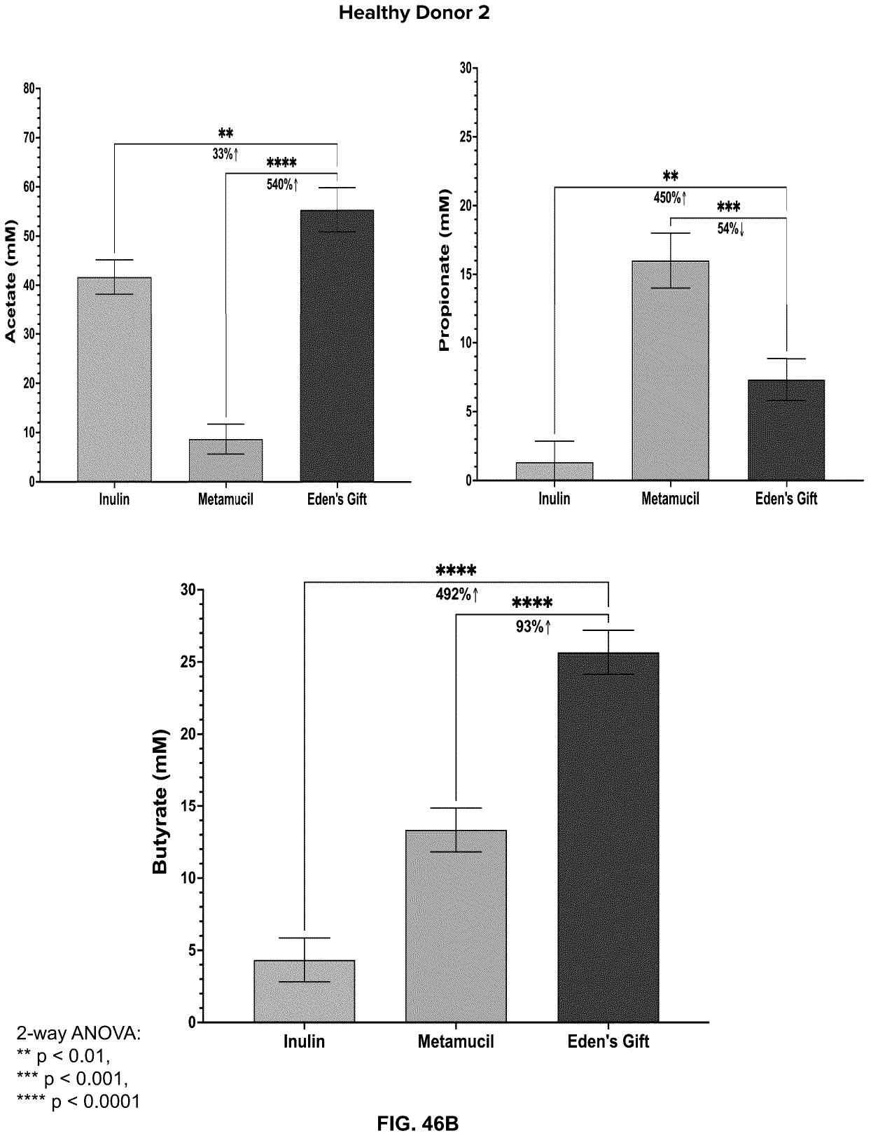

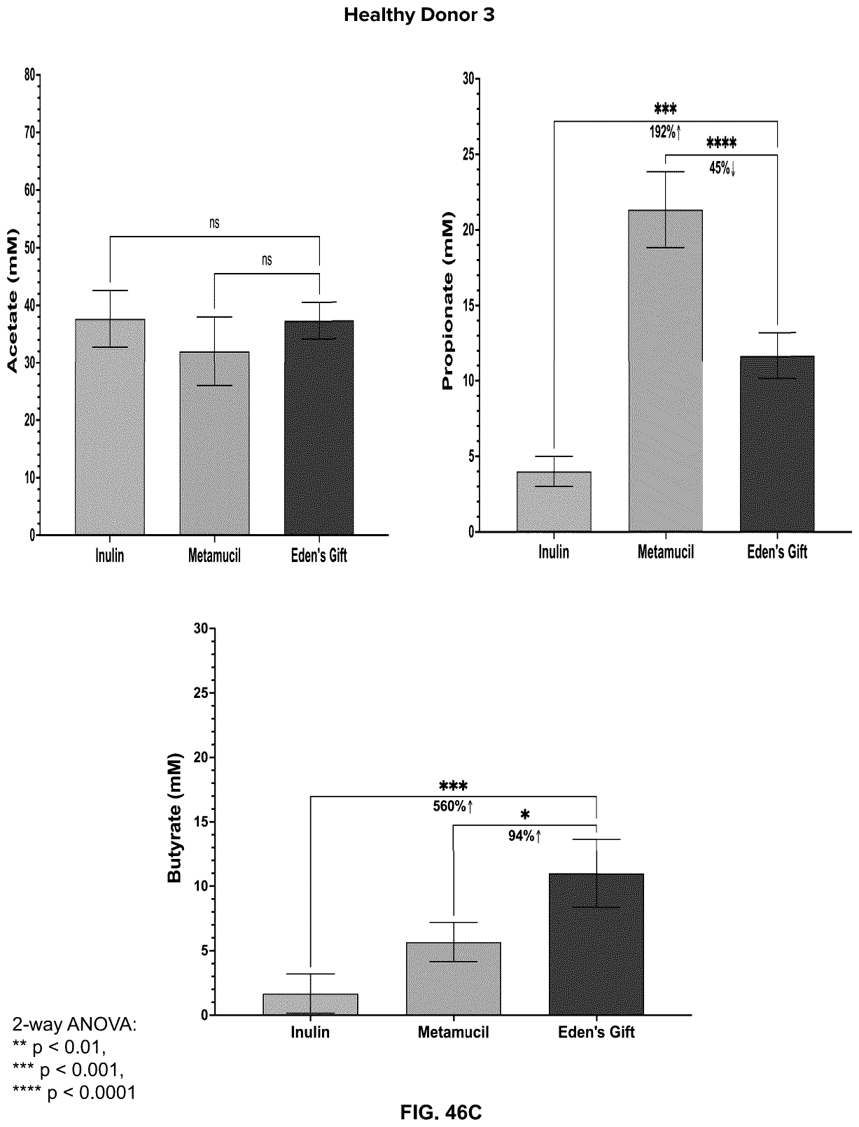

FIGS. 46A-46C illustrate an experiment illustrating effects of JAN1000, inulin, and Metamucil® on production of butyrate and acetate in healthy patients. FIGS. 46A-46C illustrate that JAN1000 produces more butyrate and acetate than inulin and METAMUCIL®. In the disclosed experiment, JAN1000 produces more acetate than inulin and Metamucil® in Donors 1 & 2 but not in Donor 3. Metamucil® produces more propionate but less acetate than inulin and JAN1000. JAN1000 is superior in producing butyrate than inulin and Metamucil®. Butyrate production is a function of carbohydrate complexity (monosaccharide composition & polysaccharide branching). JAN1000 showed more fiber diversity than inulin or Metamucil®.

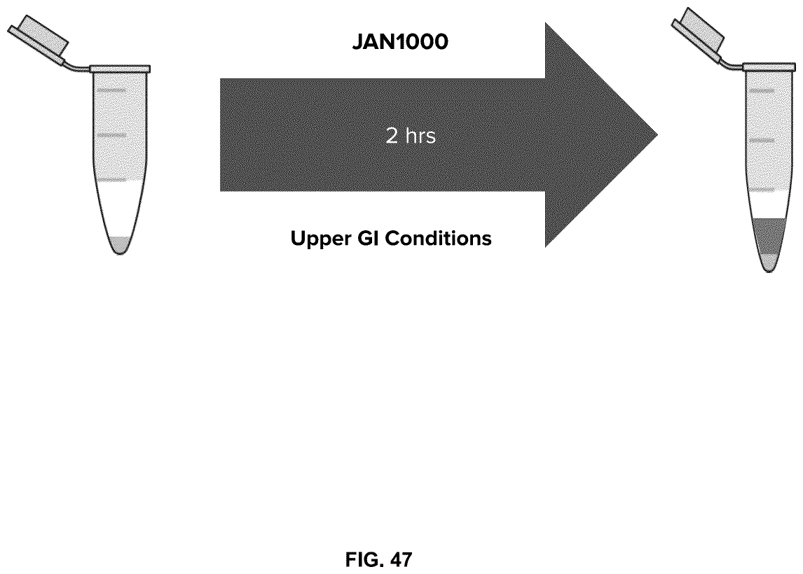

FIG. 47 illustrates an experiment in which anti-cholesterol potential of JAN1000 is compared to that of inulin and Metamucil® in an in vitro model. JAN1000 exhibited a greater potential to reduce cholesterol in the upper GI than inulin and Metamucil®. A meal mixture (cholesterol, cholesteryl ester, protein, fats, and carbohydrates) was suspended in upper gastrointestinal conditions. Fibers (JAN1000, inulin, and Metamucil®) were added to the mixture and allowed to incubate for two hours at small intestinal conditions. The final concentration of cholesterol was measured via colorimetric enzyme-linked immunoassay (ELISA) against a blank sample containing only the meal mixture.

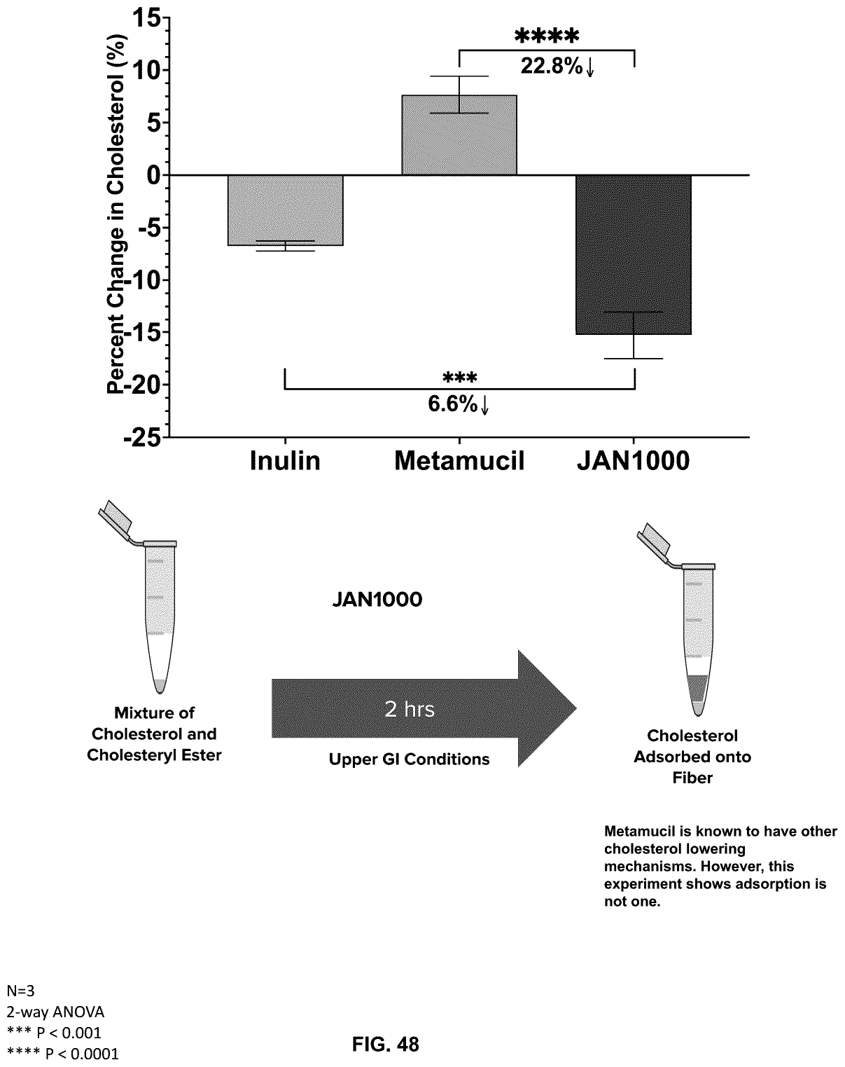

FIG. 48 illustrates an experiment comparing the reduction of cholesterol by JAN1000 to that of inulin and Metamucil®. The experiment showed that JAN1000 reduces cholesterol more effectively than inulin and Metamucil. In particular, JAN1000 reduces by 6.6% more cholesterol than inulin and by 22.8% more than Metamucil via adsorption. Unlike inulin and JAN1000, Metamucil increases the cholesterol concentration by 7.7% in this assay. This is likely attributed to Metamucil's propensity to absorb water while excluding lipid adsorption. Unlike soluble inulin and insoluble Metamucil, JAN1000 is comprised of a combination of soluble and insoluble fibers that serve multiple mechanisms for cholesterol reduction.

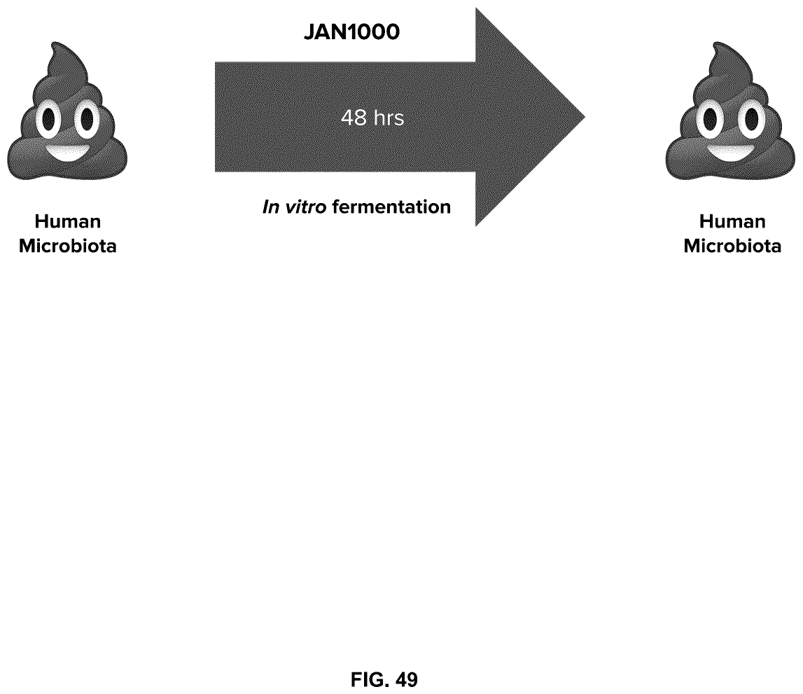

FIG. 49 illustrates an experiment to evaluate the modulatory effect of JAN1000 on intestinal microbiome. JAN1000 promotes protective bacterial genera & decreases pathogens. The experiment uses one healthy donor stool sample. In further experiments, samples from additional donors treated with JAN1000, insulin and Metamucil® may be used. Samples are exposed to JAN1000 for 48 hours via in vitro fermentation. Samples were analyzed pre and post JAN1000 fermentation.

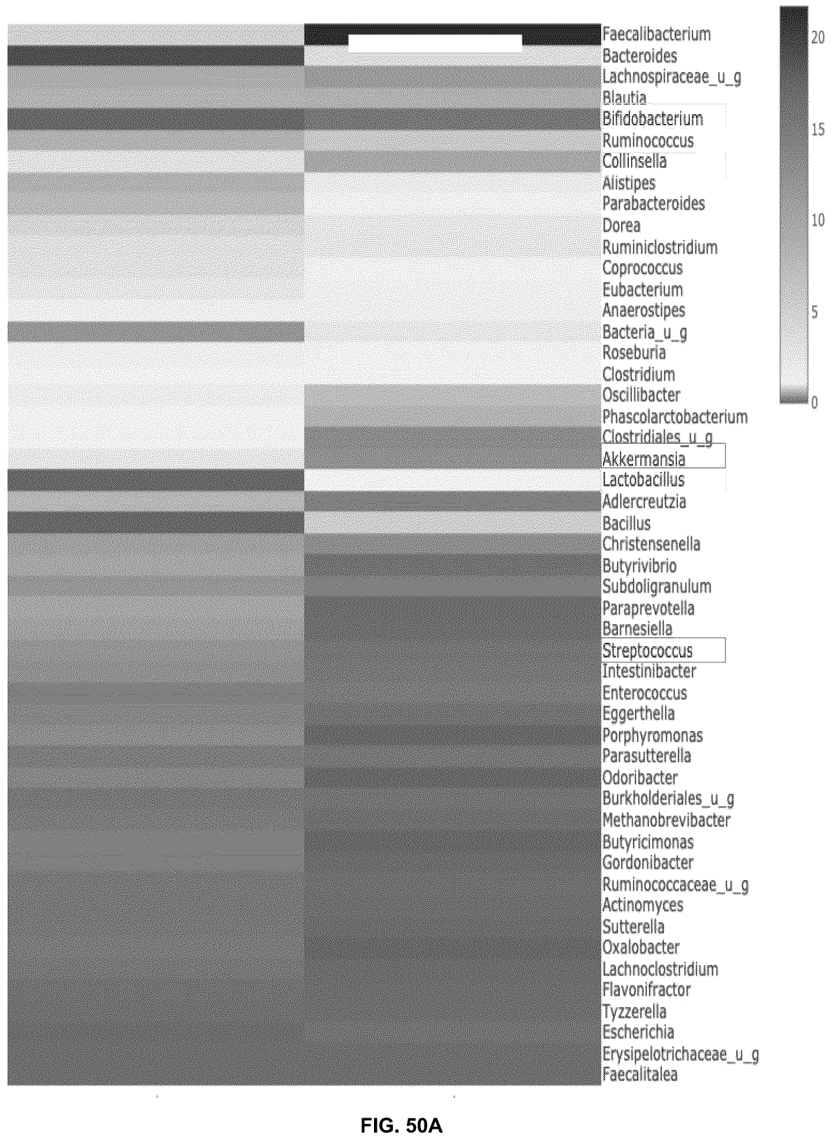

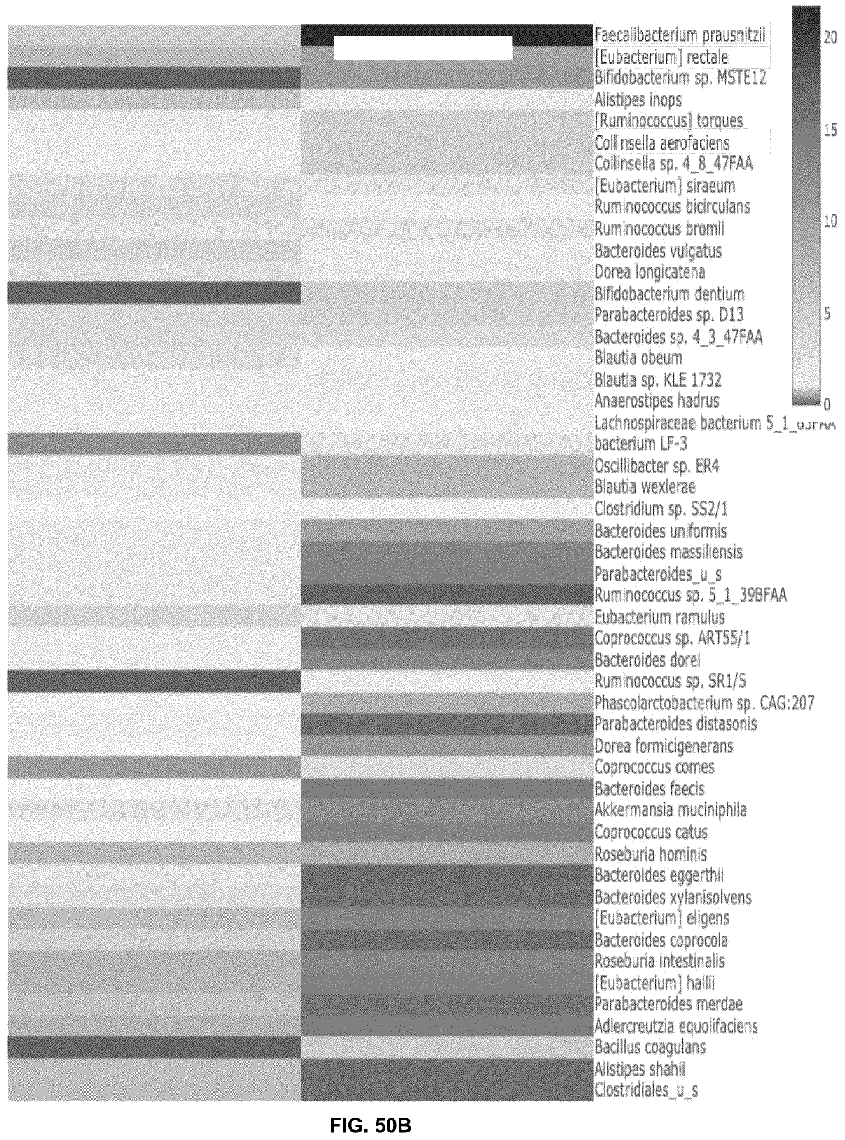

FIGS. 50A-50B illustrate experimental results showing JAN1000's effects on populations of various microorganisms. The results show that JAN1000 promotes the growth of beneficial keystone bacteria while reducing potentially pathogenic populations.

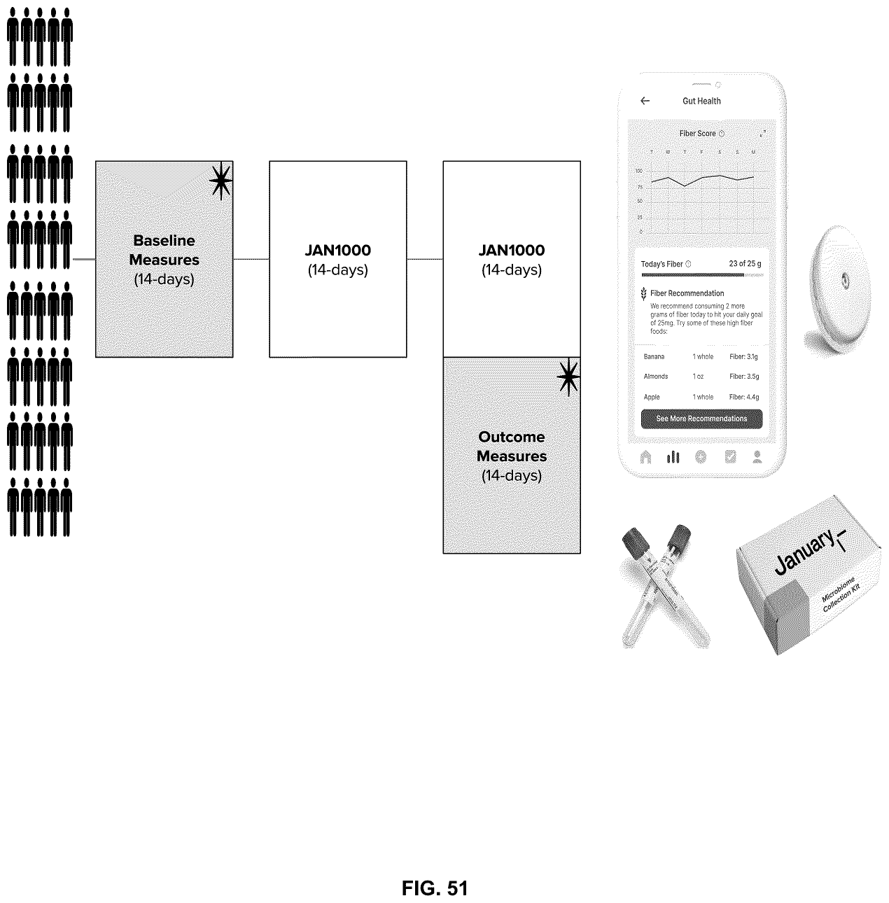

FIG. 51 illustrates an experiment which is a proposed human clinical study designed to test product quality and satisfaction, as well as to get an early read on satiety, blood glucose, and improvements to gut and metabolic health. The experiment tested healthy participants and participants with prediabetes and type 2 diabetes. Participants are encouraged to experiment with standardized and favorite foods with the addition of the supplement JAN1000. Participants all eat one standard, controlled meal to measure postprandial glucose recovery. Participants are encouraged to participate in sample collection. In the experiment, there may be 40-50 participants. The experiment may be a pre/post study, so outcomes will be baselined to a period of 14 days before intervention with JAN1000 between and within each participants and groups of participants. The experiment may categorize subjects as healthy, prediabetes, or type 2 diabetes.

Each serving of JAN1000 has 15 g of dietary fiber, 25 billion CFU of probiotics, and 1.5 g of concentrated polyphenols. Participant data will be captured using a mobile application coupled to CGM. Blood and stool samples will be collected for further analyses. Additionally, questionnaires will be sent out to collect responses around satiety, gut & metabolic health, and satisfaction of JAN1000.

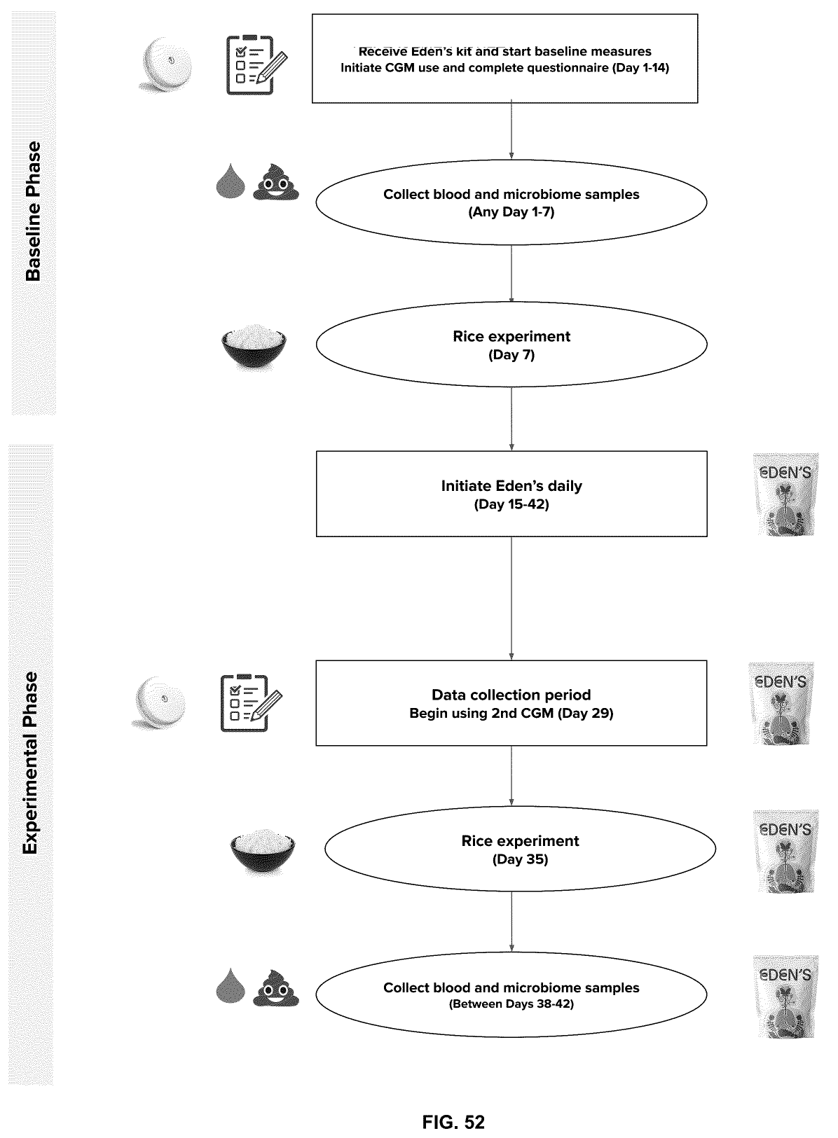

FIG. 52 illustrates a flow chart of the experiment. The experiment may be conducted over a period of 42 days. In a first two-week phase, participants collects physiological data relevant to anticipated intervention from JAN1000. The experiment may collect continuous glucose monitoring (CGM), blood, stool, and questionnaire data. In an experimental phase, which may last four weeks, participants will start taking JAN1000 and logging when they take JAN1000. In a data collection phase, which may last two weeks, participants will again collect physiological data alongside supplementing with JAN1000. The physiological data may include CGM, blood, stool, and questionnaire data. The experiment may include a rice experiment. The rice experiment includes participants eating a controlled meal in a fasted state and then measuring their postprandial blood glucose curves. Will be used to compare glucose curve recovery with and without JAN1000.

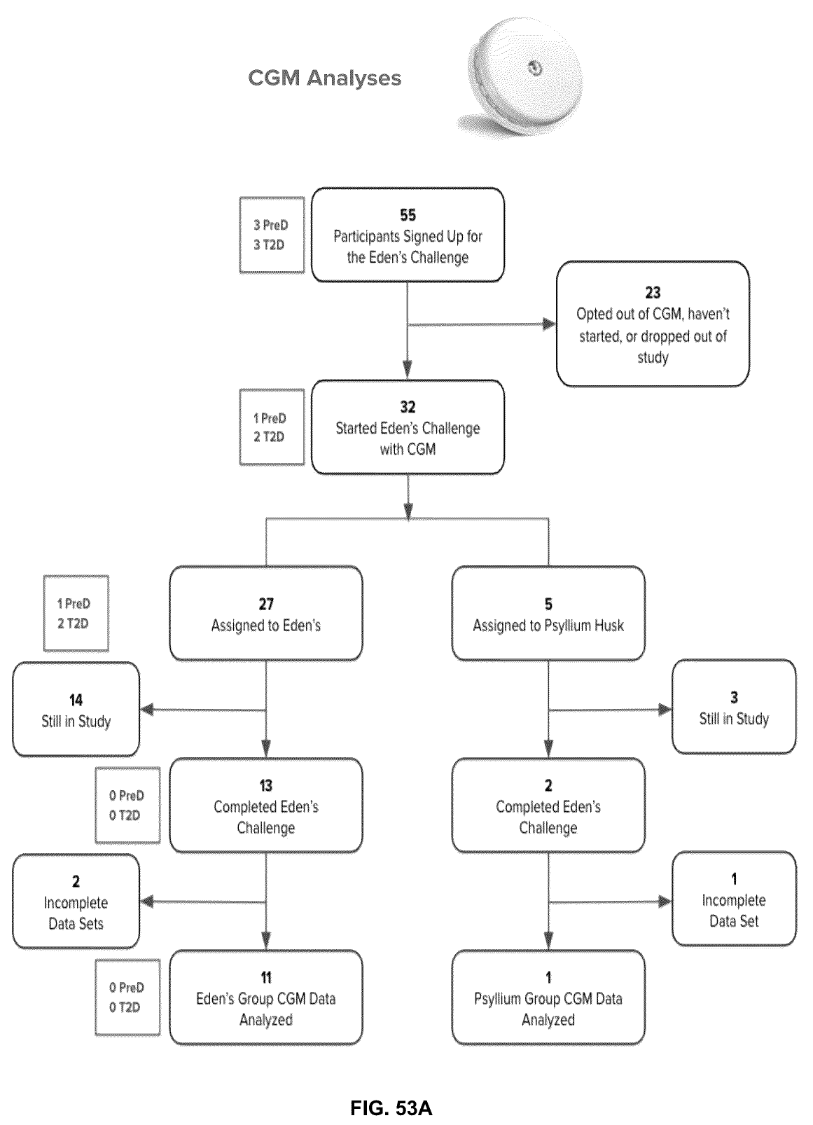

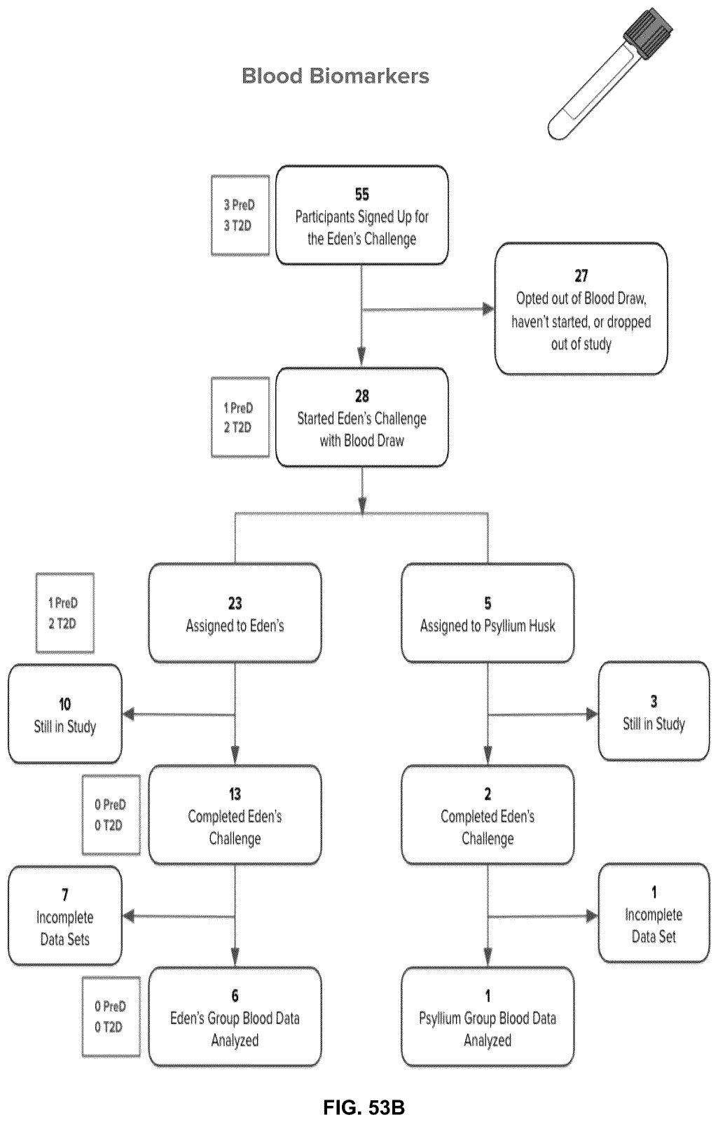

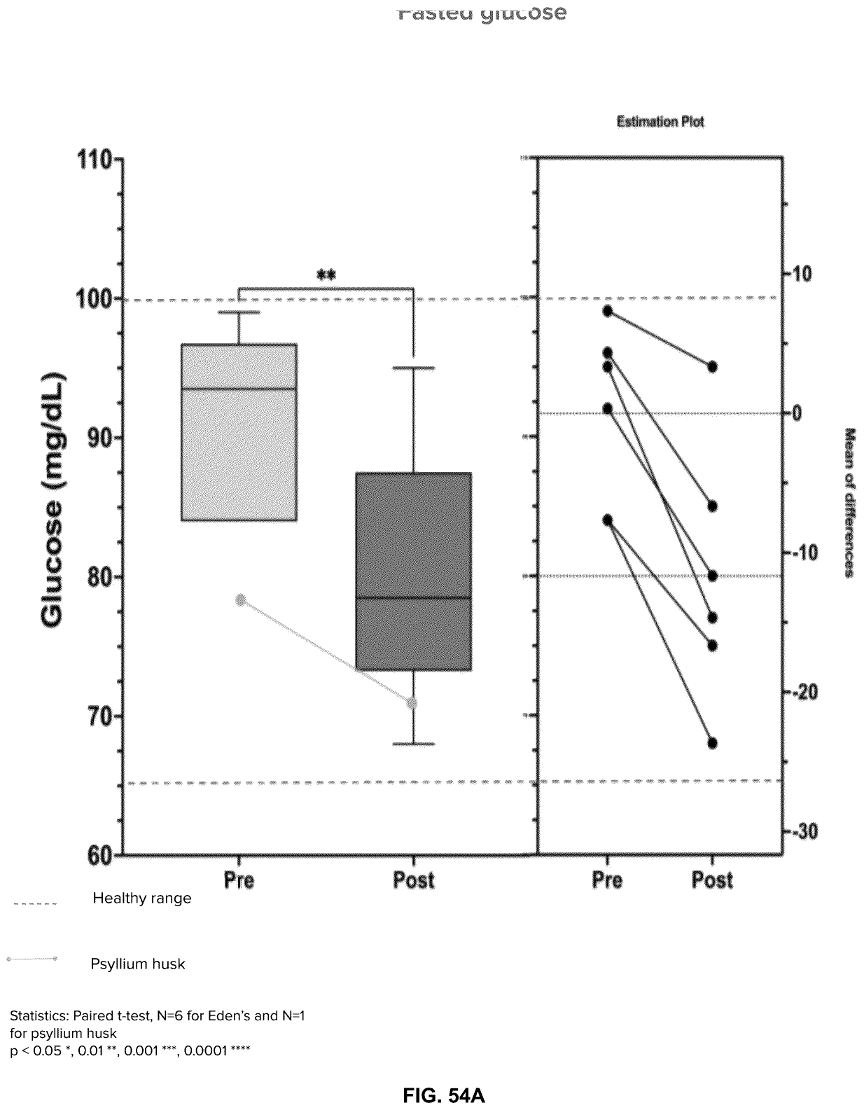

FIGS. 53A-53B illustrate an experimental design for collecting samples and testing them with the supplement JAN1000.

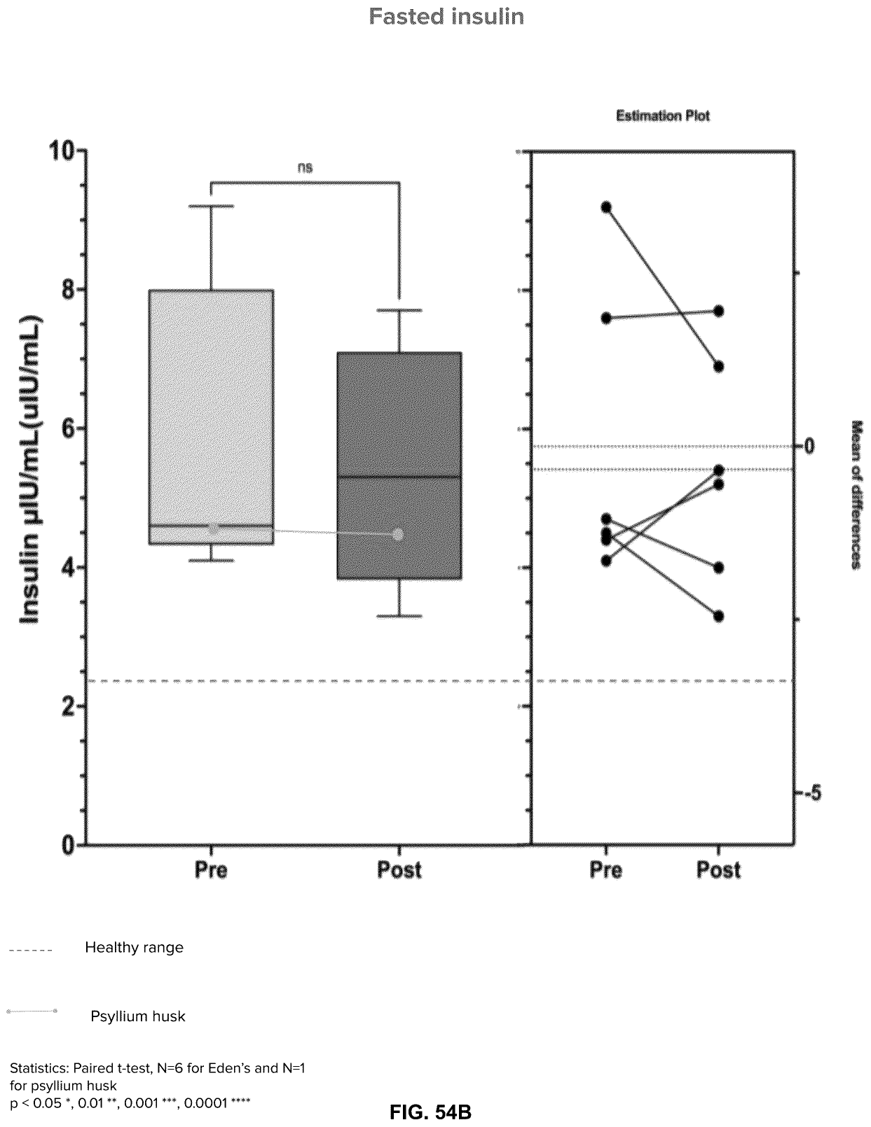

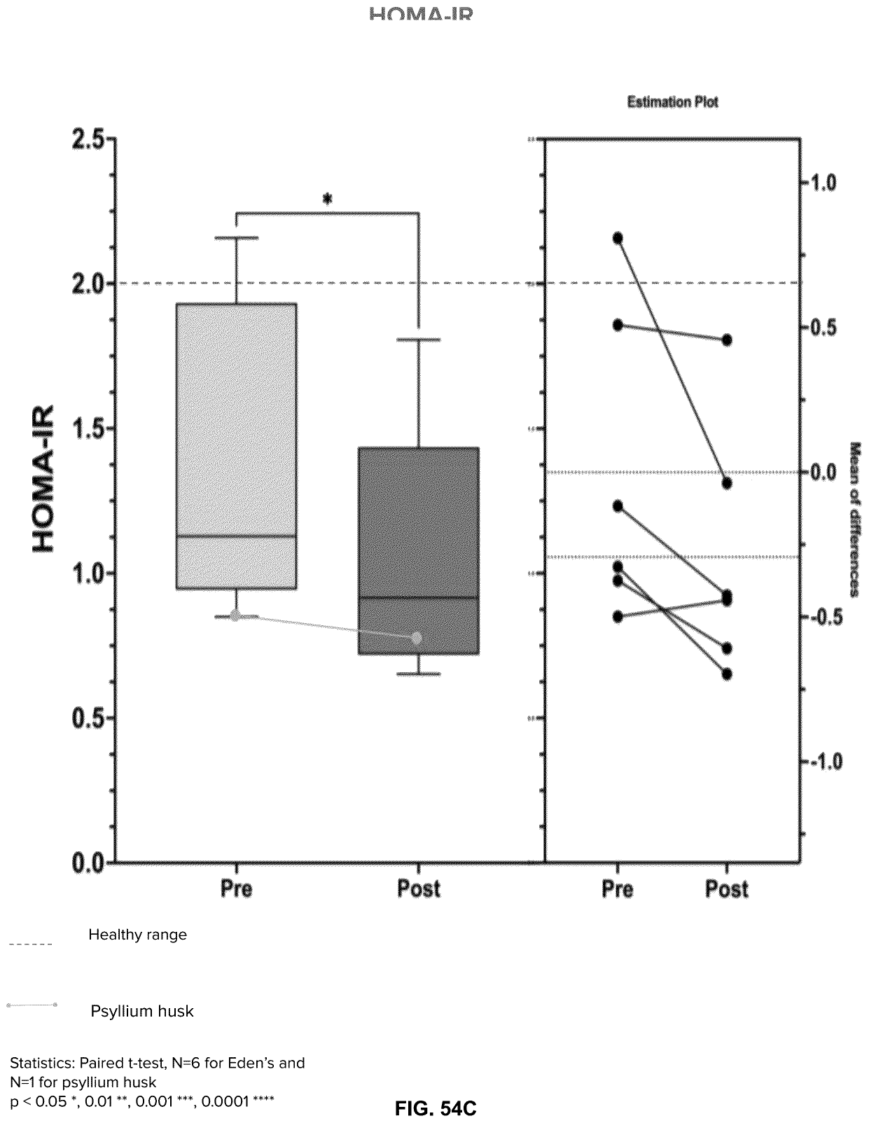

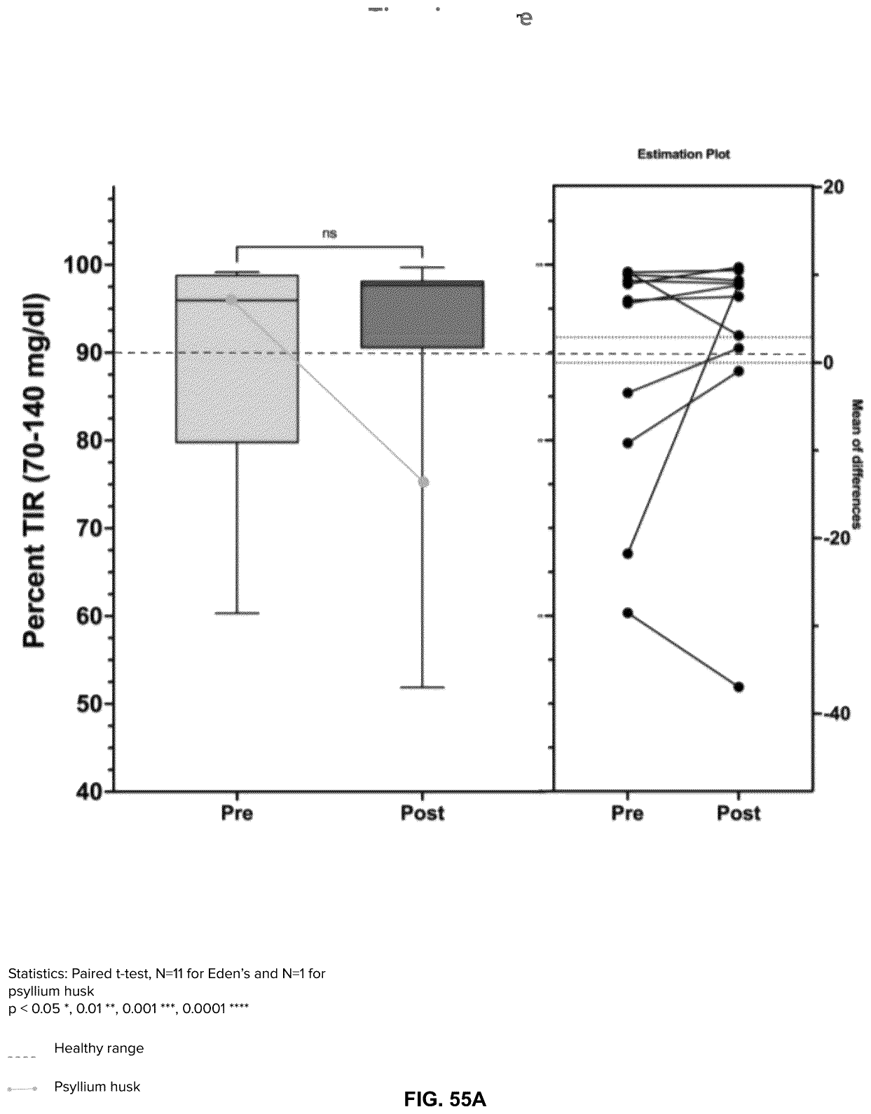

FIGS. 54A-54C illustrate results showing JAN1000 improves overall glucose homeostasis and insulin sensitivity. The left, middle, and right graphs show estimation plots respectively comparing changes in fasted glucose levels, fasted insulin levels, and HOMA-IR (a derived number from fasted glucose and insulin levels) pre- and post-treatment with Psyllium husk and JAN1000. The HOMA-IR number is clinically validated and represents insulin sensitivity. Compared to Psyllium husk, the left plot shows a more rapid decrease in glucose levels. The plots illustrate that the mean value post-treatment is lower than that pre-treatment, indicating improved homeostasis and insulin sensitivity.

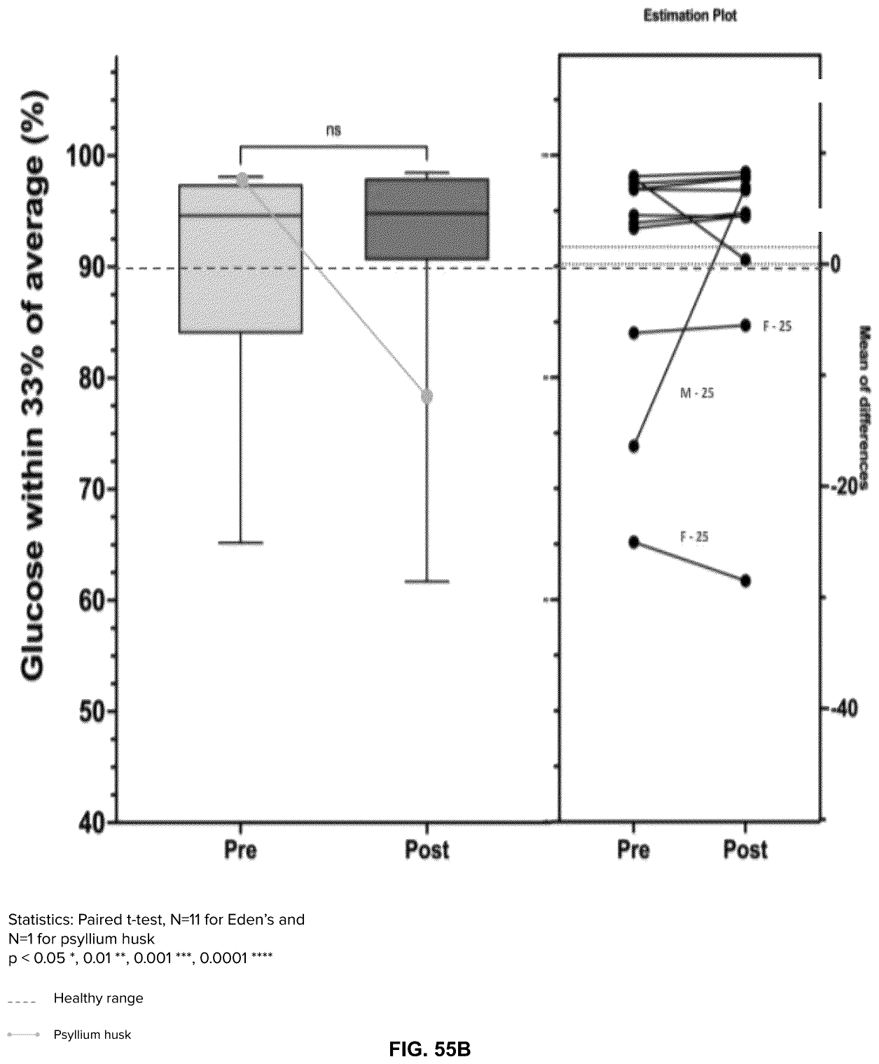

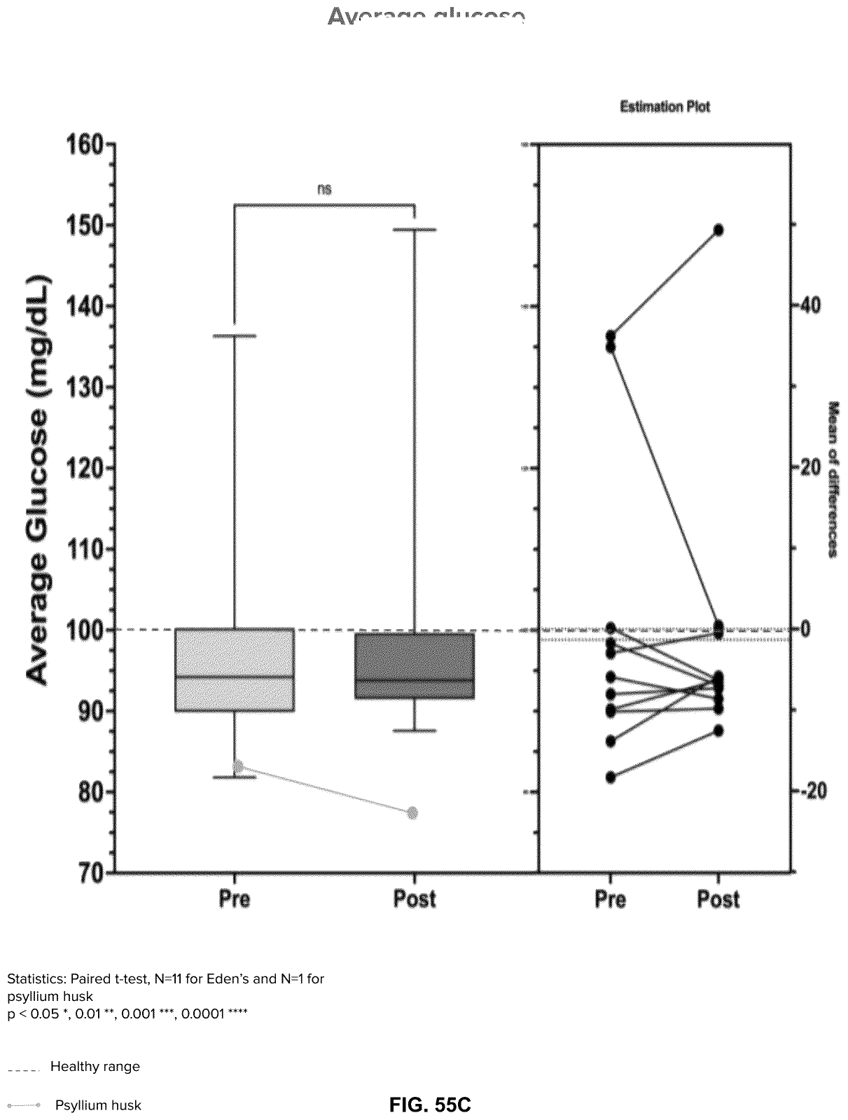

FIGS. 55A-55C illustrate a comparison of Psyllium husk with JAN1000 on time-in-range. Unlike Psyllium husk, JAN1000 improves time-in-range with less daily variability exhibited than Psyllium husk.

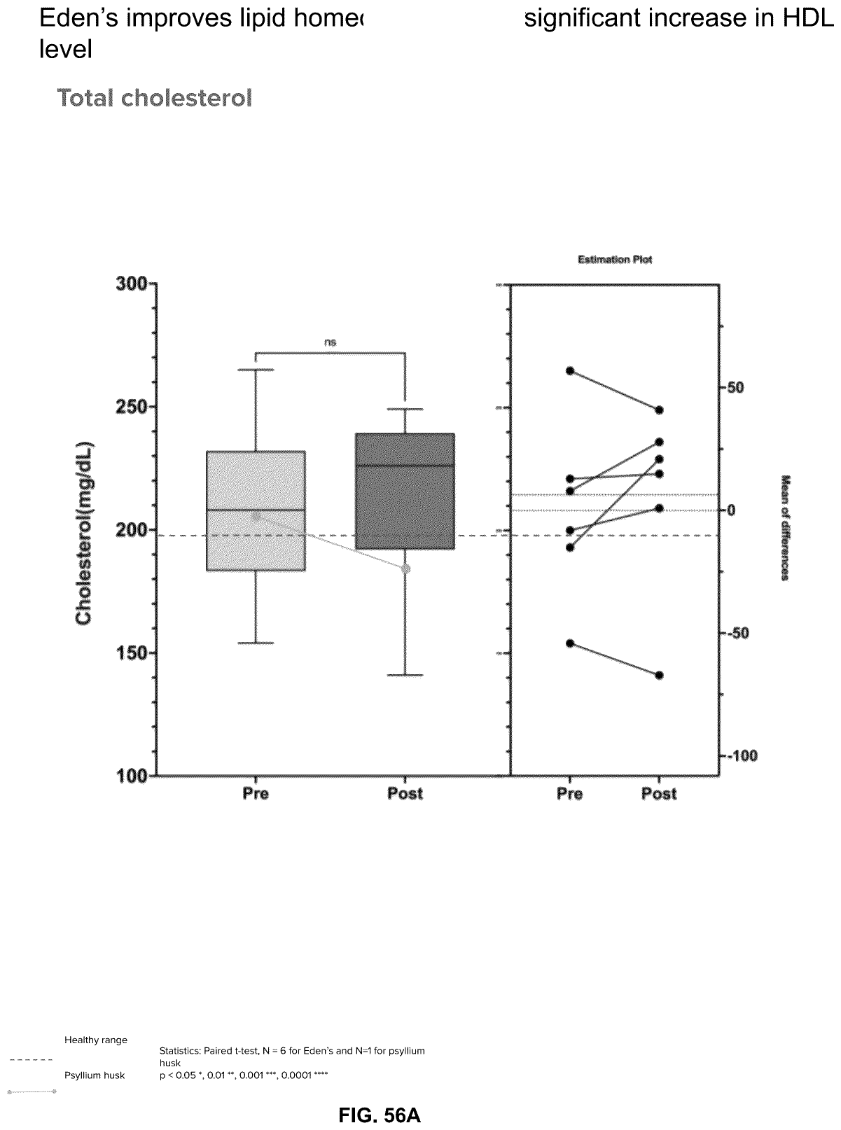

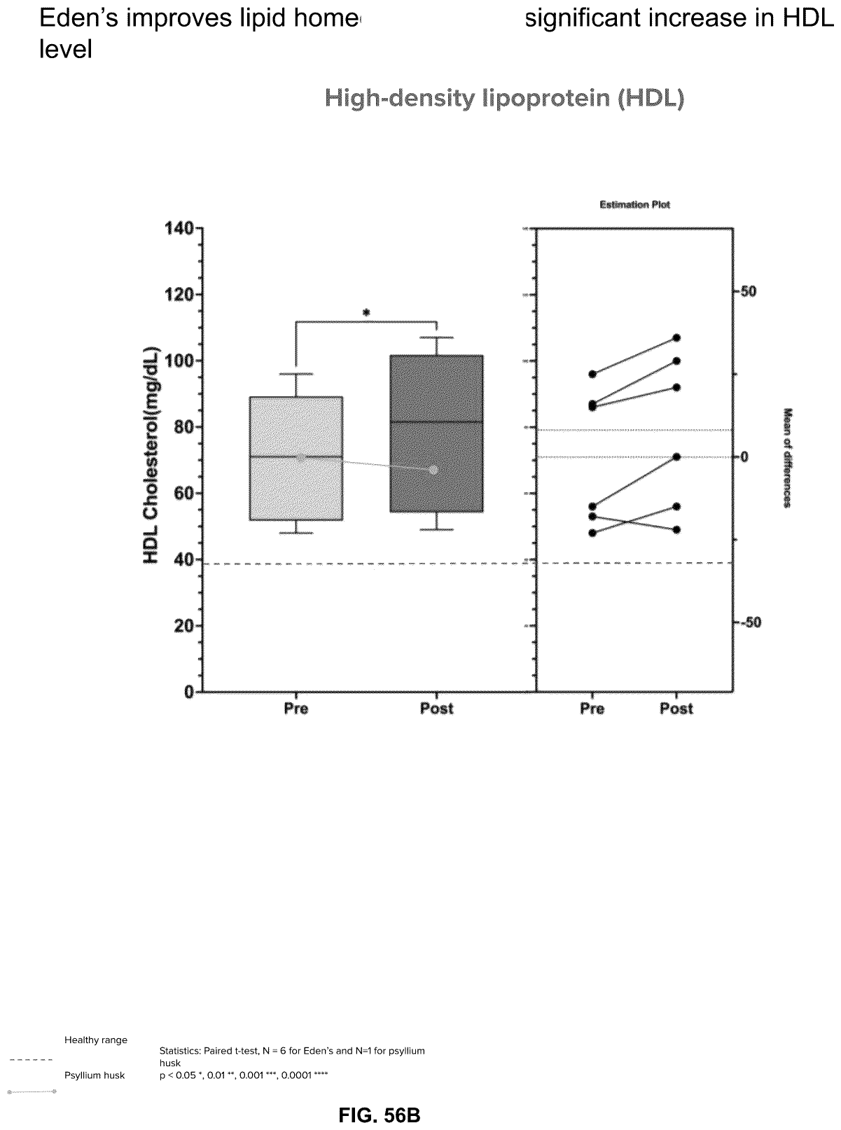

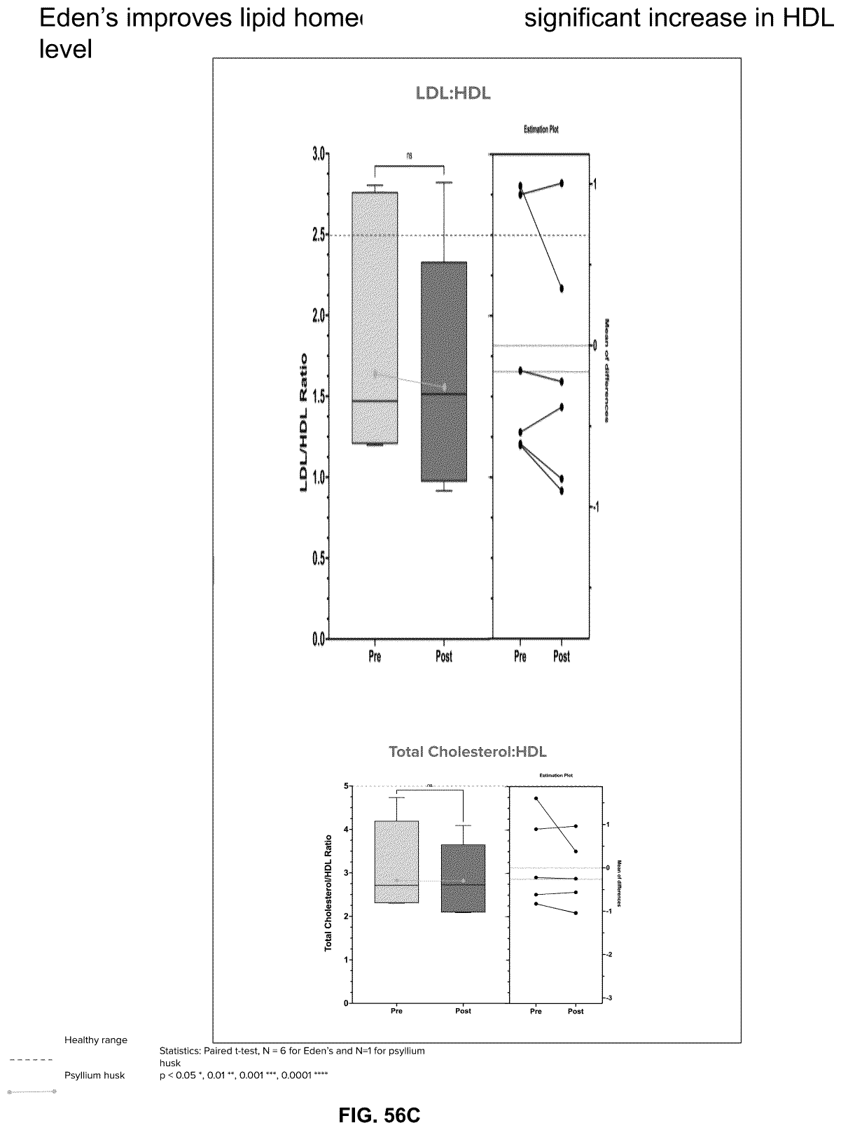

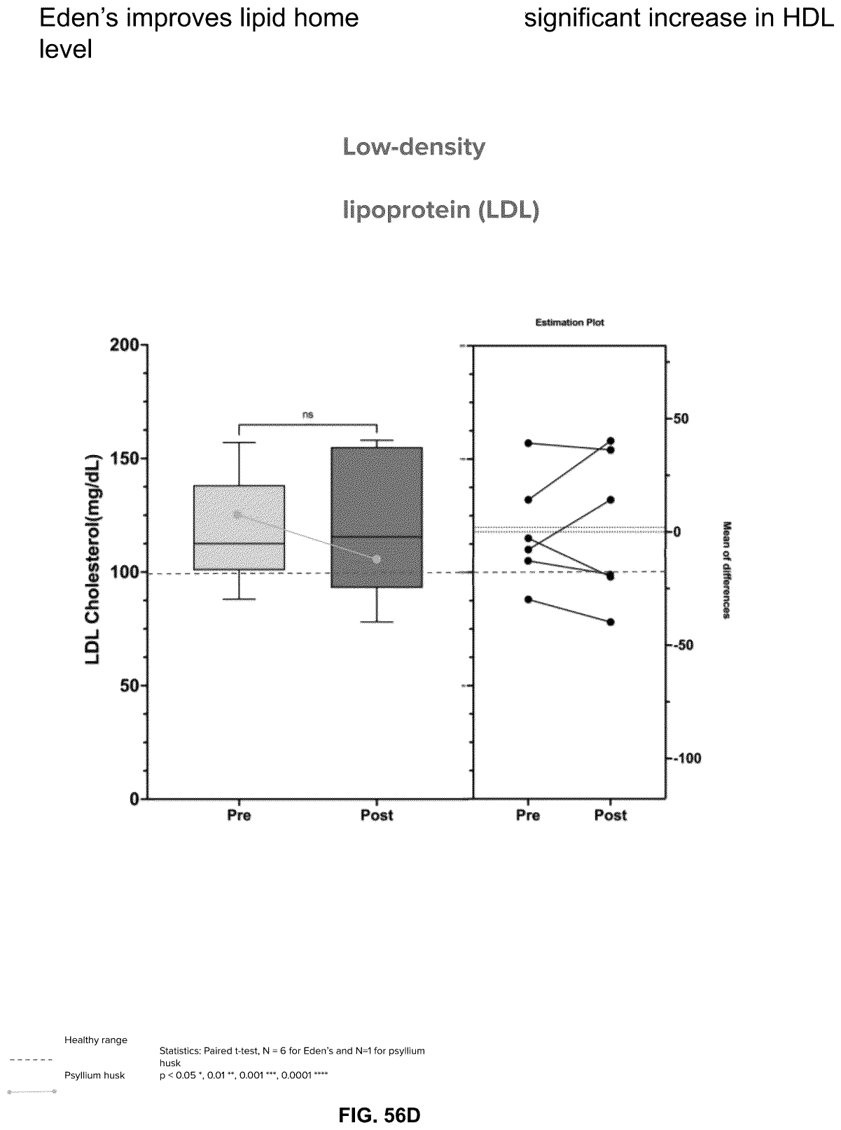

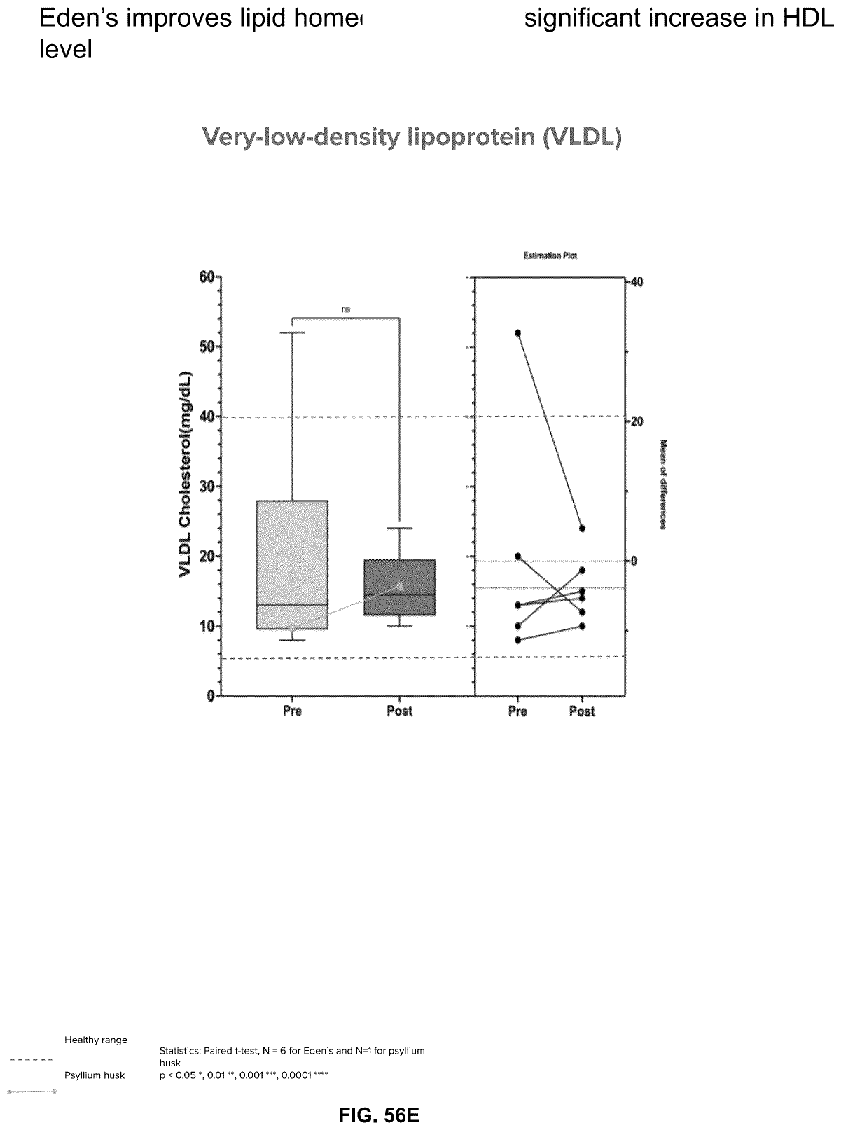

FIGS. 56A-56E illustrate comparative effects of JAN100 on lipid homeostasis. The plots show that JAN1000 improves lipid homeostasis with a significant increase in HDL level, when compared to Psyllium husk.

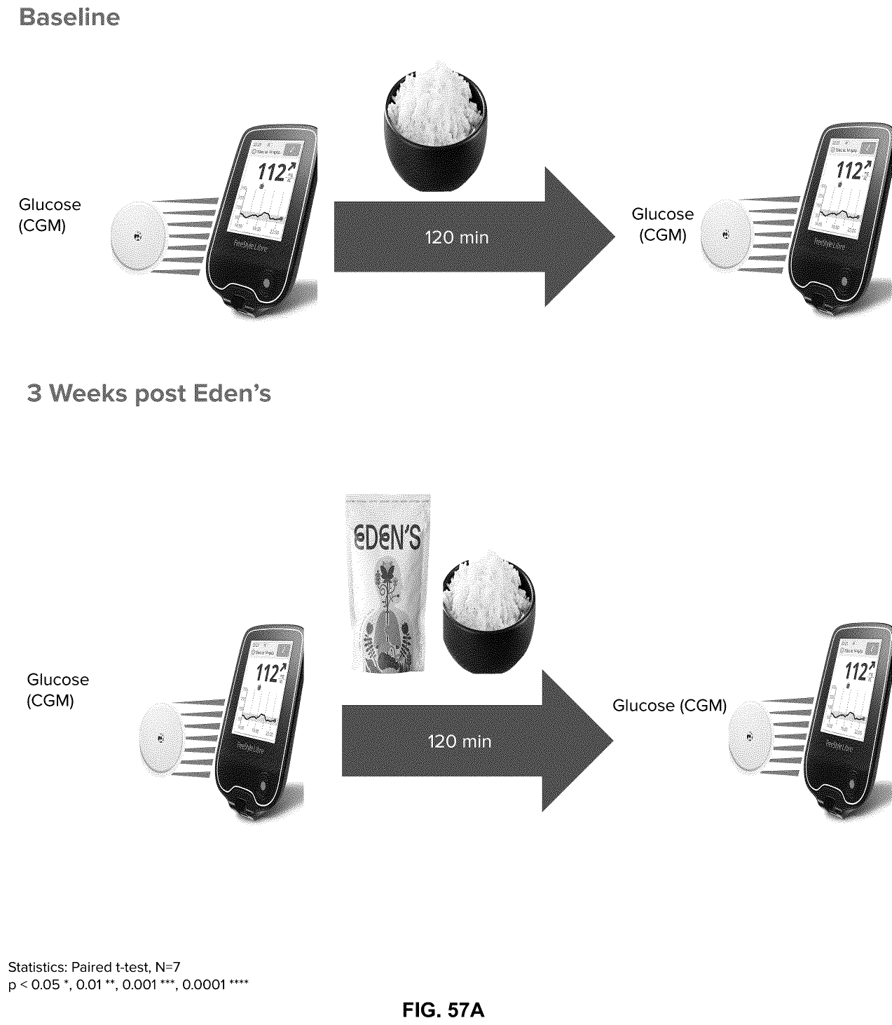

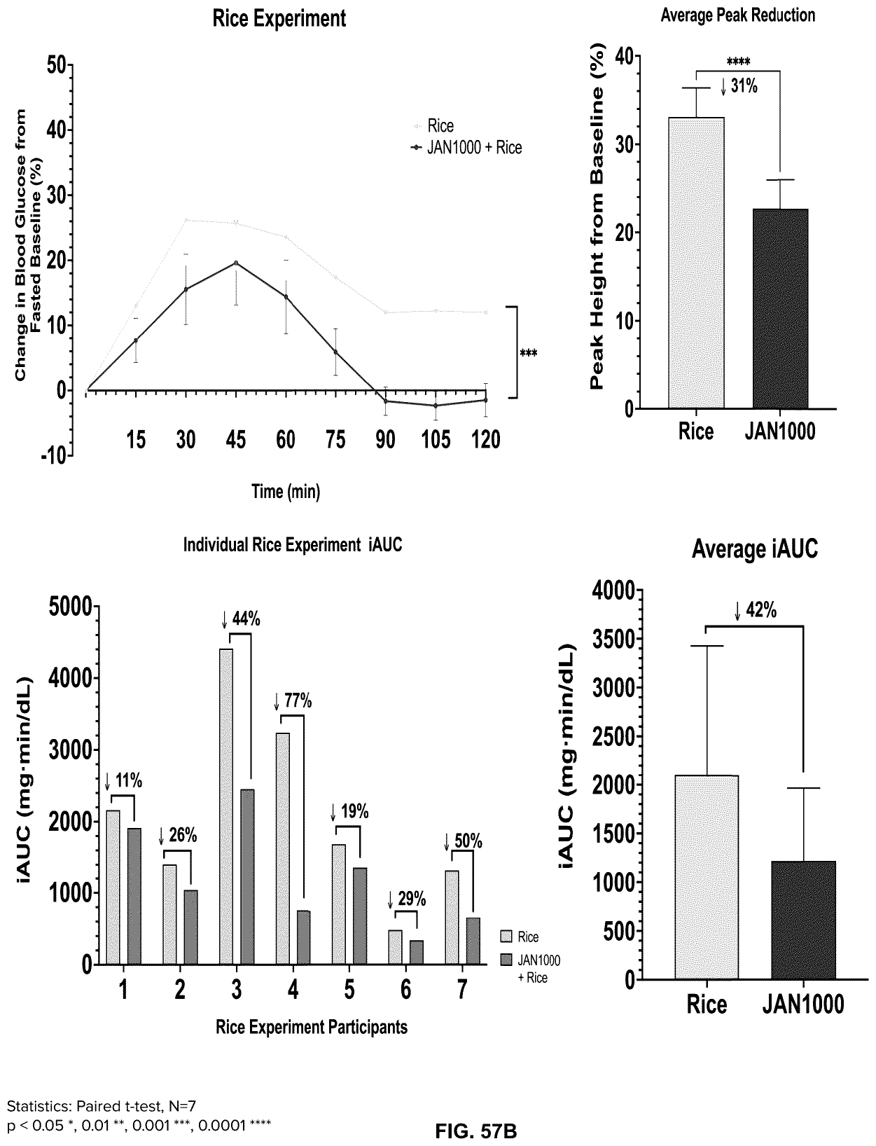

FIGS. 57A-57B illustrate a rice challenge experiment. In a control, glucose is monitored for a subject just after and 120 minutes after eating rice. The same test is performed to a group three weeks after using the JAN1000 supplement. Results illustrate that supplementation with JAN1000 suppresses peak glucose excursion by 10% and iAUC by 42%.

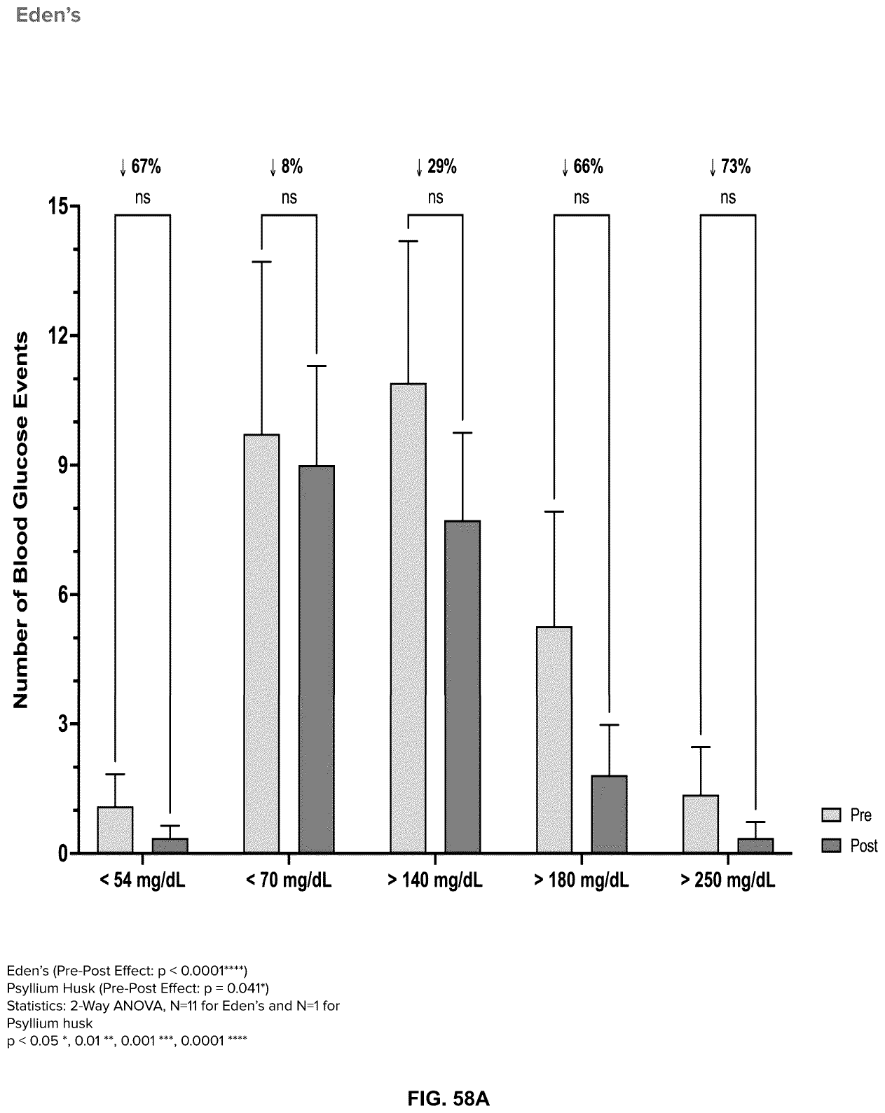

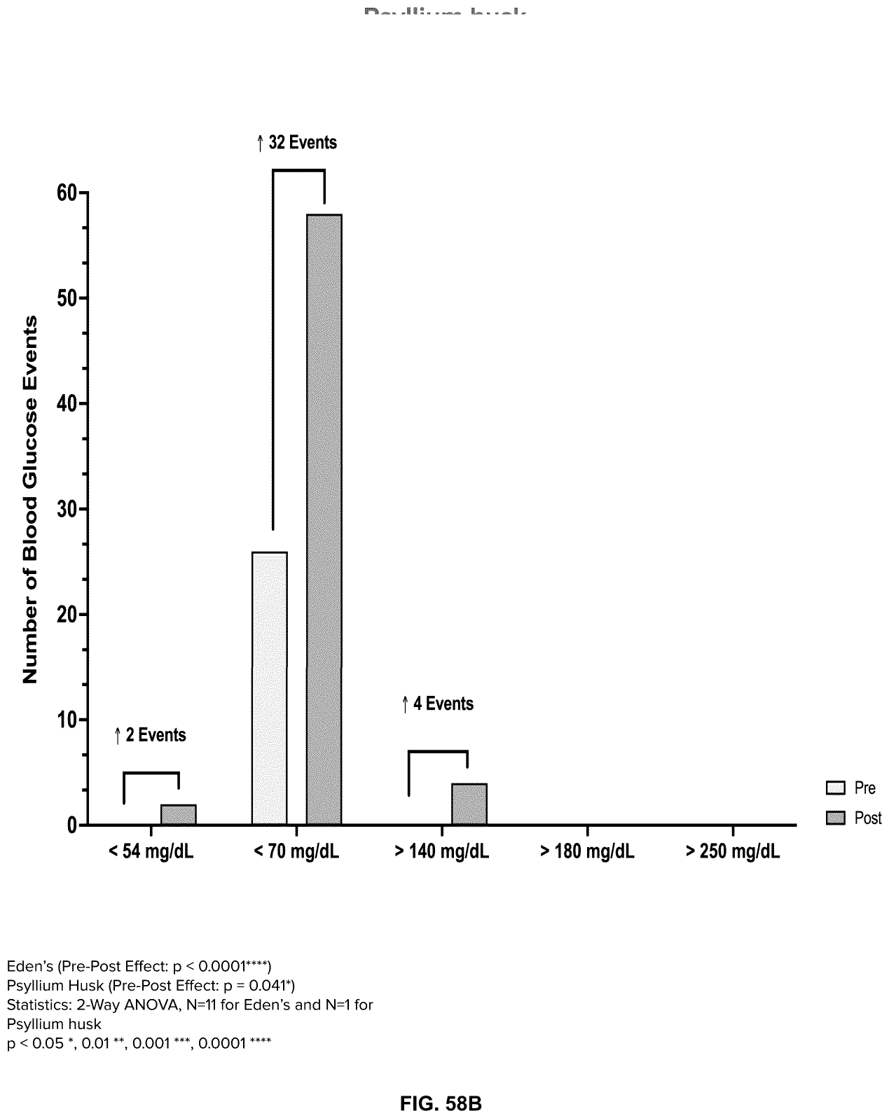

FIGS. 58A-58B illustrate comparative effects of JAN1000 and Psyllium husk on hyperglycemic and hypoglycemic episodes. The plots show that JAN1000 reduces frequency of hyperglycemic and hypoglycemic episodes.

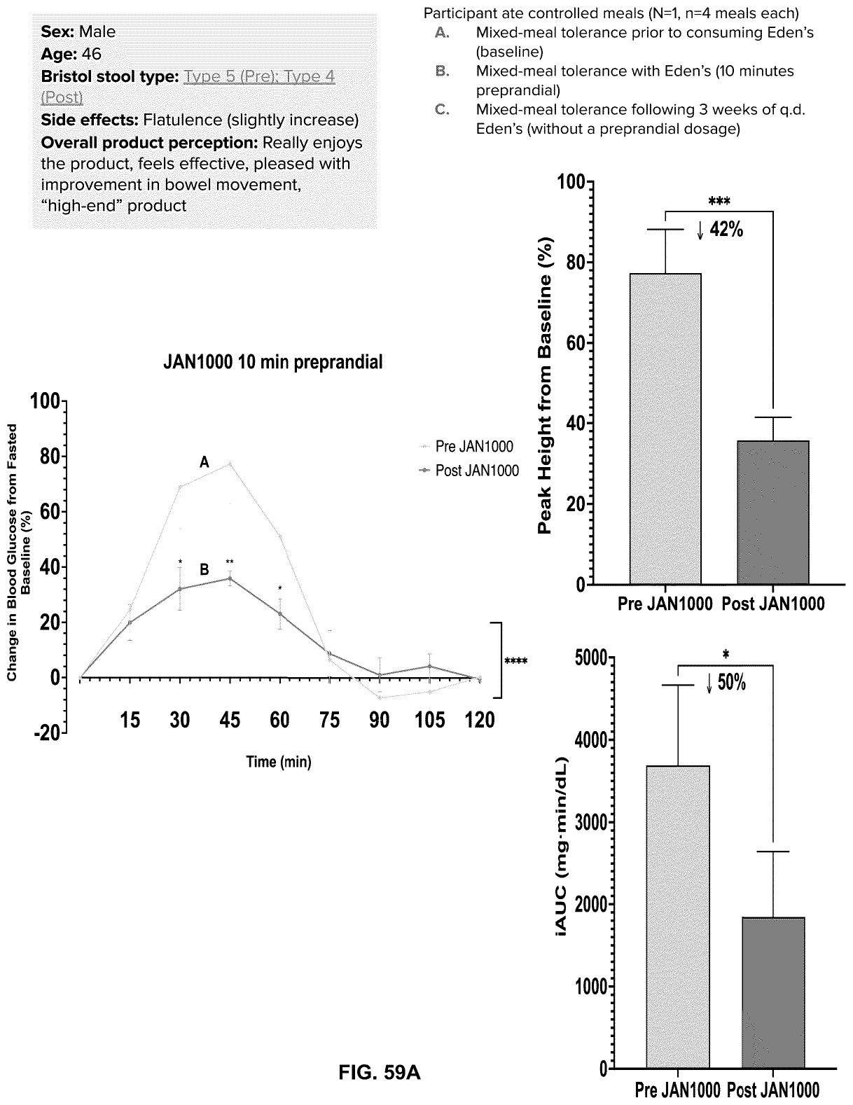

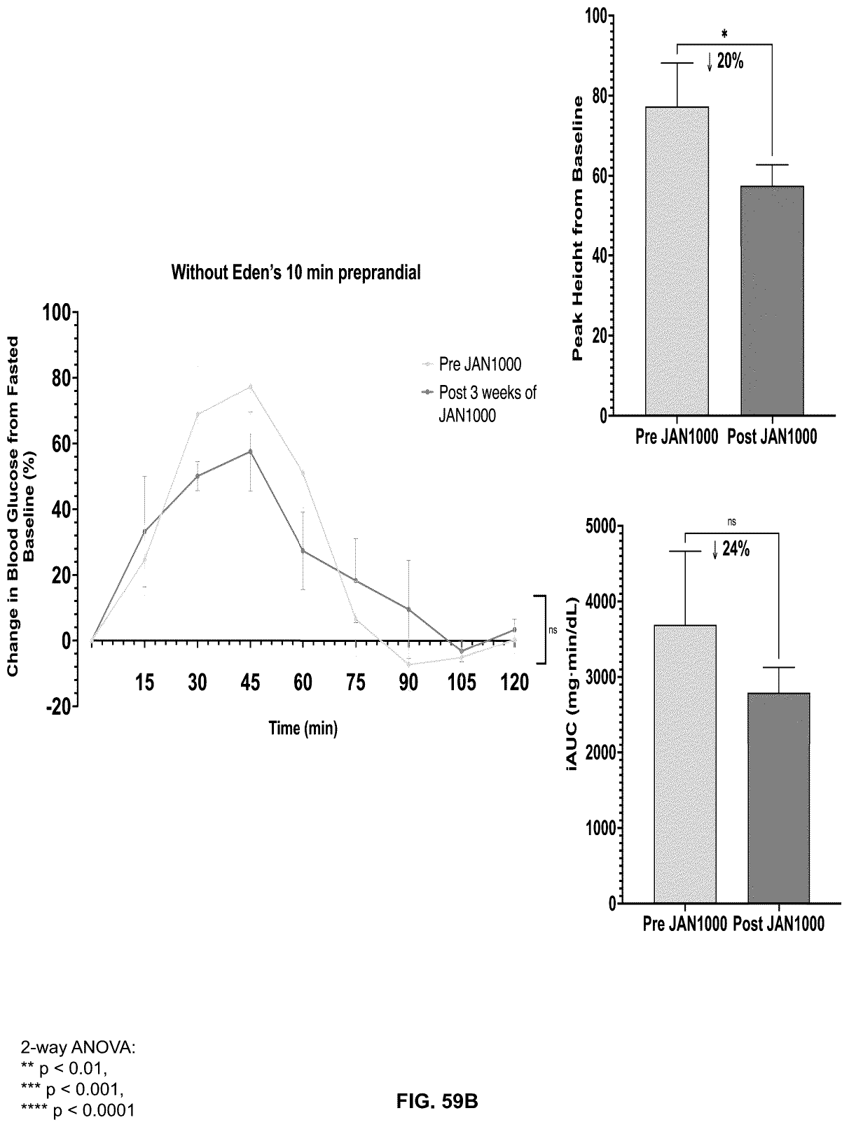

FIGS. 59A-59B illustrate a case study of JAN1000's effects on a healthy participant. The study shows that JAN1000 reduces glucose excursions acutely and after repeated use. The case study suggests that JAN1000 has the potential to improve insulin sensitivity

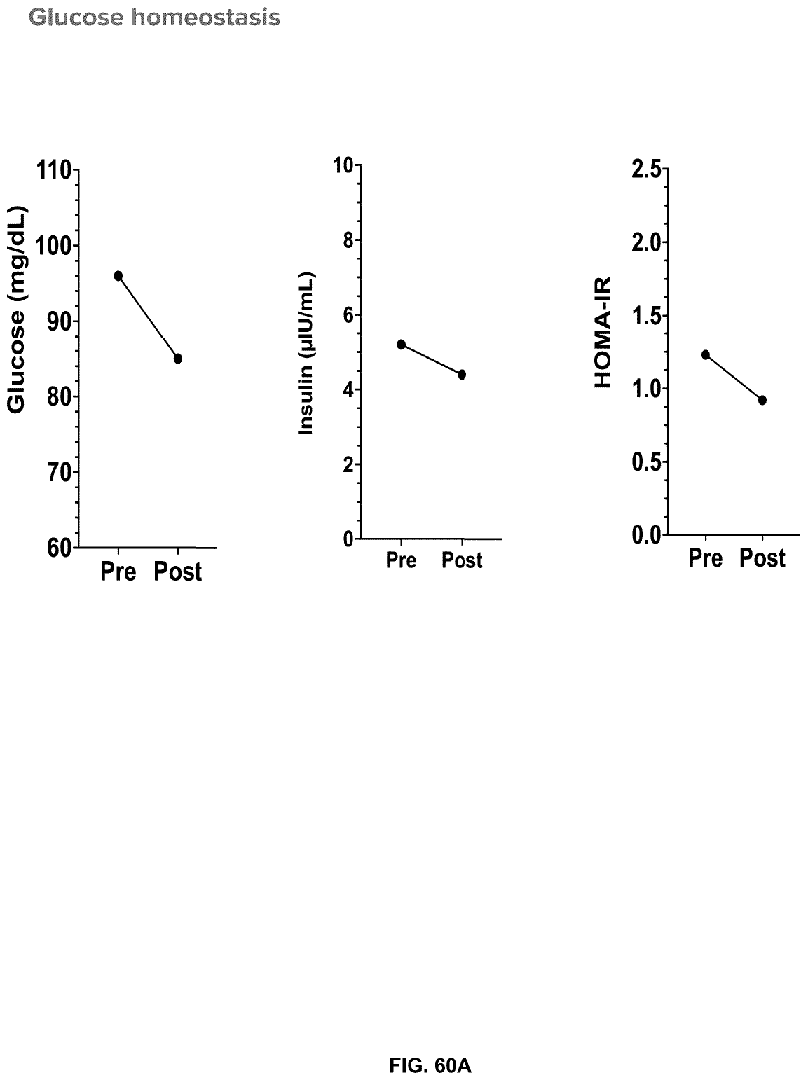

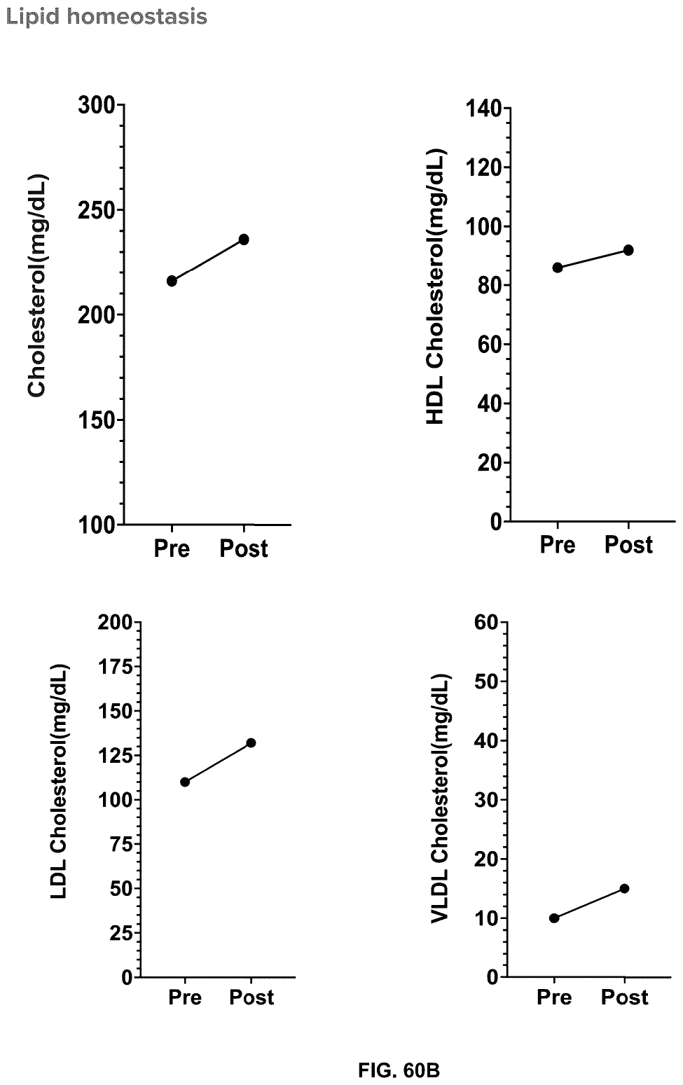

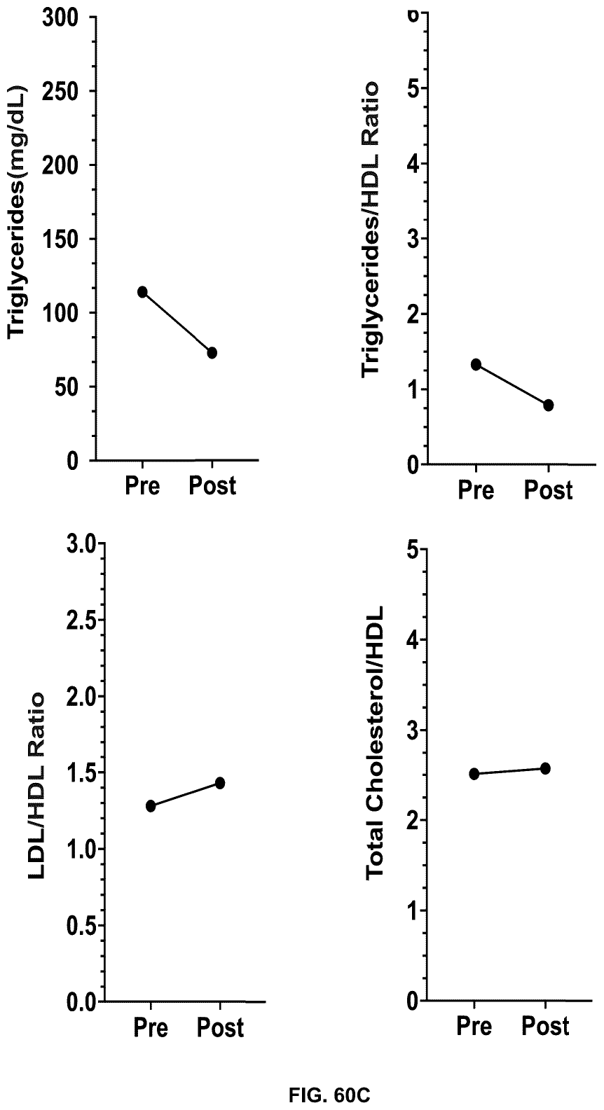

FIGS. 60A-60C illustrate additional implications of the case study from FIGS. 59A-59B. These results show that JAN1000 improves glucose homeostasis and overall metabolic health.

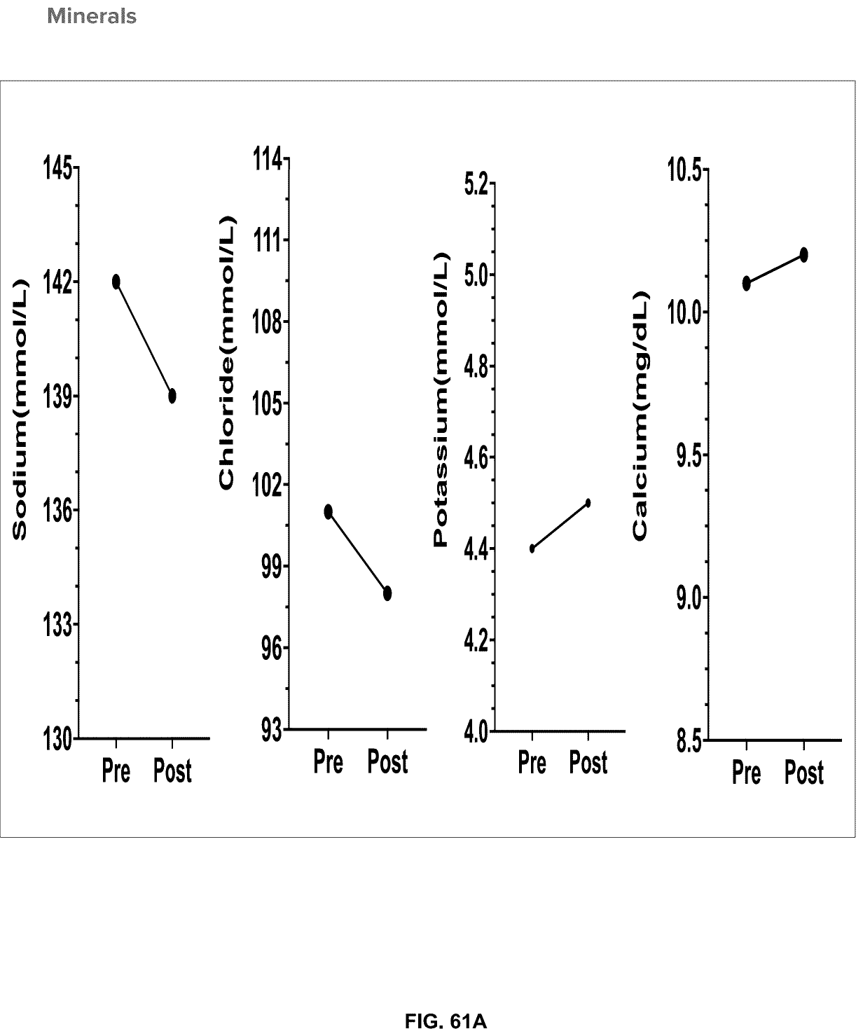

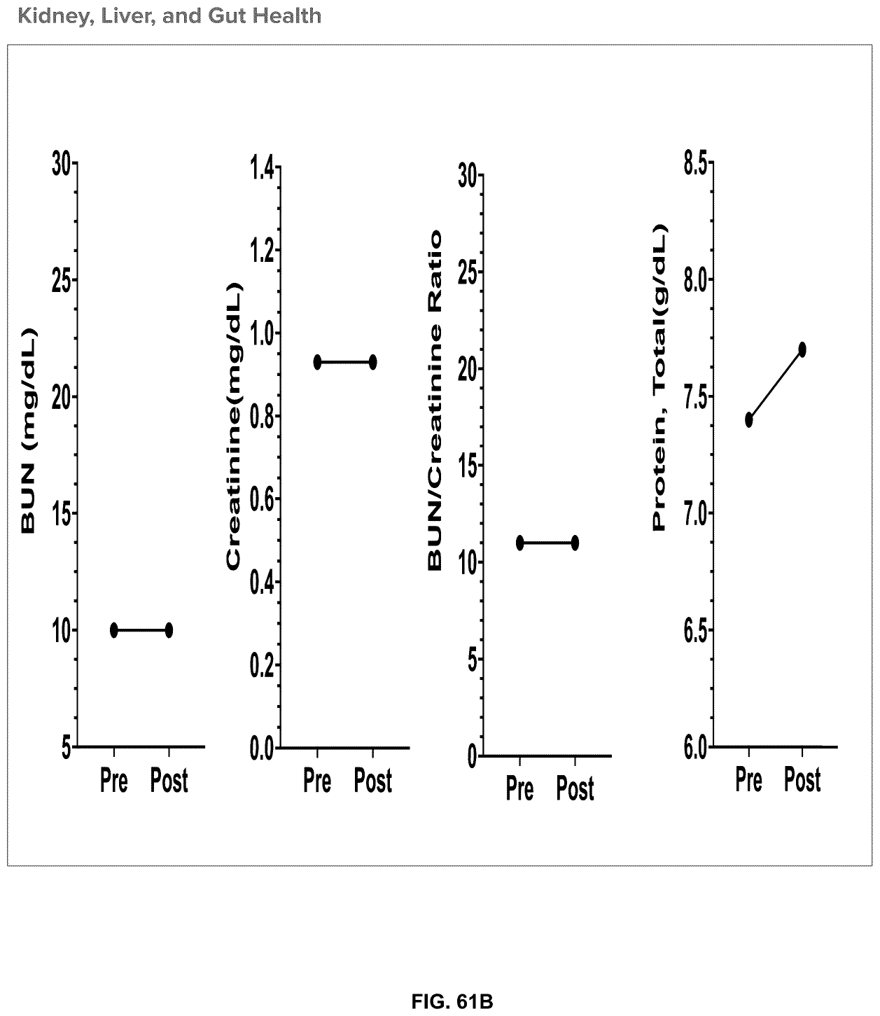

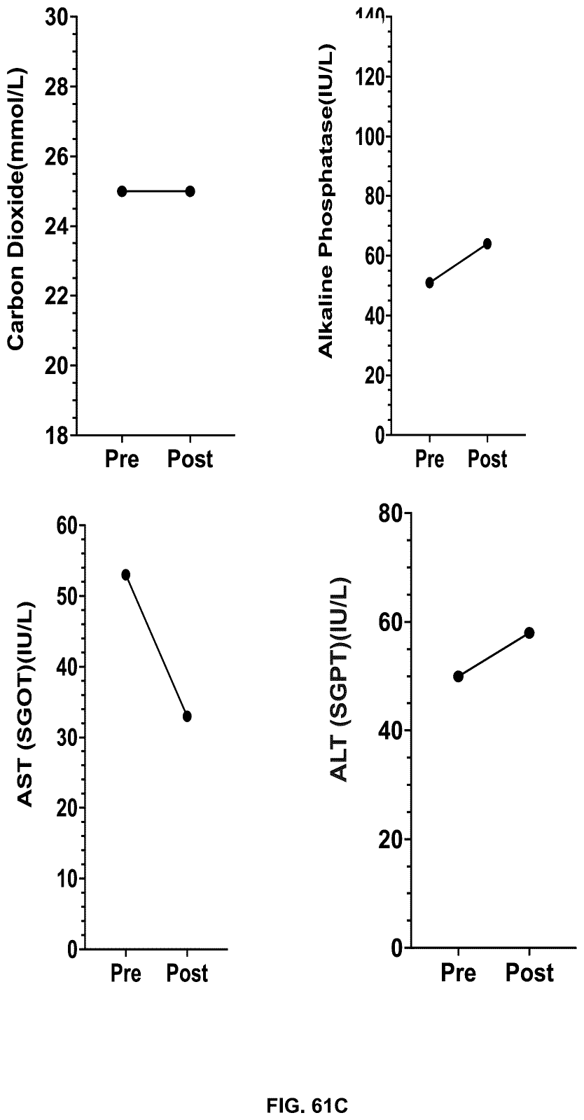

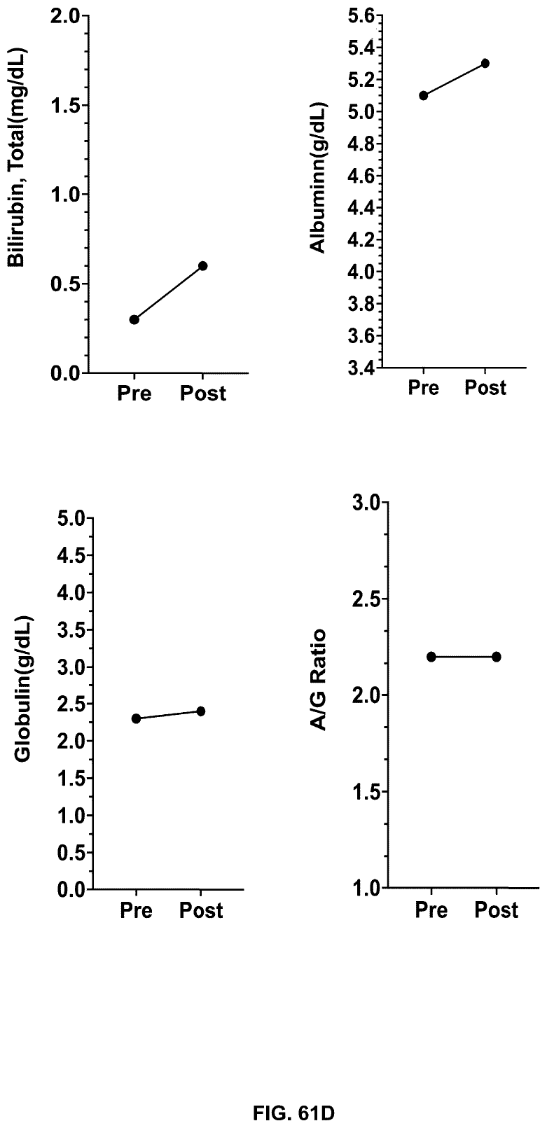

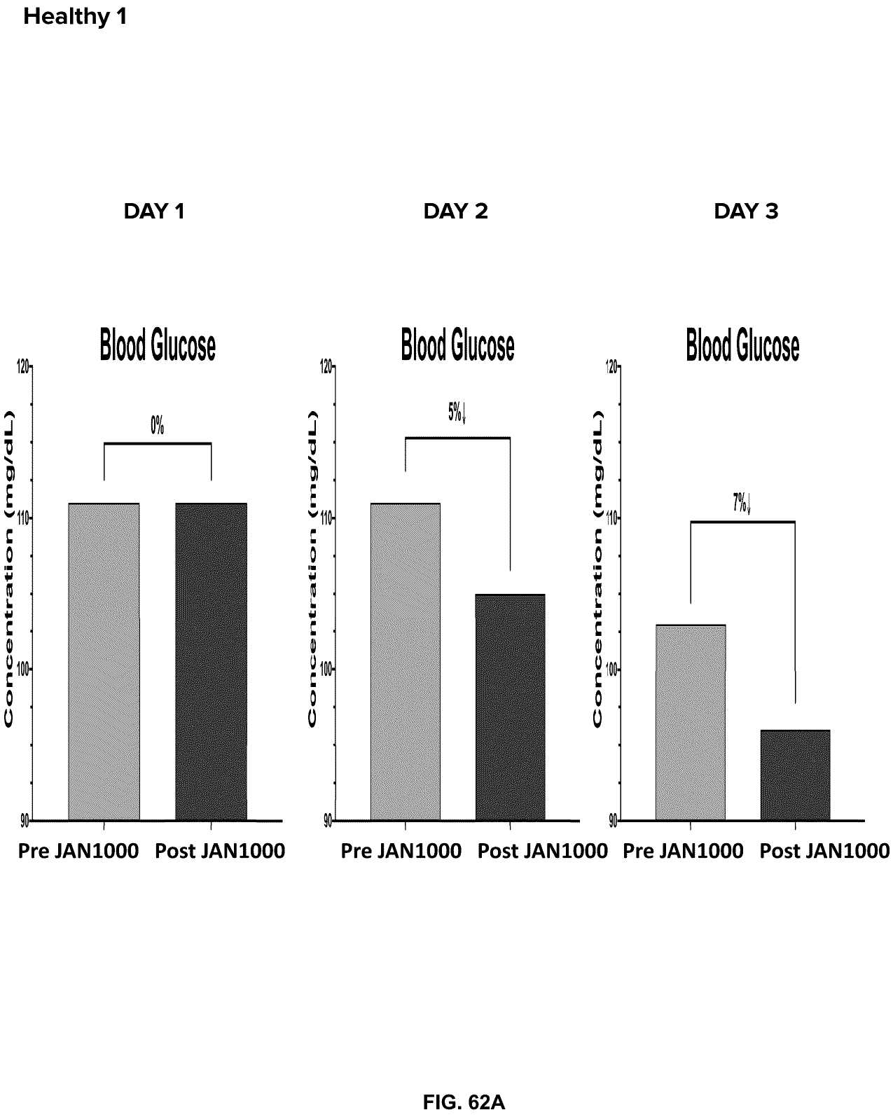

FIGS. 61A-61D illustrate additional implications of the case study from FIGS. 59A-59B. The plots show that JAN1000 modulates clinical chemistry biomarkers relevant to metabolic health.

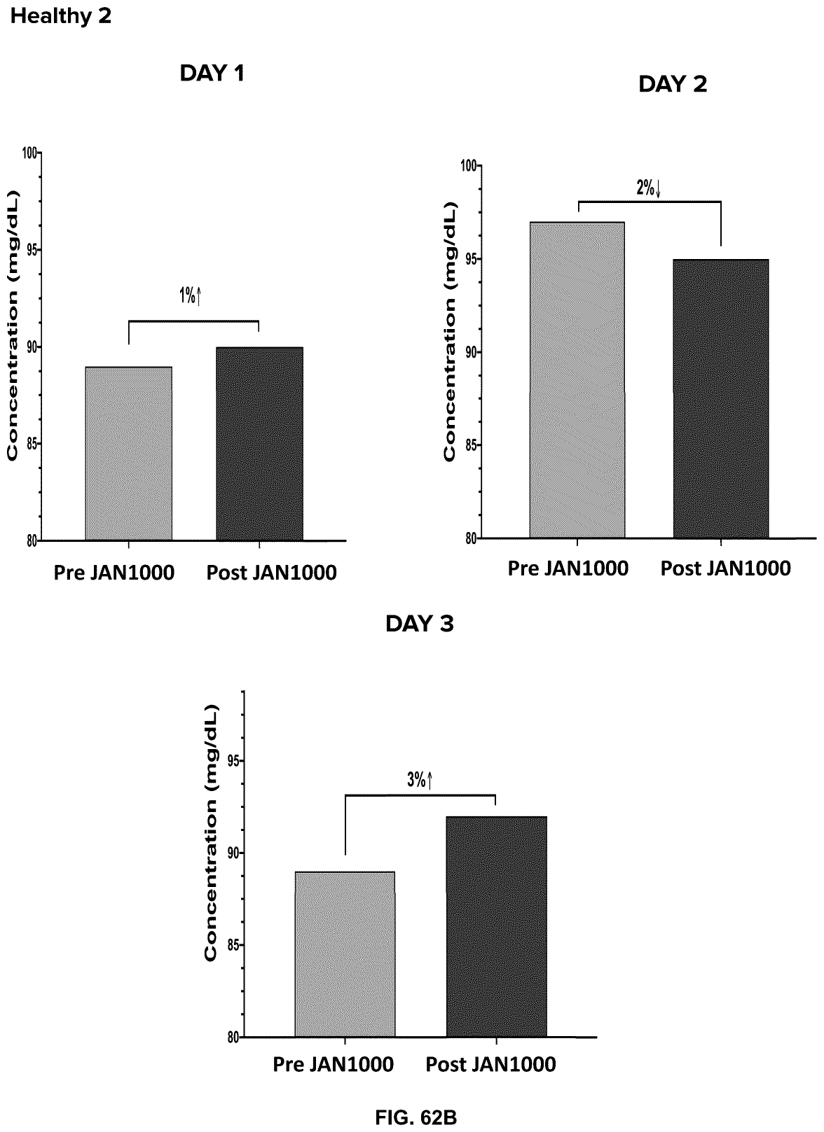

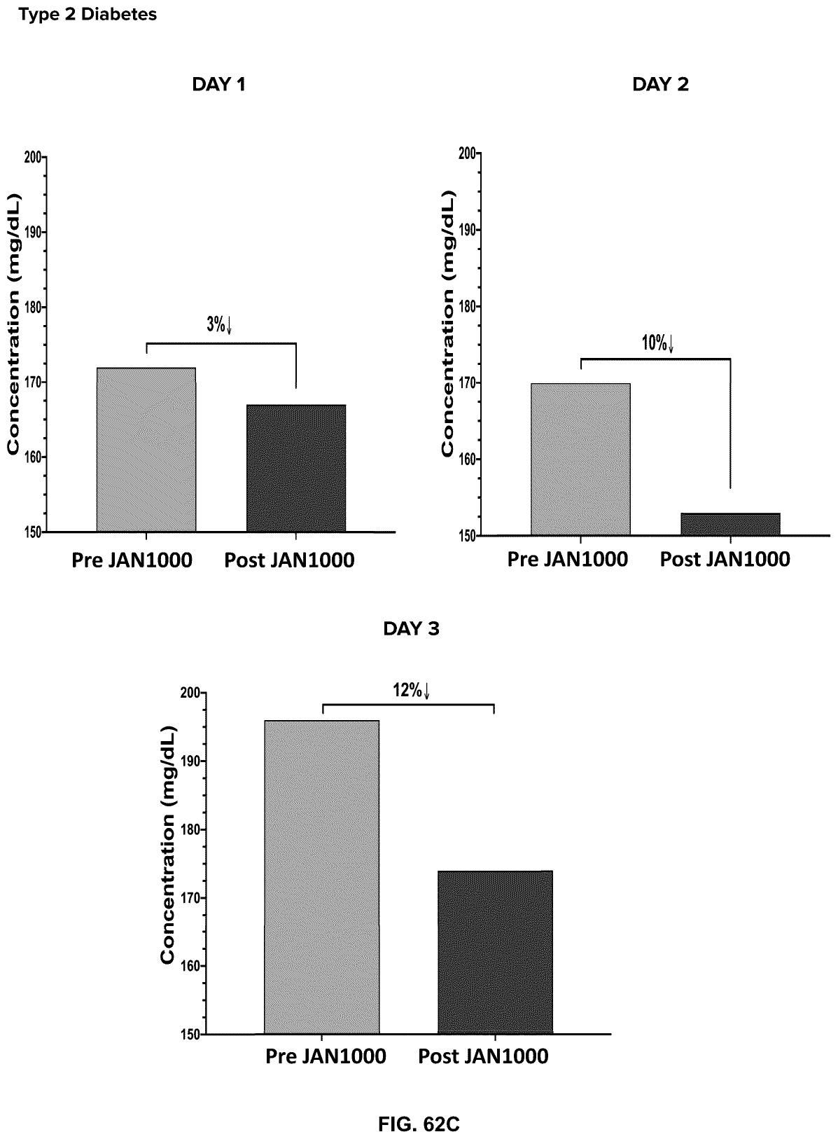

FIGS. 62A-62C illustrate an experiment to determine whether JAN1000 spikes blood glucose in healthy and participants with type 2 diabetes. During this experiment, blood samples were taken in non-fasted state, at a consistent time of day in the afternoon for 3 consecutive days. Blood glucose was taken just after taking JAN1000 and 30 minutes after via a glucometer. The experiment showed a single serving of JAN1000 trends to improve blood glucose levels. The experiment concluded that single serving of JAN1000 either keeps blood glucose stable or decreases. With continued usage, there is a greater percent of reduction in healthy participant 1 and in the participant with type 2 diabetes.

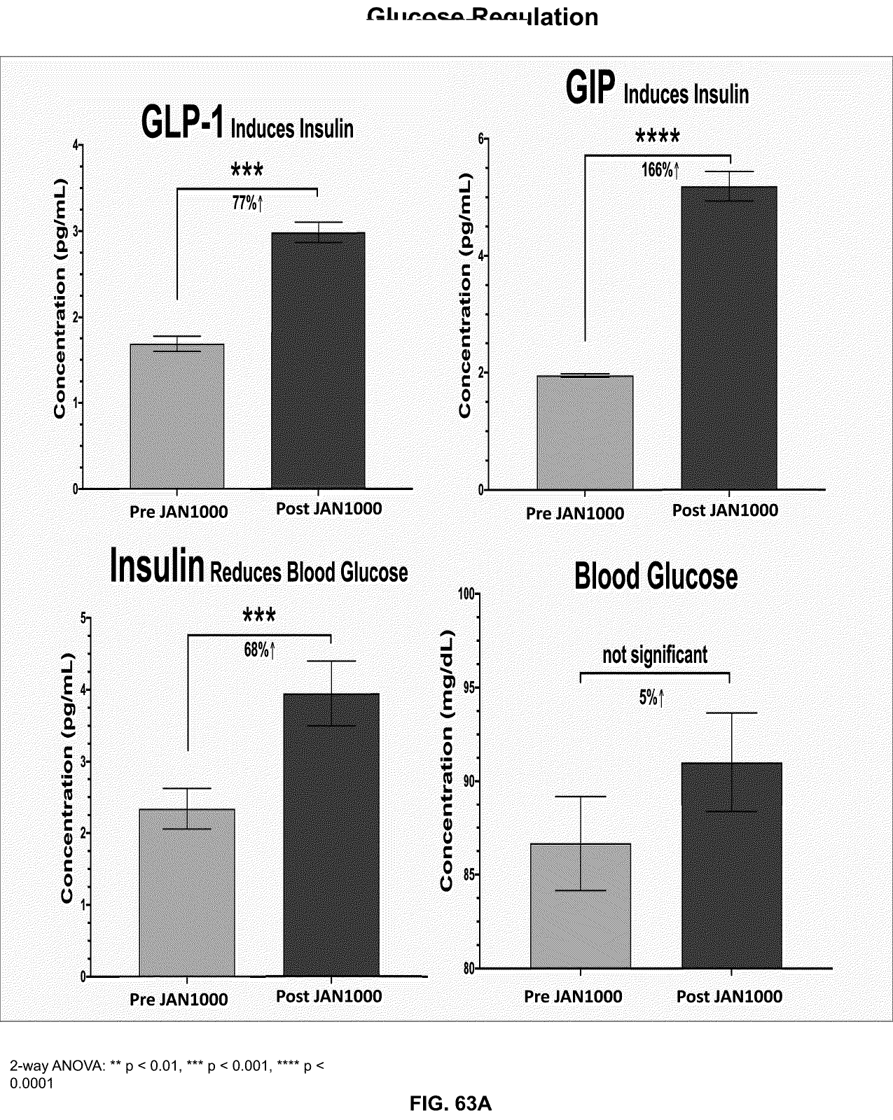

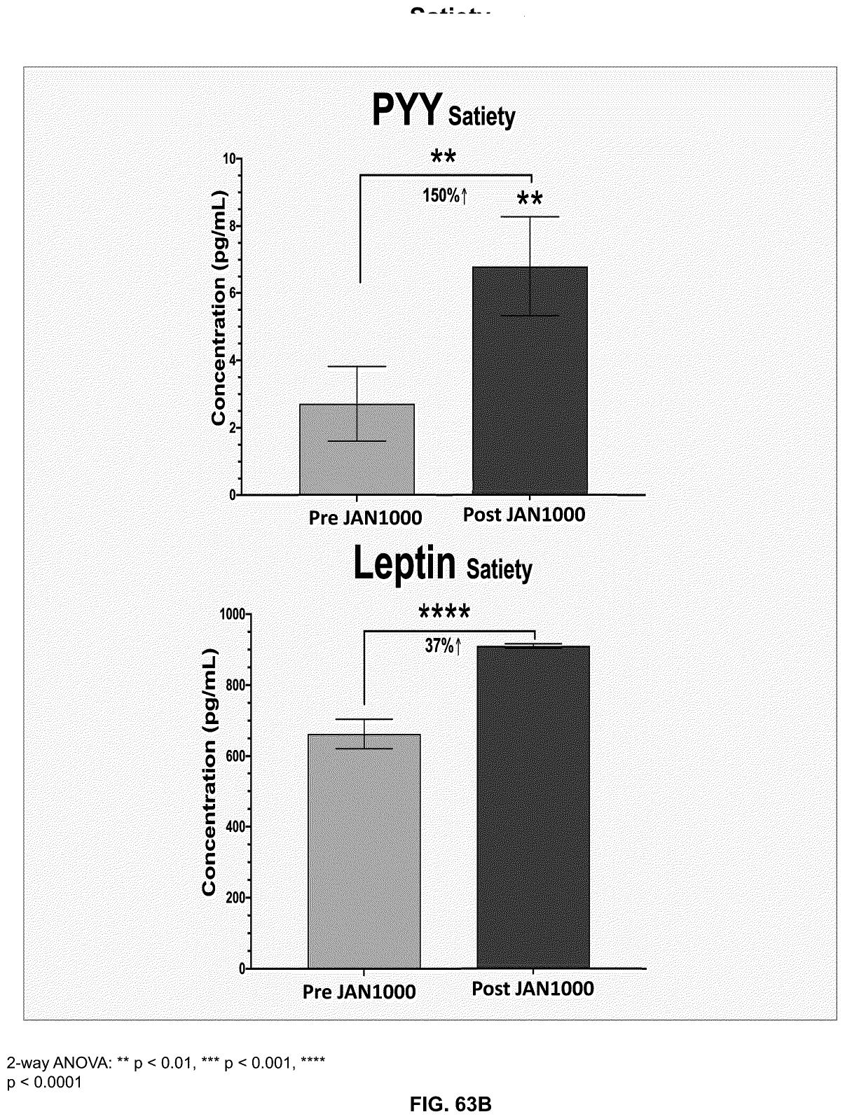

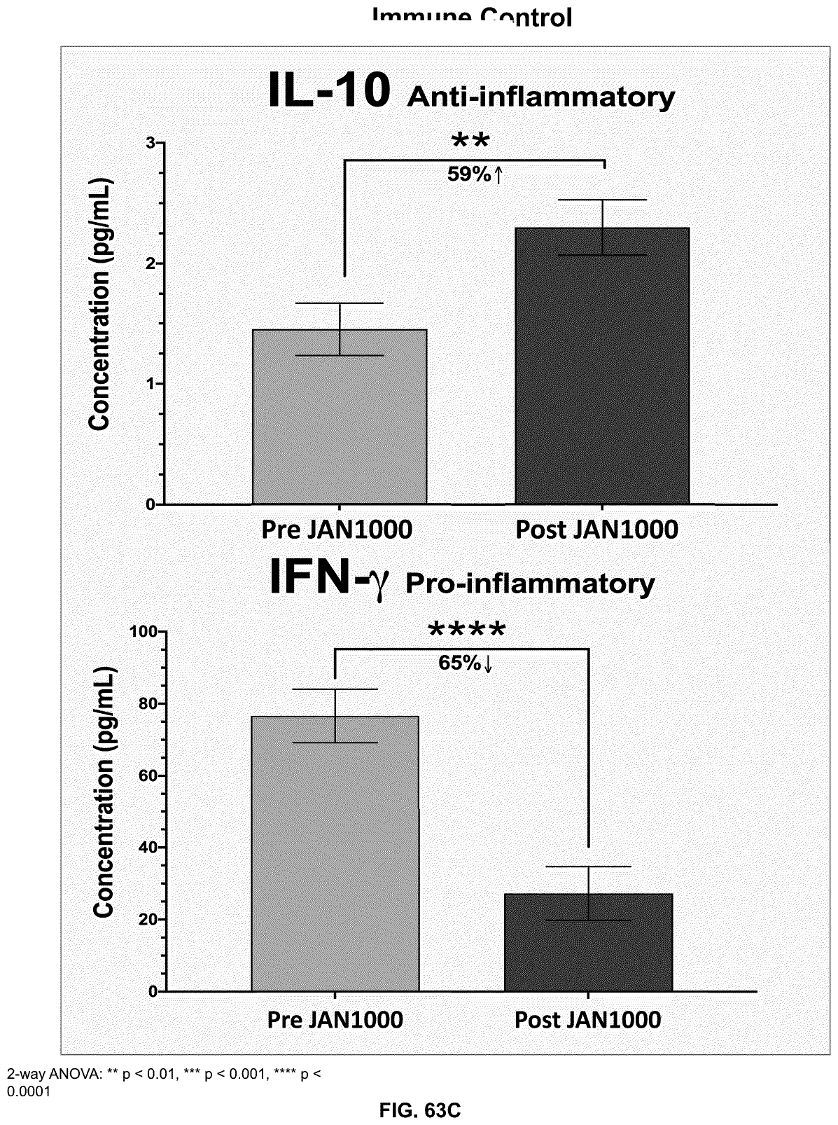

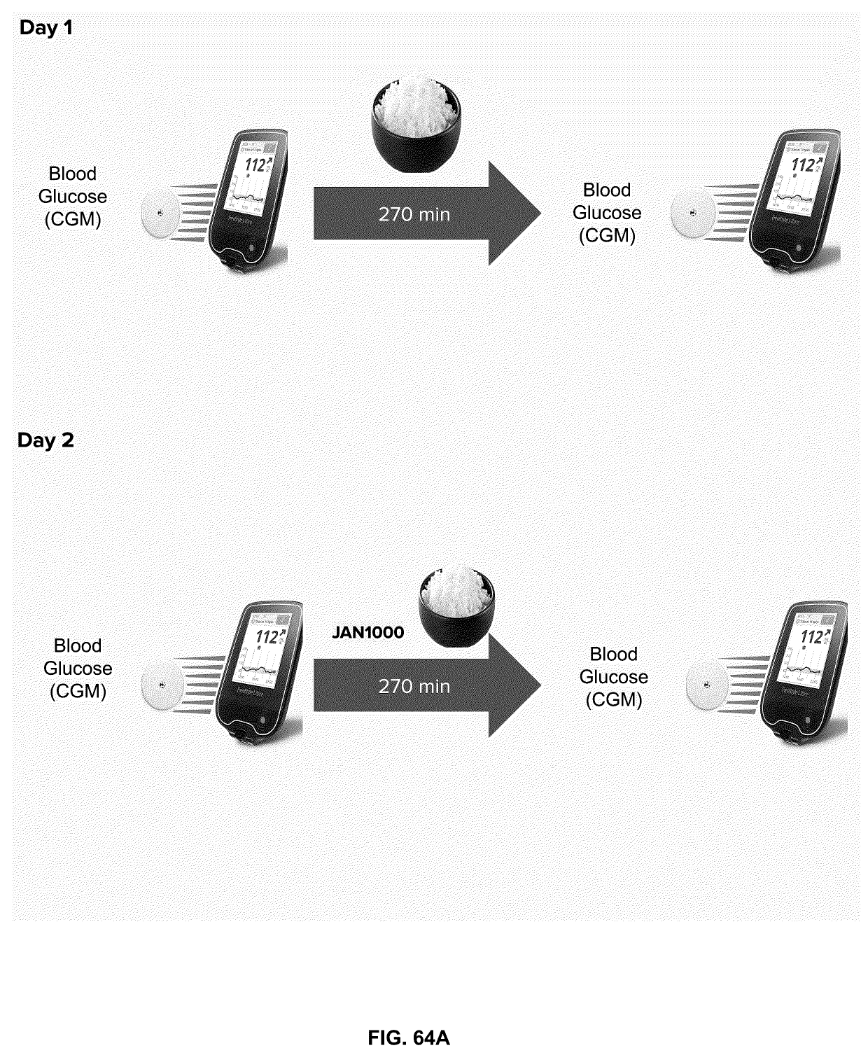

FIGS. 63A-63C illustrate an experiment to determine if a single serving of JAN1000 modulates key hormones, peptides and cytokines involved in glucoregulation, satiety, and immune control in a healthy participant. During the experiment, blood samples were taken in the morning, following an overnight fast and in a sedentary position.

Blood glucose was taken just after taking the JAN1000 supplement and after 45 min via glucometer in triplicates. For the sample, 1 milliliter (mL) of whole blood was drawn with protease and dipeptidyl peptidase-4 (DPP-IV) specific inhibitors. The results show a single serving of JAN1000 modulates key biomarkers in a healthy participant.

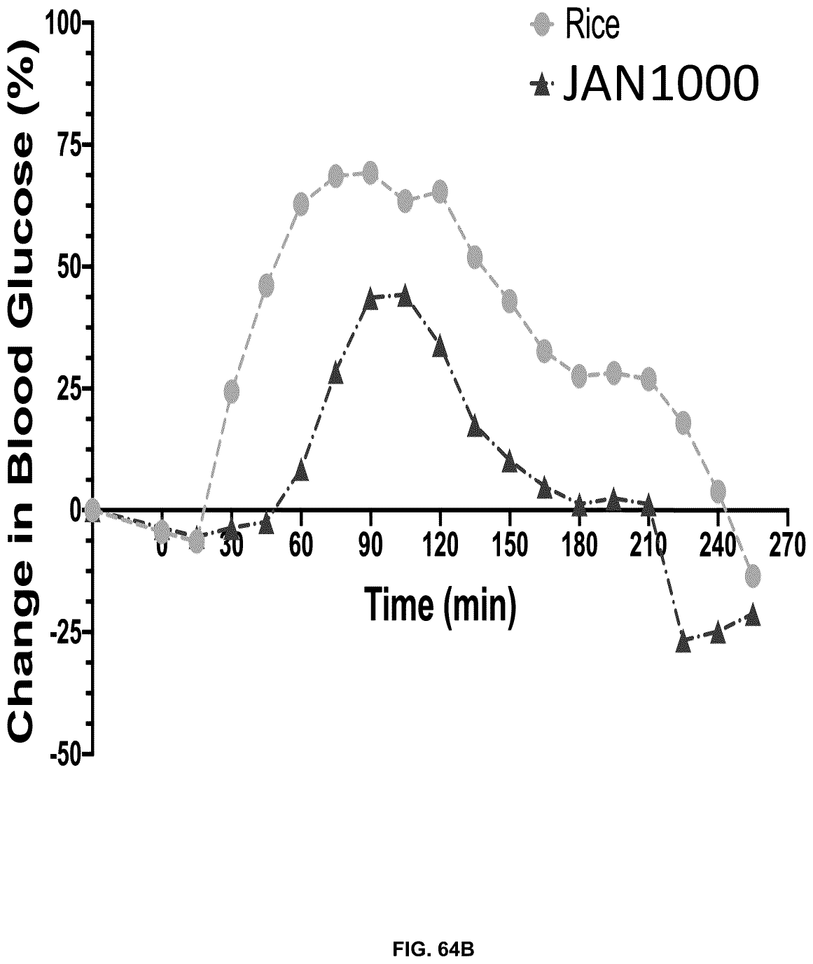

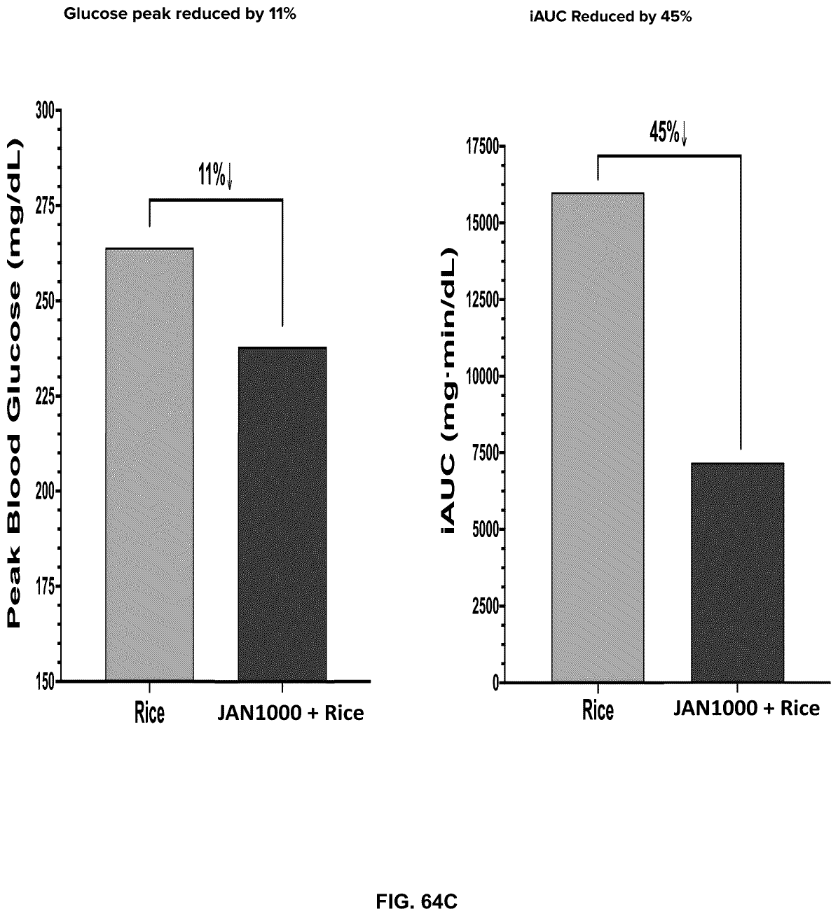

FIGS. 64A-64C illustrate another iteration of the rice experiment. On a first day, blood glucose measurements were taken just after and 270 minutes after eating a serving of rice. On a second day, a glucose measurement was taken just after ingestion of JAN1000 and the bowl of rice, and again after 270 minutes. The experiment showed that single serving of JAN1000 suppresses glucose excursion by 45% in a participant with type 2 diabetes.

In summary, JAN1000 significantly improves fasted blood glucose, time-in-range, and insulin sensitivity. JAN1000 also promotes improvement in lipid homeostasis, by reducing VLDL and LDL cholesterol and inducing HDL. JAN1000 reduces glucose excursions induced by a standardized meal of white rice across healthy individuals. JAN1000 reduces hypervariability in blood glucose excursions by reducing hypoglycemic and hyperglycemic events, especially compared to Psyllium husk which exacerbates glucose variability. JAN1000 provides a holistic metabolic health outcome for a healthy and type 2 diabetes participant, by reducing glucose excursions, improving lipid homeostasis, and improving bowel function.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-88. (canceled)

89. A composition comprising:
(i) a polysaccharide selected from the group consisting of resistant potato starch, locust bean gum, oat bran, Galacto-oligosaccharides, apple fiber, orange fiber, barley bran, oat fiber, pea fiber, chia fiber, kudzu, tara gum, konjac gum, beta glucan, guar gum, partially hydrolyzed guar gum, gum Arabic, and soluble corn fiber;
(ii) a bacteria species selected from the group consisting of Lactobacillus paracasei, Lactobacillus rhamnosus, Bacillus coagulans, and Saccharomyces boulardii; and
(iii) a member selected from the group consisting of yellow kiwi fruit extract, turmeric extract, green kiwi fruit extract, lychee fruit extract, and green tea extract.

90. The composition of claim 89, further comprising resistant potato starch, locust bean gum, oat bran, beta glucan, and guar gum.

91. The composition of claim 89, further comprising Lactobacillus paracasei, Lactobacillus rhamnosus, and Bacillus coagulans.

92. The composition of claim 89, further comprising yellow kiwi fruit extract, turmeric extract, green kiwi fruit extract, lychee fruit extract, and green tea extract.

93. A method of promoting health in a subject, comprising administering to the subject an effective amount of a composition comprising:
(i) a polysaccharide selected from the group consisting of resistant potato starch, locust bean gum, oat bran, Galacto-oligosaccharides, apple fiber, orange fiber, barley bran, oat fiber, pea fiber, chia fiber, kudzu, tara gum, konjac gum, beta glucan, guar gum, partially hydrolyzed guar gum, gum Arabic, and soluble corn fiber;
(ii) a bacteria species selected from the group consisting of Lactobacillus paracasei, Lactobacillus rhamnosus, Bacillus coagulans, and Saccharomyces boulardii; and
(iii) a member selected from the group consisting of yellow kiwi fruit extract, turmeric extract, green kiwi fruit extract, lychee fruit extract, and green tea extract.

94. The method of claim 93, wherein the composition further comprises resistant potato starch, locust bean gum, oat bran, beta glucan, and guar gum.

95. The method of claim 93, wherein the composition further comprises Lactobacillus paracasei, Lactobacillus rhamnosus, and Bacillus coagulans.

96. The method of claim 93, wherein the composition further comprises yellow kiwi fruit extract, turmeric extract, green kiwi fruit extract, lychee fruit extract, and green tea extract.

97. The method of claim 93, wherein promoting the health further comprises promoting gut health, treating metabolic syndrome, glucose regulation, promoting satiety, promoting immune function, or lipid control.

98. The method of claim 93, wherein promoting the health further comprises increasing production of a short-chain fatty acid in a digestive tract of the subject.

99. The method of claim 93, wherein promoting the health comprises increasing an abundance of a keystone microbial species in a digestive tract of the subject.

100. The method of claim 99, wherein the keystone microbial species is selected from the group consisting of Faecalibacterium prausnitzii, Akkermansia muciniphila, Collinsella aerofaciens, Eubacterium hallii, Bacteroides thetaiotaomicron, Roseburia hominis, and Eubacterium rectale.

101. The method of claim 93, wherein promoting the health comprises decreasing an abundance of a pathogenic microbial species in a digestive tract of the subject.

102. The method of claim 101, wherein the pathogenic microbial species is selected from the group consisting of Clostridioides difficile, Shigella sonnei, Escherichia coli, Campylobacter, Shigella flexneri, Shigella dysenteriae, Shigella boydii, Campylobacter gracilis, Citrobacter freundii, and Citrobacter braakii.

103. The method of claim 93, wherein promoting the health further comprises treating antibiotic-induced dysbiosis.

104. The method of claim 93, wherein promoting the health further comprises increasing a ratio of Bacteroidetes to Firmicutes in a digestive tract of the subject.

105. The method of claim 93, wherein promoting the health further comprises increasing a secretion of GLP-1, PYY, or IL-10 in a digestive tract of the subject.

106. The method of claim 93, wherein promoting the health further comprises increasing an expression of Gcg, Pcsk1, Pyy, Tlr4, Muc1, or Muc2 in a digestive tract of the subject.

107. The method of claim 93, wherein promoting the health further comprises improving a regularity or a consistency of bowel movements.

108. The method of claim 93, wherein the subject has diabetes or pre-diabetes.

109. The method of claim 99, wherein the subject has pre-diabetes.

110. The method of claim 99, wherein the subject has an inflammatory bowel disease or an inflammatory bowel syndrome.
